(19) United States
(12) Patent Application Publication
(10) Pub. No.: US 2026/0310656 A1
(43) Pub. Date: Oct. 8, 2026

(54) SYSTEMS AND METHODS FOR DATABASE MANAGEMENT WITH CONSTRAINED ARTIFICIAL INTELLIGENCE

(71) Applicant: Concordare Trials Inc., New York, NY (US)

(72) Inventor: Zacharia Sawaged, New York, NY (US)

(73) Assignee: Concordare Trials Inc., New York, NY (US)

(21) Appl. No.: 19/638,661

(22) Filed: Apr. 3, 2026

Related U.S. Application Data
(60) Provisional application No. 63/783,750, filed on Apr. 4, 2025.
(60) Provisional application No. 63/812,705, filed on May 27, 2025.

(51) Int. Cl. G06F 16/23

(52) U.S. Cl. CPC G06F 16/2386; G06F 16/2365

(57) ABSTRACT

Provided are systems and methods for building, managing, and executing structured data objects that provide universal compatibility for downstream systems, and in further example, maintain and propagate updates, changes, and modifications throughout associated data elements in the data structure. The changes preserve interoperability with downstream systems, preserve data integrity, and render complex unstructured data into structured data having executable objects that reduce system complexity, reduce error in execution, reduce storage requirements, and deliver improved accuracy, among other options. For example, organizing the structured data objects around procedure-level data improves data integrity and enables encoding for compatibility with multitudes of downstream non-homogeneous systems. Further embodiments separate and control data operation to leverage AI functionality under constraints that reduce hallucinations of known approaches.

### RELATED APPLICATION

This application claims the benefit under 35 U.S.C. § 119 (e) of U.S. Provisional Application Ser. No. 63/783,750, filed Apr. 4, 2025, and entitled “SYSTEMS AND METHODS FOR DATA MANAGEMENT WITH INTEGRATED ARTIFICIAL INTELLIGENCE”, and to U.S. Provisional Application Ser. No. 63/812,705, filed May 27, 2025, and entitled “SYSTEMS AND METHODS FOR DATA MANAGEMENT WITH INTEGRATED ARTIFICIAL INTELLIGENCE”, each of which is hereby incorporated herein by reference in its entirety.

### BACKGROUND

Various systems and locations support modern clinical trial practice. Conventionally, trial protocols or the guidelines and procedures for managing the execution of the trial are exhaustive documents. They often span 200 to 400 pages and represent one of the most inefficient and error-prone implementations associated with modern medicine.

### SUMMARY

The inventor has realized that there is a need for a consistent specification of all the data included in such clinical trial protocols. Such a format can be used across all medical procedures and management, and has demonstrated efficiencies in the context of clinical trial execution and integration between interdependent and often unrelated or uncooperative systems. According to one embodiment, the formatting and generation of a consistent data structure from conventional unstructured data sources representing clinical trial protocols yields computational efficiency across the various systems and integration points that support the clinical trial. In some examples, hundreds of pages of protocol specification can be compressed and reduced to a cross-referenced data structure, which limits the needed data to a fraction of the data in the unbounded and unstructured protocol. Moreover, the formatting of the data structure and cross-reference architecture makes downstream operations that rely on the encoded data vastly more efficient than conventional implementation (e.g., in terms of memory, processing, accuracy, precision, and/or consistency, among other options).

In various embodiments, the detailed data architecture and, for example, procedure-focused data units enable capture and organization of clinical trial protocols that virtually eliminate human error in subsequent operation, improve integration with various downstream systems, and further improve precision in data set generation from such downstream systems. Structured form generation is one example of operations enabled by the new architecture and format, where structured forms can be automatically created at any system in the architecture, limiting error and/or improving data completeness, among other options. In other examples, the system is configured to process clinical trial documents in any form to build user interfaces that will control template or form creation.

Further embodiments include artificial intelligence models that are trained to ingest clinical protocols in document and other formats and transform that information into the compressed structured data format. Various models can be configured to build aspects of the compressed structured data format, and the resulting transformation used to enable improved operation. The processing of the AI can also include user interface creation based on resulting .cptm files, automatic webform generation, etc. The functionality can be implemented with different models and/or consolidated models, as well as holistically trained models, among other options.

According to one aspect, the data architecture employs a node-based format in which clinical procedure objects serve as primary organizing entities. Each procedure node simultaneously carries schedule linkage to one or more visit definitions with protocol-defined timing windows, vocabulary identifiers from a plurality of independent clinical terminology standards co-resident on the same object, references to applicable protocol rule objects encoding safety, monitoring, and compliance logic, a cross-reference index to the originating protocol document section, and a deterministic anchoring point from which a plurality of downstream operational artifacts are generated. Modification of a procedure node propagates to all derived artifacts referencing that procedure. The node-based format enables a single procedure definition to link to its timing, data requirements, terminology codes, and governing rules through a structured association graph, eliminating redundancy inherent in domain-based systems where the same contextual information would otherwise be repeated across multiple domain records.

According to another aspect, AI-based creation is provided through machine learning models trained on clinical protocol data to identify procedure-based information and associated clinical trial data. An AI-assisted extraction module receives an unstructured clinical trial protocol document and produces a structured protocol data object by applying natural language processing and entity extraction. The AI-based creation may include a large language model configured to process document content, extract structured data fields from the protocol, and extract conditional logic from the protocol. The AI-based creation may further include a human validation and approval gate configured to present the structured protocol data object for human review and, upon approval, transition the object to a locked state. The AI-based creation may employ a two-tier architecture in which all artificial intelligence inference occurs within an extraction module, and a deterministic generation module produces clinical and regulatory operational documents by template instantiation and rule application against the locked structured protocol data object without artificial intelligence inference components, neural network processing components, or probabilistic computation components.

According to another aspect, rule-based creation is provided through execution of a plurality of rules to identify, validate, and structure protocol information from non-homogeneous data sources including unstructured text, formatted tables, and natural language descriptions. The rule-based creation may include mapping identified procedures to associated clinical trial data. The rule-based creation may further include encoding protocol operational logic as machine-readable data structures comprising adverse event logic tree objects encoding Boolean evaluation criteria with nested operator nodes, clinical parameter references, comparators, threshold values, and child node arrays. The rule-based creation may include safety threshold objects encoding graded severity monitoring criteria with clinical parameter identifiers conforming to an external vocabulary standard, action triggers, and severity grades. The rule-based creation may include concomitant therapy rule arrays encoding medication class restrictions with allowed, prohibited, or restricted status classifications, washout period durations, and conditional exception logic. The rule-based creation may include discontinuation criteria objects encoding evaluatable condition trees with criterion type classifications, required action arrays, and follow-up plan references. The rule-based creation may include follow-up rule objects encoding post-event clinical scheduling obligations with trigger event references, visit window arrays, required service arrays, and termination condition trees. The rule-based creation may include reproductive safety requirement objects encoding sex-specific contraception mandates, pregnancy testing schedules, action-on-positive logic, and partner requirement specifications.

According to another aspect, a confidence-gated pipeline is provided for assigning clinical vocabulary identifiers. An AI normalization step receives a raw service name and outputs a canonical name, disambiguation context, and a confidence signal. A deterministic database lookup step uses the normalized canonical name to query a vocabulary database and returns a candidate code and match confidence score. A confidence gate writes the vocabulary identifier to the structured protocol data object when the confidence score meets a predefined threshold, and flags below-threshold results for human review without writing a code. The AI normalization step is architecturally prohibited from generating vocabulary codes directly.

According to another aspect, a method for automated clinical trial site feasibility assessment is provided. The method includes receiving a plurality of validated structured protocol data objects, each representing a clinical trial protocol digitized at procedure-level granularity, each associated with a specific clinical trial site. The method includes aggregating the procedure-level data from the plurality of protocol data objects associated with each site to derive a site capability profile comprising procedure type coverage, therapeutic area coverage, protocol complexity indicators derived from structured rule objects within the protocol data, and operational throughput metrics. The method includes receiving a new structured protocol data object representing a clinical trial for which site selection is desired. The method includes comparing the requirements of the new protocol data object against each candidate site's derived capability profile at the procedure-node level. The method includes producing a ranked feasibility score for each candidate site, wherein the feasibility comparison operates on structured data elements.

Still other aspects, examples, and advantages of these exemplary aspects and examples are discussed in detail below. Moreover, it is to be understood that both the foregoing information and the following detailed description are merely illustrative examples of various aspects and examples and are intended to provide an overview or framework for understanding the nature and character of the claimed aspects and examples. Any example disclosed herein may be combined with any other example in any manner consistent with at least one of the objects, aims, and needs disclosed herein, and references to “an example,” “some examples,” “an alternate example,” “various examples,” “one example,” “at least one example,” “this and other examples” or the like are not necessarily mutually exclusive and are intended to indicate that a particular feature, structure, or characteristic described in connection with the example may be included in at least one example. The appearances of such terms herein are not necessarily all referring to the same example.

### BRIEF DESCRIPTION OF THE DRAWINGS

Various aspects of at least one embodiment are discussed herein with reference to the accompanying figures, which are not intended to be drawn to scale. The figures are included to provide illustration and a further understanding of the various aspects and embodiments, and are incorporated in and constitute a part of this specification, but are not intended as a definition of the limits of the invention. Where technical features in the figures, detailed description or any claim are followed by references signs, the reference signs have been included for the sole purpose of increasing the intelligibility of the figures, detailed description, and/or claims. Accordingly, neither the reference signs nor their absence are intended to have any limiting effect on the scope of any claim elements. In the figures, each identical or nearly identical component that is illustrated in various figures is represented by a like numeral. For purposes of clarity, not every component may be labeled in every figure. In the figures:

FIG. 1 is an example block diagram of system elements and process flow, according to one embodiment;

FIG. 2 is an example block diagram of system elements, according to one embodiment;

FIG. 3 is an example block diagram of system elements and process flow, according to one embodiment;

FIG. 4 is an example block diagram of system elements and process flow, according to one embodiment;

FIG. 5 is a logical mapping of operational efficiencies, according to one embodiment; and

FIG. 6 is a block diagram of an example system, according to one embodiment;

FIG. 7 is a block diagram of an example computer system improved by implementation of the functions, operations, and/or architectures described herein;

FIG. 8 illustrates an example process flow, according to one embodiment;

FIG. 9 illustrate an example system environment, according to one embodiment;

FIG. 10 illustrate an example system environment, according to one embodiment;

FIG. 11 illustrates an example process flow, according to one embodiment;

FIG. 12 illustrates an example process flow, according to one embodiment;

FIG. 13 illustrates example elements of a generated data structure, according to one embodiment;

FIG. 14 illustrates example elements of a generated data structure, according to one embodiment; and

FIG. 15 illustrates system execution on an example data structure with use of encoded logic elements, according to one embodiment;

FIG. 16 illustrates an example process flow, according to one embodiment;

FIG. 17 illustrate generation of custom templates derived from a data structure including machine learning fields;

FIG. 18 illustrates a flowchart of a method for creating and validating a single source of truth, according to one embodiment;

FIG. 19 illustrates a flowchart of a method for converting natural language conditional statements to executable logic modules, according to one embodiment;

FIG. 20 illustrates a system diagram of an internal architecture of a universal smart field, according to one embodiment;

FIG. 21 illustrates a high-level system architecture diagram of a clinical trial protocol processing platform, according to one embodiment;

FIG. 22 illustrates a sequence diagram of a process for context-aware resolution of smart fields, according to one embodiment;

FIG. 23 illustrates a flowchart of a method for end-to-end generation of clinical trial documents, according to one embodiment;

FIG. 24 illustrates a mapping diagram showing transformation between natural language protocol elements and structured data fields, according to one embodiment; and

FIGS. 25-26 illustrates example data in Tables I, II, III, and IV.

### DETAILED DESCRIPTION

According to various embodiments, systems and methods are provided to improve data capture, data formatting, and improved storage efficiency in conjunction with management, planning, and execution of clinical trials protocols. For example, a new structured format can be used across all medical procedures and management (regardless of data source and format), and has demonstrated efficiencies in the context of clinical trial execution and integration between interdependent and often unrelated or uncooperative systems.

According to one embodiment, the formatting and generation of a consistent data structure from conventional unstructured data sources representing clinical trial protocols yield computational efficiency across the various systems and integration points that support the clinical trial. In some examples, hundreds of pages of protocol specification can be compress and reduced to a cross referenced data structure, that limits the needed data to a fraction of data in the unbounded and unstructured protocol. Moreover, the formatting of the data structure and cross reference architecture makes downstream operations that rely on the encoded data vastly more efficient than conventional implementation (e.g., in terms of memory, processing, accuracy, precision, and/or consistency, among other options.).

Examples of the methods, devices, and systems discussed herein are not limited in application to the details of construction and the arrangement of components set forth in the following description or illustrated in the accompanying drawings. The methods and systems are capable of implementation in other embodiments and of being practiced or of being carried out in various ways. Examples of specific implementations are provided herein for illustrative purposes only and are not intended to be limiting. In particular, acts, components, elements, and features discussed in connection with any one or more examples are not intended to be excluded from a similar role in any other examples.

Also, the phraseology and terminology used herein is for the purpose of description and should not be regarded as limiting. Any references to examples, embodiments, components, elements or acts of the systems and methods herein referred to in the singular may also embrace embodiments including a plurality, and any references in plural to any embodiment, component, element, or act herein may also embrace embodiments including only a singularity. References in the singular or plural form are not intended to limit the presently disclosed systems or methods, their components, acts, or elements. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms.

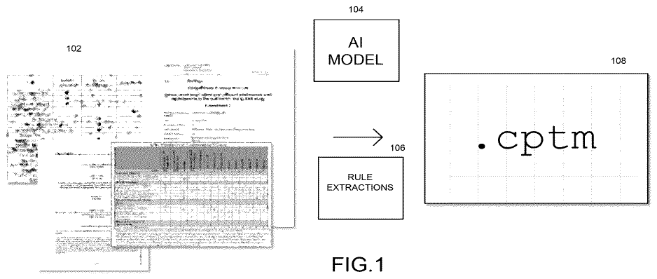

FIG. 1 shows example clinical trial protocol data 102 having a variety of source formats that can include any number of source formats, unformatted data, unstructured data, natural language data, among a host of options. The clinical trial protocol data 102 can be transformed into a structured data format 108 (e.g., “.cptm”). In various embodiments, clinical protocol data 102 can be ingested by an AI model 104 or rule based extraction processes 106. In further embodiments, the system can execute AI models and rule-based extraction processes (e.g., 104-106) on a set of ingested clinical protocol data. In various embodiments, the system is configured to accept any unstructured formatted data and identify key clinical trial protocol characteristics and map them to a structured data format (e.g., .cptm at 108).

According to other embodiments, the system can provide user interfaces to enable end-users to input clinical trial protocol data in the .cptm format. In some examples, the user interfaces are configured to guide a user through input of specific fields, define associations with procedures, and/or define associations with dates for the respective procedures to build a structured data representation in a common format.

In other embodiments, the system is configured to utilize any data encoded under the common structured data format (e.g., cptm data object). For example, the system is configured to read and write data encoded in the .cptm format. In various embodiments, ingestion of information stored in the .cptm format data structure enables the system to consistently and accurately create template checklists that span the course of an entire clinical trial protocol, and do so, automatically and with accuracies unavailable in conventional systems, from the data stored in the .cptm format.

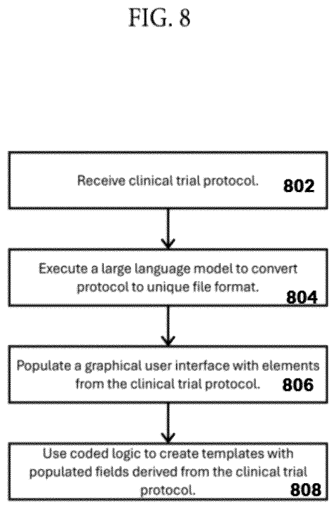

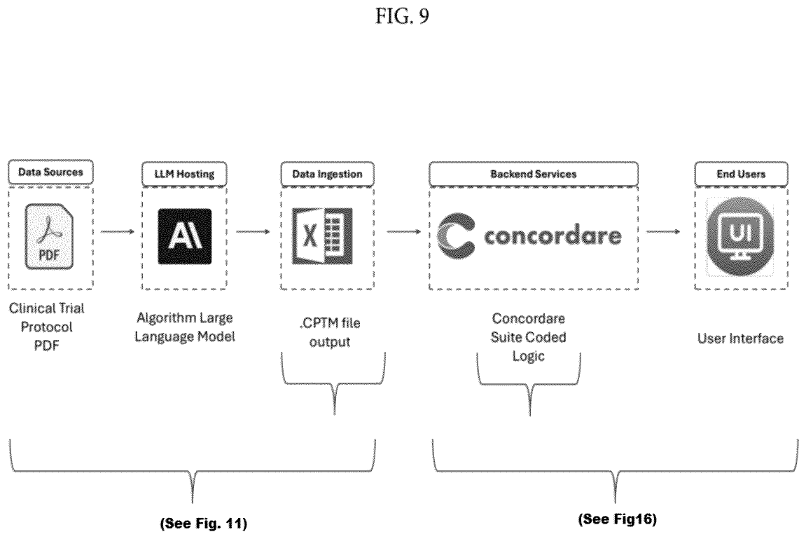

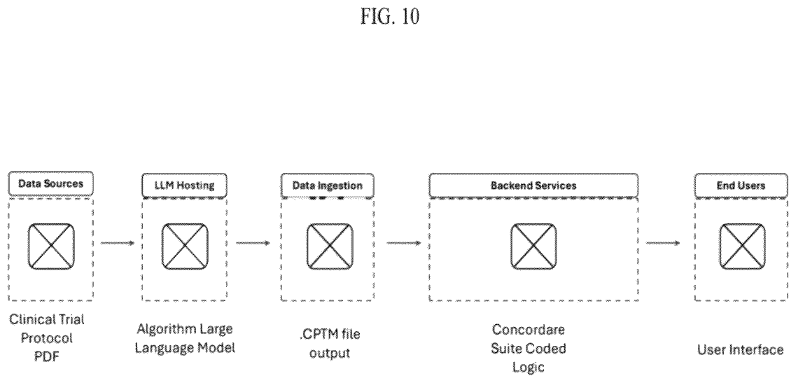

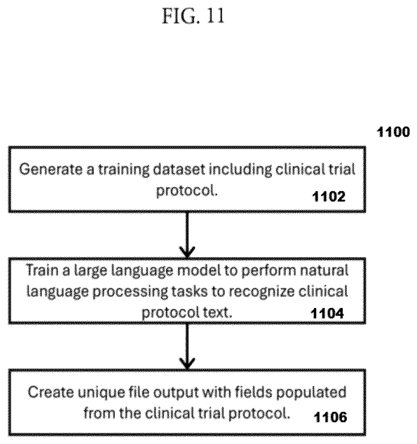

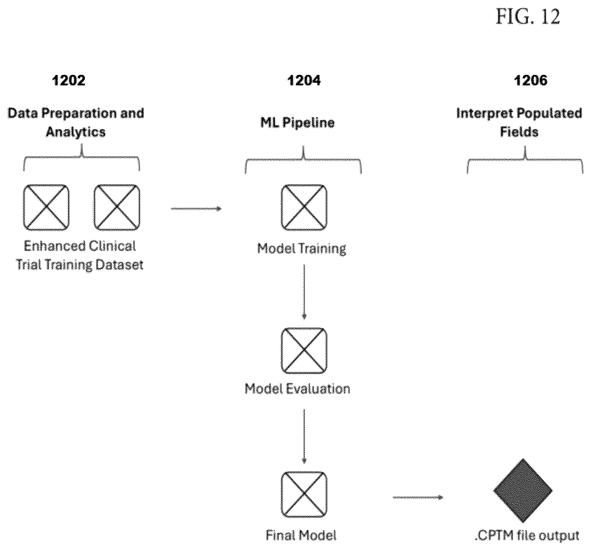

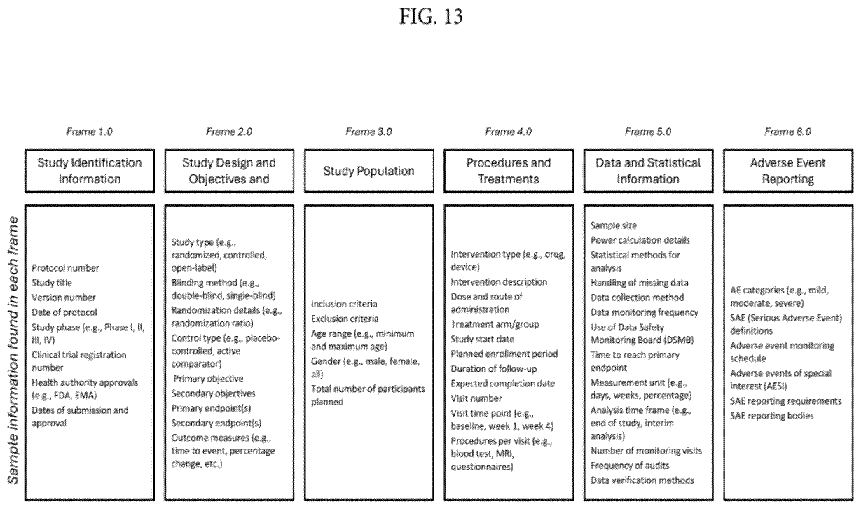

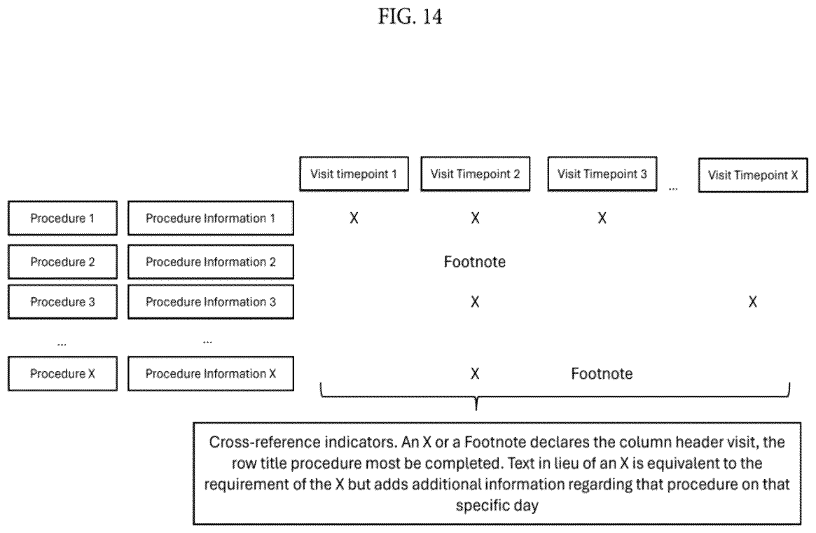

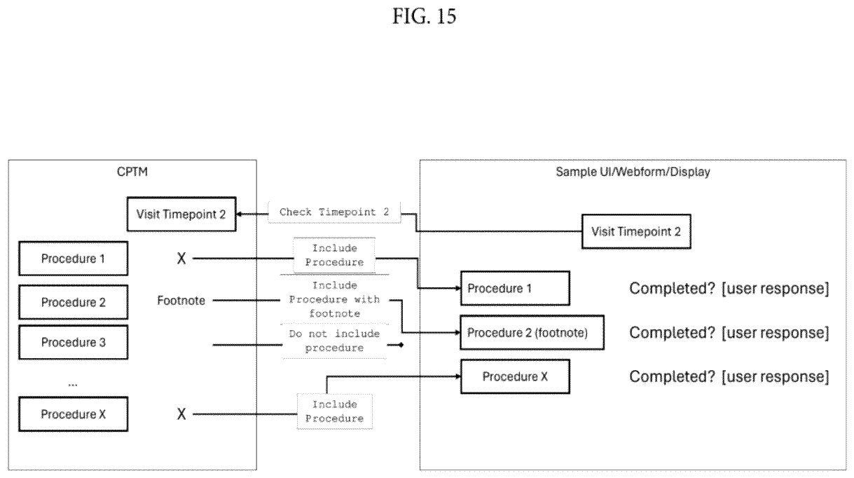

FIG. 8 illustrates another process flow that can be executed by the system. The process can begin with receipt of clinical trial data. The data can be provided to a trained machine learning model (e.g., LLM). The machine learning model can be configured to convert a variety of disparate clinical trial data sources into the .cptm format or a compressed format that maintains all source data specifically configured for downstream operations. The LLM can be trained to transform source data into a structured format that is specially configured to build graphical user interface elements that standardize subsequent operations of the clinical trial. In some embodiments, the LLM is configured to create and inject coded logic elements into the data structure that enable improved operation relative to know approaches. For example, although source documents may include a host of resulting data elements, fields, etc., the output format groups the ingested and transformed data into frames and organized the frame into parent child relationships. These structures can include coded logic at the data structure level that permit later determinations on whether the data field applies to a specific context or patient. This code logic enables the system to more efficiently build templates, GUIs, and forms on the system and/or on any connecting system, among other options. FIGS. 13 and 15 illustrate example data structures and coded logic elements created during transformation and storage of example data structures.

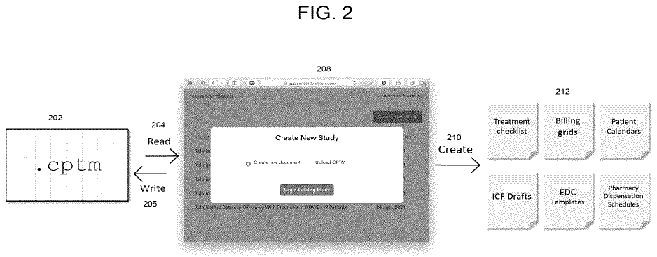

FIG. 2 shows an example process flow starting with a .cptm format data object 202 which can be read and written 204 and 205 via user interfaces presented by the system at 208. In response to user requests, the system can automatically create templates and protocol guidelines (e.g., at 210) for executing various portions of a clinical trial protocol. In one example, the system is configured to automatically build a treatment checklist based on the .cptm formatted data. In another example the system is configured to build EDC templates, ICF Drafts, pharmacy dispensation schedules, patient calendars, etc., (e.g., at 212) that span the entire course of a clinical trial protocol execution. In still other embodiments, the constructed templates can be used as execution protocols for other systems connected and used to manage the conduct of a clinical trial protocol. In further embodiments, the system is also configured to build other templates that may assist or control operation of connected systems that are used to execute a clinical trial protocol. In one example, the system is configured to build specific grids (e.g., billing grids) that reflect procedures being done and encode that information automatically for subsequent downstream systems. The systems can use these templates to control operation, manage interfaces and information presented to end users during execution.

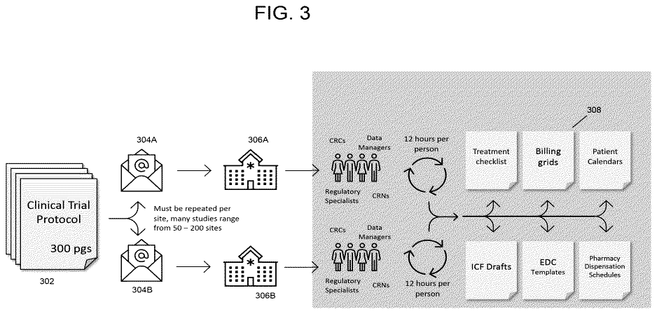

As discussed above, the system can be used to ingest unstructured clinical trial data and can also be used to build a clinical trial protocol (e.g., FIG. 2, user interfaces 208). Shown in FIG. 3 is a process flow that begins with a conventional clinical trial protocol. Conventional clinical trial protocols (e.g., 302) can span 200 to 400 pages of detailed execution information that includes redundant information, confusing instruction, even conflicting approaches that are supposed to be distributed (e.g., 304A and 304B) and executed across any number of trial sites (306A-306B). In some examples the trial sites can span 50 to 200 sites or even more. Under conventional operation, once conventional protocols have been distributed teams of personnel are tasked with turning the protocols into operational tools that can be used to execute the trial.

For example, as shown in FIG. 3, at block 308 are a plurality of CRC's, data manager, regulatory specialists, CRN's, etc. to become responsible for turning the 200 to 400 page document into operational tools. According to some examples, this processing can occupy teams of personnel at each site for multiple days representing 12 hours per person for each operational tool that is derived from a clinical trial protocol. The need to assign personnel to build operational tools (e.g., treatment checklists and resource (e.g., billing) grades, patient calendars, ICF drafts, EDC templates, pharmacy dispensation schedules, etc.) leaves conventional approaches with multiple points of failure, inconsistent and/or redundant data, and subjects the entire process flow to human error.

In various embodiments the system is configured to take the clinical trial protocol shown by way of example at 302, and transform that information into a consistent and compressed data format that is built on a procedure-based paradigm. According to some examples, by building the structured data object based on a procedure association, the system is able to generate a common format for the clinical trial protocol data that links any required procedure to other data elements. In various embodiments, this reduces the needed data for subsequent systems to a smaller footprint and smaller data representation compared to conventional approaches. In further example, this formatting compresses the required information and improves over known approaches. For example, the system can define a procedure-based object (e.g., procedure object, procedure node, etc.) in a structured data object. The procedure-based object can be encoded as a node or element in the data structure that then serves as an anchoring point for derived and/or referential data. In further embodiments, the anchoring point can refer to functionality configured to propagate changes in the procedure object to all child nodes and/or referential linked data.

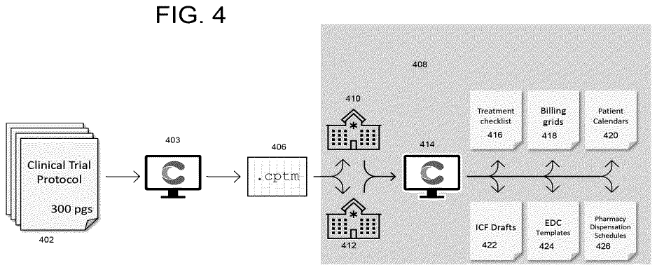

FIG. 4 illustrates an improved process flow starting with a conventional clinical trial protocol 402 that is ingested at 403 via an embodiment of the system to generate the .cptm format 404. Various system embodiments can use AI models trained on clinical protocol data to identify key features, procedure-based targets, and associate information with respective procedures to build a structured data object (e.g., .cptm) reflecting all the clinical trial protocol information in a substantially reduced size (e.g., .cptm format data object 406).

Similar to FIG. 3, clinical trial protocol data is distributed to various clinical trial sites shown in block 408. The respective clinical trial sites 410 and 412 can then automatically build operational tools because the clinical trial protocol data has been reduced to the structured data format. In some embodiments, the clinical trial sites can interact with the system 414 via application programming interfaces (e.g., API). According to some embodiments, various systems at the trial sites we each have their own APIs to connect to the system and build specific or respective operational tools including any one or more of a treatment checklist 416, grid 418, a patient calendar 420, ICF drafts 422, EDC templates for 24th, and pharmacy dispensation schedules 426, among other options.

According to various embodiments, the smaller footprint data structure improves operation of the entire network architecture by limiting the bandwidth required to communicate clinical trial protocol information, as well as distributing that same information across hundreds and sometimes even thousands of trial sites. In some embodiments, the system is configured to handle and optimize on patient data and specific data structures can be built based on the cptm format (e.g., in a .pcptm format). In further embodiments, the systems efficiency is improved computationally and with regards to networking based on the ability of local trial sites/systems to automatically construct operational tools for executing portions of a clinical trial, among other options.

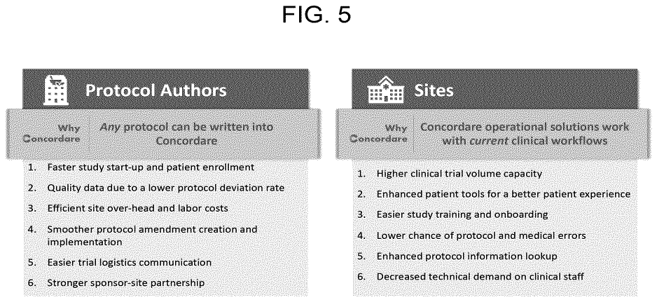

FIG. 5 illustrates example considerations in improving operation of clinical trial execution according to some embodiments of the system. From the perspective of protocol authors, the system provides interfaces to build a protocol from scratch into the common structured format. The system also provides capability to ingest already drafted clinical protocol trial data so that it is transformed into the compressed and structured format that improves operation of all downstream systems. The result is improved computation by the downstream systems, in that they operate faster and with reduced complexity, reduced computation, and reduced power in the start and management of trial execution. Such operations can improve study startup times and patient enrollment operations among other options. Further, having consistently defined data formats that are used to automatically create operational tools yields improved quality and data, improved quality data sources and reduces protocol deviation rates owing to technical complexity. Other improvements are also found in on-site efficiencies at trial locations as well as reducing operational costs both in terms of computing and labor to support execution. According to still other embodiments, the system's common format and data structure yields improvements in making adjustments or amendments during creation of a clinical trial protocol and/or during its implementation and management. For example, the common format and/or data structure elements reduce the operational burden on clinical trial management (e.g., sponsors) and the end users (e.g., respective site staff). In various embodiments, these efficiencies stem from the new elements included in the compressed format and/or .cptm format constructed via the machine learning models. Examples are illustrated in FIGS. 13 and 15.

According to another embodiment, communication between multiple systems can be reduced to a common structured data format-which enables smoother integration and reduced friction when interacting with various systems that support the execution of a clinical trial. For example, the reduced friction and communication reduces error rates throughout the system and architecture as well as reduces error rates and execution of the clinical trial. In another example, clinical trial sponsor systems can interact to leverage the common format data, execution information, among other options.

In addition, API integration enables protocol sponsor system to easily access, and readily understand and appreciate the data that is being returned or accessed. For example, integration functionality enables the sponsors to have their associated systems communicate between each other in a common format, enhancing planning and operations, and further reducing communication error or translation/data mapping issues. In further example, additional benefits can be derived from the structure enforced under the common model for a respective study sponsor. According to one embodiment, the system ensures the formatting of the data coming in from various sites is consistent and is due to the use of the system by the site or by integrating their systems using the common structured file format.

The additional efficiencies and performance improvements enable increases in clinical trial volume capacity over conventional approaches. For example, the same respective sites can execute multiple factor increases in capacity and patient volume owing to the achieved efficiencies in the system. According to some embodiments, the system enables enhanced interfaces for site staff to readily be aware of what procedures are needed for patients enrolled on a study and what data points are required from the site according to the sponsor and protocol. The common data format enables the immediate deployment and reliability of these enhanced interfaces that simplify data entry and/or analytics to support patient functionality. Other downstream effects of the improved efficiency of the system include easier study training and onboarding, lower chance of protocol errors, lower chance of medical errors during execution, enhanced protocol information lookup and analytics, and decreased technical demand on clinical staff, among other options.

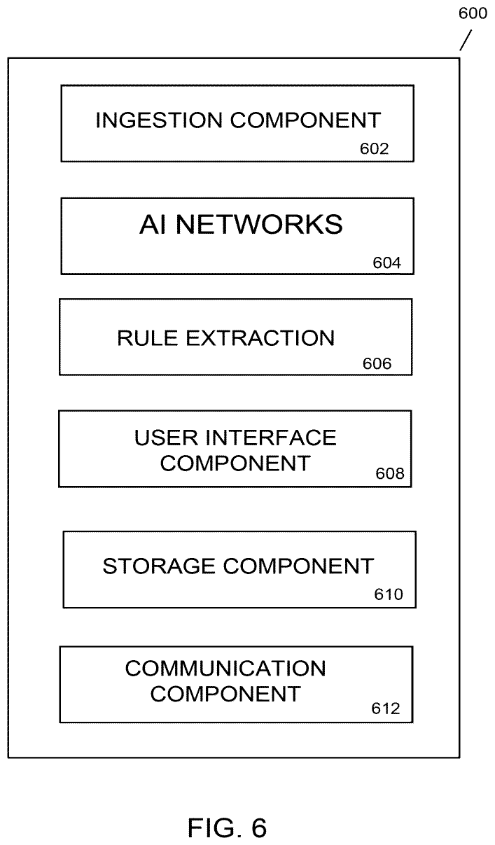

Shown in FIG. 6 is an example embodiment of the system 600. The system 600 can include a number of components that are configured to perform specific operations on the system, or the system can invoke the functionality itself without the specialized components. According to one embodiment, the system includes an ingestion component 602. The ingestion component is configured to accept as input a conventional clinical trial protocol and any associated data. In some embodiments, the ingestion component can include AI models that are configured to analyze and ingest a clinical trial protocol and any associated data to identify specific procedures, and link them to procedure-based information for storage in the compressed structured data object. The encoding of the procedure-based information can define an anchor for managing and updating all subsequent data (e.g., child nodes, nested data/objects, etc.). In still other examples, the ingestion component 602 can include rule extractors at 606 that are executed to identify specific information within a clinical protocol and map it into a compressed structured data object (e.g., .cptm). In some embodiments, the AI networks can be instantiated as a sub-component of the ingestion component. For clarity, the AI networks are illustrated as stand-alone components, but can be instantiated separately or as sub-components within embodiments of the system.

The ingestion component 602 can be configured to call and execute a variety of AI models (including for example AI networks 604). Each model may be trained to identify specific parts of the clinical trial protocol, or specific phases of a clinical trial protocol, interpret natural language, or unstructured text, found in a conventional clinical trial protocol document or any other unstructured data format for a clinical trial protocol.

According to one embodiment, a classifier type model can be used to identify specific procedures and link data associated with that respective procedure (e.g., date type of procedure, target of procedure, equipment needed for procedure, pharmacy dispensation needed for a procedure, among other options). Once trained on prior clinical trial protocols under supervised learning, various models can accept a clinical trial protocol in any unstructured format is important and output specific characteristics of the clinical trial protocol stored according to a procedure paradigm. Other unsupervised models can be used to identify sequences of operations/procedures that take place for a given patient and/or given procedure.

In other embodiments, various models can be trained according to phases of the clinical trial protocol. For example, an enrollment phase may be associated with enrollment data that specifies criteria for participants, indicators for eligibility, indicators for ineligibility, among other options. Once trained, the model can accept the clinical trial protocol and output specific information associated with an enrollment phase and procedures that may be required as part of an initial enrollment or to validate enrollment and build any clinical protocol data into the common structured data format that can be used by downstream systems.

Another model can be trained on execution phases of a clinical trial protocol and may break down the clinical protocol into various operational headings associated with execution. Other models include patient feedback models which build out a data object that includes data fields for capturing a patient's journey on the study, procedures completed, input, feedback, among other options. According to further embodiments, the system can also include rule extractors at 606 that are tailored to specific operations common to many protocol executions. The rule base extractors may look for natural language patterns or matches in unstructured data to build information into the compressed structured data object. For example, the AI system has been trained on multiple clinical trial protocols to identify the standard structure and common text headings of exclusion and inclusion criteria and provide an example processing rule that can be executed. In another example, a rule extractor can parse language in a clinical trial protocol to identify inclusion criteria for enrollment and build such material to the compressed structured data object.

According to further embodiments, system 600 can include a user interface component 608. The user interface component 608 can be configured to display user interfaces to protocol authors or other end users to facilitate construction and generation of structured data objects for use in execution of a clinical trial. According to one embodiment, the user interface component 608 can be configured to display interfaces for building a trial protocol from scratch. The user interfaces are configured to guide the user through defining only the necessary data properties for the clinical trial protocol that are required for the compressed structured data object.

According to further embodiments, the user interface component 408 can also be configured to provide user interfaces for uploading or importing unstructured clinical protocol data. In other examples, the user interfaces can be configured to identifying or submit identification of different systems in which clinical protocol trial data is located. For example, the system can include a series or a plurality of APIs that connect other systems that may manage or have data associated with the clinical protocol. In various settings, the API informs operational systems built on the requirements of the protocol, so instead of a manual coding of each, they can ingest the CPTM and be immediately programmed.

The user interfaces enable end-users to access such systems via the application programming interfaces and ingest clinical protocol data into the compressed structured data object used by subsequent systems.

The output of the various other components can be managed via a storage component 610. According to various embodiments, the storage component 610 can be configured to capture and build compressed structured data objects from information input into user interface as well as and/or ingested clinical trial protocol data.

Some embodiments the system 600 can include a communication component 612. The communication component 612 can be configured to connect the system 600 to various networks (e.g., 604), APIs, and/or downstream systems (e.g., trial site systems). According to some embodiments, the communication component 612 can be configured to distribute a compressed structured data object to a plurality of trial sites for creating operational tools that manage execution of various portions, phases, procedures, etc., of respective clinical trial protocols. In still other embodiments, the communication component 612 can be configured to manage interaction with the various systems that may be used in executing a clinical trial. The communication component can include a plurality of APIs, and each API tailored to communicate with the respective system under respective format and bridge any unstructured information to transform into the compressed structured data object. As discussed above, the system 600 can improve efficiency over known approaches by limiting the data required to be communicated over the connected architecture, limiting data communicated to respective sites as well as by reducing the data required for storage, and the capacity required for storing any respective trial protocol, among other options.

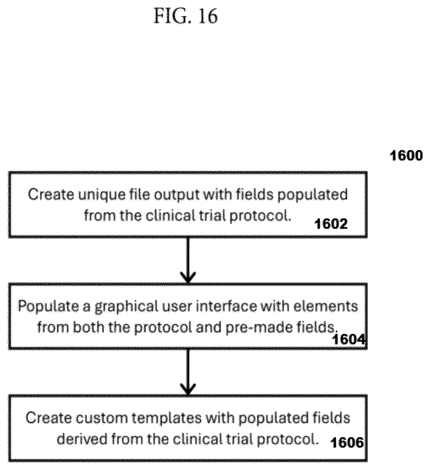

FIG. 16 illustrates a process flow for generating unique file outputs and GUI that can be based on a respective clinical trial protocol, respective site for a clinical trial protocol, respective patient participating in a clinical trial protocol. The capability of the file format, unique fields, and coded logic enables the system to generate custom templates on a site by site basis, operator by operator basis, as well as on a patient by patient basis. Interfaces can be tailored dynamically to each context (e.g., patient, trial protocol, trial site, trial personal, operator, etc.) yielding technical implementation available on conventional systems.

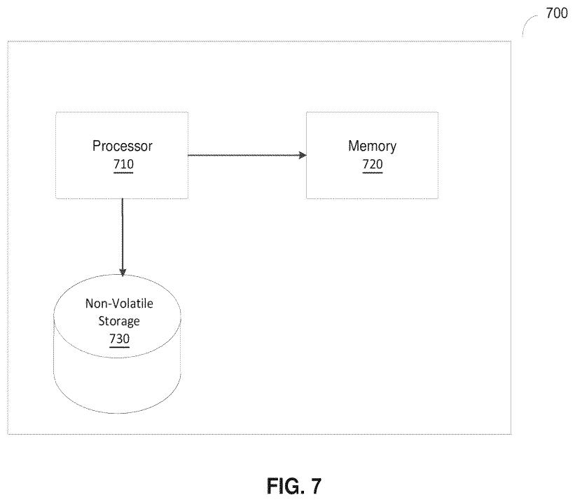

Additionally, an illustrative implementation of a special purpose computer system 700, that may be specially programmed to improve over conventional systems, to be used in connection with any of the embodiments of the disclosure provided herein, is shown in FIG. 7. The computer system 700 may include one or more processors 710 and one or more articles of manufacture that comprise non-transitory computer-readable storage media (e.g., memory 720 and one or more non-volatile storage media 770). The processor 710 may control writing data to and reading data from the memory 720 and the non-volatile storage device 770 in any suitable manner. To perform any of the functionality described herein (e.g., secure execution, proxied execution, sandboxed execution, etc.), the processor 710 may execute one or more processor-executable instructions stored in one or more non-transitory computer-readable storage media (e.g., the memory 720), which may serve as non-transitory computer-readable storage media storing processor-executable instructions for execution by the processor 710. In still other embodiments, the structured data format (e.g., the CPTM) can be implemented as a standalone file that is downloaded/uploaded to any participating systems. The standalone file configures such systems to ensure they are automatically programmed to the meet the data collection and operational needs of a study/protocol associated with the specific CPTM file. Some examples of such systems ideal for such integrations are: CTMS, EDC, TMF, IRTS, IXRS, eCOA/PRO, among other options. The various systems can include: Clinical Trial Management System, software system used by clinical research teams to manage the planning, tracking, and execution of clinical trials. EDC: Electronic Data Capture a system used to collect, manage, and store clinical trial data electronically, as opposed to paper-based methods. TMF: Trial Master File a collection of essential documents for a clinical trial, which are needed to ensure compliance with regulatory requirements and good clinical practice. IRT or IRT System: Interactive Response Technology, a system used for randomization and trial supply management in clinical trials, ensuring that participants are assigned to appropriate treatment groups and that supplies are managed efficiently. IXRS: Interactive Voice Response System a telephone-based system that allows clinical trial participants or clinical staff to interact with trial systems for tasks like randomization, data collection, and monitoring. eCOA: Electronic Clinical Outcome Assessment a tool used to collect patient-reported outcomes (PRO) or other clinical data electronically, usually via a device or app. PRO: Patient-Reported Outcome, data provided directly by the patient about their health condition or treatment without interpretation by a clinician or anyone else.

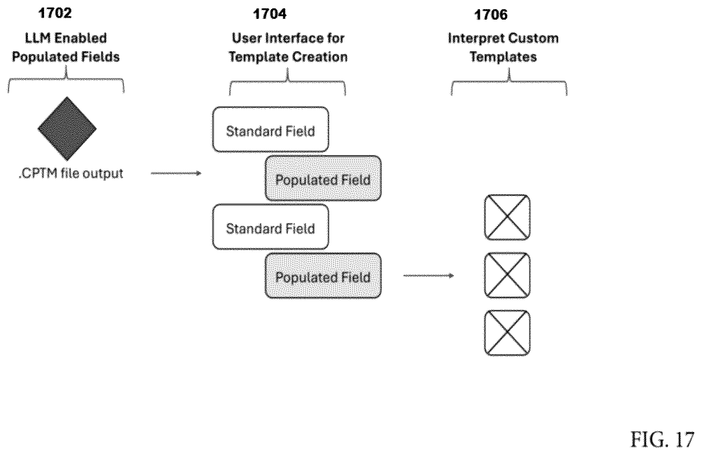

According to various embodiments, the system for data management includes at least one processor operatively connected to a memory, where the at least one processor when executing is configured to accept input of unstructured or multi-format clinical trial data and process the unstructured or multi-format data into a compressed structured data format. As described previously, the compressed structured data format organizes clinical trial protocol information based on procedure-based associations. FIG. 17 illustrates generation of custom templates derived from a data structure including machine learning fields, demonstrating how the system transforms processed clinical trial data into operational templates for clinical trial execution. According to one embodiment, the workflow depicted in FIG. 17 begins with LLM

Enabled Populated Fields, represented as a diamond shape indicating a .CPTM file output. The CPTM file output contains structured data that has been extracted and organized by the machine learning model from unstructured or multi-format clinical trial data sources. The LLM Enabled Populated Fields represent data elements that have been automatically identified, extracted, and formatted by the large language model during the ingestion and transformation process described above.

According to some embodiments, the .CPTM file output feeds into a User Interface for Template Creation. The User Interface for Template Creation contains a series of field elements arranged vertically, where the field elements alternate between Standard Field elements and Populated Field elements. The Standard Field elements represent template fields that require manual input or configuration by clinical trial personnel. The Populated Field elements are shown with shaded backgrounds to distinguish them from the Standard Field elements, and represent fields that have been automatically populated by the large language model processing. According to one example, a Standard Field element can include a blank entry area for site-specific information such as local contact details or institutional identifiers, while a Populated Field element can include automatically extracted protocol information such as dosing schedules, procedure timing windows, or eligibility criteria parameters.

According to another embodiment, the system achieves 90% conversion accuracy for transforming standard text protocols to the CPTM digital format and 95% accuracy for formatting operational tool templates. These accuracy levels enable the system to reliably generate custom templates while maintaining data integrity across clinical trial operations. The distinction between Standard Fields and Populated Fields in the User Interface for Template Creation allows clinical trial personnel to identify which data elements have been automatically extracted and which elements require manual entry or verification.

According to some embodiments, the system uses a human-in-the-loop approach where clinical experts validate AI-generated templates, provide corrections, and highlight areas for improvement. The human-in-the-loop approach ensures that Populated Field elements are verified for accuracy before the templates are deployed for clinical trial execution. According to one example, a clinical research coordinator can review the Populated Field elements displayed in the User Interface for Template Creation, confirm that the automatically extracted procedure schedules match the source protocol document, and make corrections where the machine learning model has produced inaccurate extractions. As the system accumulates feedback from clinical experts, the machine learning model can be refined to improve extraction accuracy, enabling a transition to AI-driven processing based on performance benchmarks.

According to further embodiments, the workflow continues from the User Interface for Template Creation to Interpret Custom Templates. The Interpret Custom Templates portion is represented by a series of placeholder boxes with crossed diagonal lines indicating template output locations. An arrow connects the Populated Field elements to these template placeholders, illustrating how the populated data flows from the user interface into the final interpreted custom templates. The system interprets the combination of Standard Fields and Populated Fields to generate custom templates that are tailored to specific clinical trial protocols, trial sites, or patient contexts.

According to another embodiment, the custom templates generated through this workflow can include treatment checklists, grids, patient calendars, informed consent form drafts, electronic data capture templates, and pharmacy dispensation schedules. Each custom template incorporates the Populated Field elements extracted by the machine learning model while preserving Standard Field elements for site-specific or context-specific data entry. The system's ability to distinguish between these field types enables efficient template generation while maintaining flexibility for clinical trial personnel to customize outputs based on local requirements or patient-specific considerations.

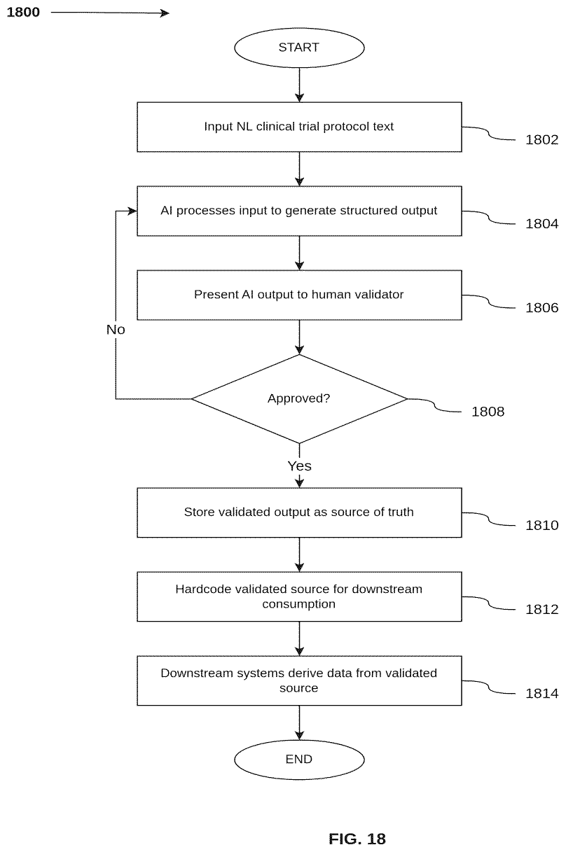

According to various embodiments, the system implements a method 1800 for creating and validating a single source of truth (e.g., using a compressed data format/representation) for clinical trial protocol data using AI with human validation. Referring to FIG. 18, the method 1800 provides a two-tier architecture that separates AI-permitted processing from AI-prohibited processing to eliminate hallucination risks in validated outputs. The two-tier architecture includes an AI-permitted zone where large language model extraction occurs and an AI-prohibited zone where deterministic template instantiation and hardcoded field mapping generate downstream artifacts without any AI inference.

According to one embodiment, the method 1800 begins with a step 1802 of inputting natural language clinical trial protocol text. At step 1802, the system receives unstructured or multi-format clinical trial data as described previously. The natural language clinical trial protocol text can include conventional clinical trial protocol documents spanning hundreds of pages of detailed execution information, dosing instructions, eligibility criteria, procedure schedules, and safety monitoring requirements. The step 1802 represents the entry point into the AI-permitted zone of the two-tier architecture.

According to some embodiments, the method 1800 proceeds to a step 1804 where AI processes the input to generate structured, machine-readable output. At step 1804, the system instantiates a machine learning model trained on clinical protocol data to identify procedure-based information and associated clinical trial data in response to input of unstructured or multi-format clinical trial data. The AI model analyzes the natural language clinical trial protocol text to extract data elements, identify procedures, recognize conditional logic, parse scheduling information, and perform vocabulary normalization. According to one example, the machine learning model can identify dosing thresholds, treatment stopping criteria, and branching conditions from the natural language text and map these elements to structured logical operators in a machine-readable format. The step 1804 outputs the compressed data format including respective procedure, procedure-based associations, and associated clinical trial data. The compressed data format is organized on procedure-based associations linking procedure-based information to associated clinical trial data, as described above.

According to another embodiment, the method 1800 continues to a step 1806 of presenting the AI-generated output to a human validator for review. At step 1806, the user interfaces display the structured, machine-readable output generated by the AI model to a qualified human reviewer. The human reviewer can include a clinical research coordinator, clinical operations specialist, or medical monitor who validates the extracted content against the source protocol document. The step 1806 creates a validation gate that prevents AI hallucinations from propagating downstream into the AI-prohibited zone.

With continued reference to FIG. 18, the method 1800 includes a decision 1808 that determines whether the human validator approves the output. At the decision 1808, the human validator evaluates the accuracy and completeness of the AI-generated structured output against the source natural language clinical trial protocol text. If the human validator does not approve the output at the decision 1808, the method 1800 loops back to the step 1804 for AI reprocessing with corrections or refinements. The loop between the decision 1808 and the step 1804 enables iterative refinement of the AI-generated output until the human validator confirms that the structured data accurately represents the source protocol information.

According to some embodiments, when the human validator approves the output at the decision 1808, the method 1800 continues to a step 1810 of storing the validated output as the authoritative source of truth. At step 1810, the storage component stores the validated structured protocol data object in a locked state. The human validation gate transitions the structured protocol data object from a draft state to the locked state, where the state transition is irreversible. The locked structured protocol data object serves as the architectural boundary between the AI-permitted zone and the AI-prohibited zone. According to one example, once the cptm format data object is locked At step 1810, the cptm format data object cannot be modified without creating a new version that itself requires validation and re-locking through the method 1800.

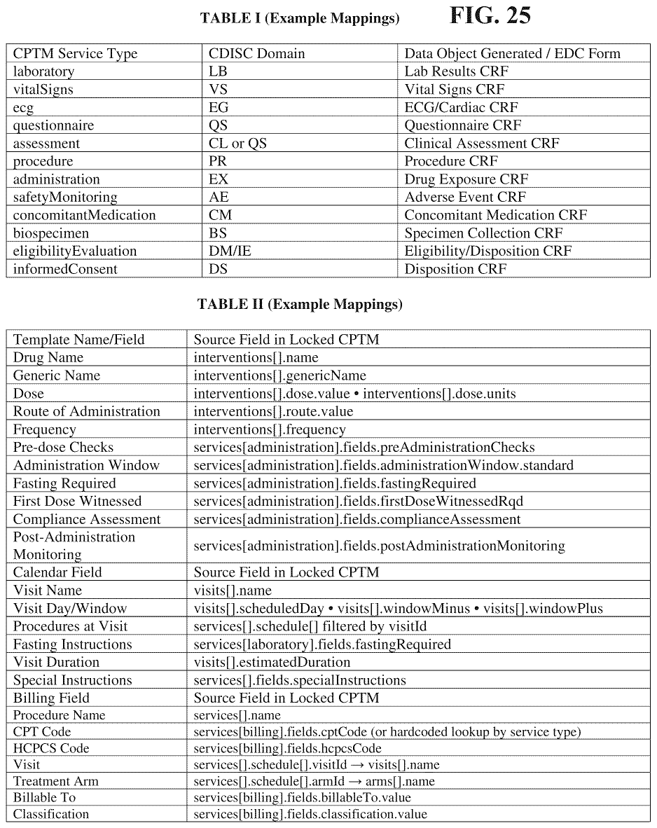

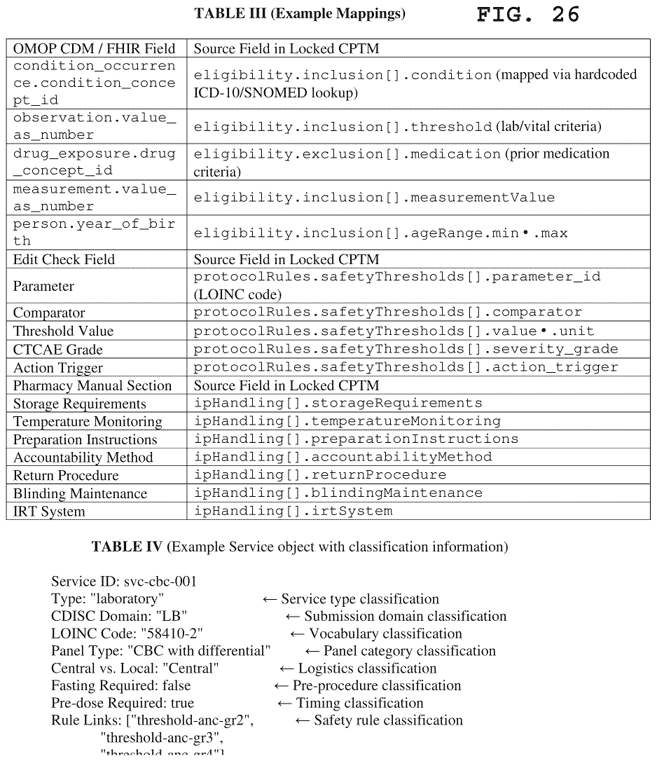

According to further embodiments, the method 1800 proceeds to a step 1812 where the validated source is hardcoded for downstream consumption. At step 1812, the system ensures that all dependent systems reference the verified data stored in the locked structured protocol data object. The step 1812 establishes that everything downstream of the AI is hardcoded to eliminate hallucination risks in validated outputs. The hardcoding At step 1812 means that downstream artifact generation occurs exclusively through deterministic template instantiation and hardcoded field mapping against the validated structured protocol data object, with no AI inference, no neural network processing, and no probabilistic computation in the AI-prohibited zone. According to one embodiment, the hardcoded mappings at Step 1812 invoke a set of static, pre-defined field mapping tables. For each downstream artifact type tables define which CPTM field populates which template placeholder. FIGS. 25-26 includes Tables I, II, and III which described example mappings.

According to another embodiment, the method 1800 includes a step 1814 indicating that downstream systems and documents derive their data exclusively from the validated source. At step 1814, the system generates operational tools including the treatment checklist, grids, patient calendars, the ICF drafts, EDC templates, and the pharmacy dispensation schedules through deterministic generation from the locked structured protocol data object. The step 1814 maintains consistency and accuracy throughout the platform by ensuring that every downstream artifact references the single source of truth validated At step 1810. According to one example, when a protocol amendment changes a procedure in the validated source of truth, the change propagates deterministically to every downstream document that references that procedure, eliminating the manual rework and inconsistency that characterizes conventional approaches.

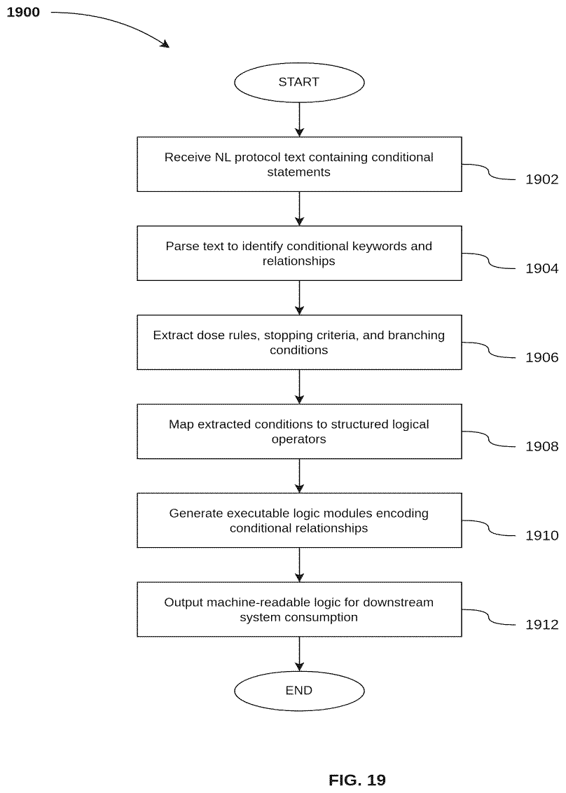

According to various embodiments, the system implements a method 1900 for converting natural language conditional statements from clinical trial protocols into executable machine-readable logic modules. Referring to FIG. 19, the method 1900 provides a systematic approach for transforming conditional logic expressed in natural language protocol text into structured logical operators that downstream systems can consume directly without manual hardcoding. The method 1900 enables the system to process unstructured or multi-format clinical trial data containing conditional statements such as dose thresholds, treatment rules, and safety criteria, and output machine-readable executable modules that encode the conditional relationships identified in the protocol.

According to one embodiment, the method 1900 begins with a step 1902 of receiving natural language protocol text containing conditional statements such as dose thresholds and treatment rules. At step 1902, the system accepts input of unstructured or multi-format clinical trial data as described previously. The natural language protocol text can include conditional statements expressing dose adjustment rules, treatment stopping criteria, safety monitoring thresholds, and branching conditions. According to one example, the natural language protocol text can include statements such as “if blood level exceeds 5.0, stop treatment” or “administer 20 mg oral dose daily, reduce to 10 mg if ALT exceeds 3 times the upper limit of normal.” The step 1902 receives these conditional statements in their original natural language form from the clinical trial protocol data.

According to some embodiments, the method 1900 proceeds to a step 1904 of parsing the text to identify conditional keywords and relationships including if/then constructs, and/or logical operators, and threshold values. At step 1904, the system executes a plurality of rules to identify procedure-based information in unstructured or a plurality of non-homogeneous data sources including unstructured text, unformatted text, or natural language data sources. The parsing At step 1904 identifies conditional keywords such as “if,” “then,” “when,” “unless,” and “provided that” within the natural language protocol text. The step 1904 also identifies logical operators including AND, OR, and NOT that define relationships between multiple conditions. According to one example, the parsing At step 1904 can identify threshold values expressed as numeric comparisons such as “greater than 5 times ULN” or “less than 1000/mm³” and associate these threshold values with the corresponding clinical parameters referenced in the conditional statement.

According to another embodiment, the method 1900 continues to a step 1906 of extracting dose adjustment rules, treatment stopping criteria, and branching conditions from the parsed text. At step 1906, the system extracts specific categories of conditional logic that govern clinical trial execution. The dose adjustment rules extracted At step 1906 can include instructions for modifying dosing based on patient response or laboratory values. The treatment stopping criteria extracted At step 1906 can include discontinuation conditions that specify when treatment should be halted based on safety signals or clinical events. The branching conditions extracted At step 1906 can include decision points that determine different treatment pathways based on patient characteristics or clinical measurements. According to one example, the step 1906 can extract a dose adjustment rule specifying that the dose should be reduced by one level if ALT is between 3 and 5 times ULN with normal bilirubin, and a treatment stopping criterion specifying that treatment should be discontinued if ALT exceeds 5 times ULN.

In one example, 1904 is executed within an AI-permitted zone by a large language model operating under a structured extraction system prompt. The parsing occurs as follows: (a) input to the parsing step: Full protocol document text (extracted from PDF, e.g., 100-400 pages), a CPTM protocolRules schema specification (e.g., including the THRESHOLD_TYPES and CRITERIA_OBJECT_SHAPE definitions, and structured extraction instructions specifying the target output format. The parsing mechanism identifies four categories of linguistic patterns: (A) Conditional keywords and if/then constructs, where the system prompt instructs the LLM to identify sentences containing the following trigger expressions: “if”, “when”, “in the event that”, “provided that”, “unless”, “should”, “upon”, “in case of”, “following”, “after confirmation of”. Each identified conditional sentence is a candidate for extraction as a criteria object. Also extracted are discontinuation criterion with criteria.logic: “AND”, two condition nodes, and action.type: “discontinue”; (B) Logical operators: the system identifies explicit logical connectors—“AND”, “OR”, “AND/OR”, “NOT”, “neither”, “either”—and maps them to the criteria.logic field (“AND” |“OR” |“SINGLE”) and to the operator field in AESI logic tree nodes. Nested logical structures (e.g., “A AND (B OR C)”) are extracted into criteria. groups[ ] with nested condition arrays; (C) Threshold values: numerical patterns are parsed and classified into one of the three THRESHOLD_TYPES: (i) relative thresholds (e.g., “3×ULN”, “2 times the upper limit of normal”, “5×ULN”): {type: “relative”, multiplier: 3, reference: “ULN”}; absolute thresholds (e.g., “ANC <500/mm³”, “eGFR <60 mL/min/1.73 m²”): {type: “absolute”, value: 500, units: “/mm³”}; and change-from-baseline thresholds (e.g., “QTcF increase >60 ms from baseline”, “lymphocyte count decreased ≥30% from baseline”): {type: “change”, direction: “increase”, value: 60, units: “ms”, reference: “baseline”} where comparators are extracted and normalized: “greater than”→“>”, “at least”→“>=”, “less than”→“<”, “no more than”→“<=”, “equal to”→“=”; and (D) action keywords: following the condition clause, the system identifies the required clinical action: “hold”, “discontinue”, “reduce dose”, “delay”, “rechallenge”, “notify”, “monitor”, “report”. These map to the structured CRITERIA_ACTION_SCHEMA types (hold, discontinue, modify, delay, rechallenge).

Example output produced: A CRITERIA_OBJECT_SHAPE-conforming JSON object per extracted rule, with populated fields for criteria.logic, criteria.conditions[ ] (each with test, operator, threshold), action (typed structured object), and followUp. This output feeds directly into the protocolRules section of the draft CPTM object.

The system handles natural language variability, and protocols from different sponsors use different phrasing to generate the same logical condition. The system normalizes these to the structured schema.

With continued reference to FIG. 19, the method 1900 includes a step 1908 of mapping the extracted conditions to structured logical operators in a machine-readable format. At step 1908, the system transforms the extracted conditional logic into structured data representations that conform to the structured data format described previously. The structured data format includes a protocolRules section comprising machine-readable objects for AESI threshold definitions, concomitant therapy rule arrays with allowed/prohibited/restricted classifications and washout periods, discontinuation criteria as evaluatable condition trees, follow-up rule specifications, and reproductive safety requirement objects with action-on-positive logic. According to some embodiments, the mapping At step 1908 encodes AESI definitions as Boolean logic tree data structures. The Boolean logic tree data structures include nodes containing operator fields for AND, OR, and NOT operations. The nodes also contain parameter reference fields identifying clinical measurements by LOINC code or other external vocabulary standards. The nodes further contain comparator fields specifying comparison operations such as greater than, less than, equals, or between. The nodes include threshold fields with numeric values and units that define the boundary conditions for the comparison. The nodes can include child node arrays enabling arbitrary nesting depth to represent complex multi-criteria conditions. According to one example, a Hy's Law criterion can be encoded as a Boolean logic tree with an OR operator at the root node, where one child node specifies ALT greater than 5 times ULN, and another child node contains an AND operator with child nodes specifying ALT greater than 3 times ULN and total bilirubin greater than 2 times ULN. For example, the method and/or systems implementing the method can be configured to match specific patient/care recipients to safety criterion not explicitly specified, and encode that safety criterion as executable language into data structures associated with that specific patient/care recipient. The system and method provide enhanced safety over known approached, and leverage encoding of those safety parameters into data structures that propagated those parameters to downstream systems and/or processes.

According to further embodiments, the mapping At step 1908 generates safety threshold objects that encode graded severity monitoring criteria. The safety threshold objects contain parameter identifiers conforming to external vocabulary standards such as LOINC codes. The safety threshold objects contain comparators, values, and units that define the threshold boundary. The safety threshold objects contain action triggers specifying responses such as hold_treatment, notify_PI, dose_reduce, or discontinue. The safety threshold objects contain severity grades from 1 to 4 per CTCAE or protocol-specific grading scales. According to one example, an absolute neutrophil count parameter can be associated with multiple safety threshold objects: a Grade 2 threshold object specifying ANC less than 1.5×10⁹/L with action trigger of monitor, a Grade 3 threshold object specifying ANC less than 1.0×10⁹/L with action trigger of hold_treatment, and a Grade 4 threshold object specifying ANC less than 0.5×10⁹/L with action trigger of discontinue.

According to another embodiment, the method 1900 proceeds to a step 1910 of generating executable logic modules that encode the conditional relationships identified in the protocol. At step 1910, the system produces machine-readable modules that can be evaluated computationally against patient clinical data. The executable logic modules generated At step 1910 encode the Boolean logic trees, safety threshold objects, concomitant therapy rule arrays, discontinuation criteria condition trees, follow-up rule specifications, and reproductive safety requirement objects mapped At step 1908. The executable logic modules enable automated evaluation of protocol safety rules against incoming patient data without manual interpretation by clinical staff at each participating site.

According to some embodiments, the method 1900 concludes with a step 1912 of outputting the machine-readable conditional logic for consumption by downstream systems. At step 1912, the system outputs the executable logic modules in a format that enables programmatic enforcement of protocol rules without manual hardcoding. The machine-readable conditional logic output At step 1912 can be consumed by downstream systems to generate safety monitoring configurations, automated threshold alerts, dose modification recommendations, and discontinuation triggers. The step 1912 enables downstream systems to extrapolate the conditional logic information directly from the structured data format rather than requiring each downstream system to have the conditional logic manually hardcoded or adjusted to be executable by the downstream systems. According to one example, the machine-readable conditional logic output At step 1912 can be consumed by an electronic data capture system to automatically configure edit checks and validation rules, or by a clinical trial management system to generate safety monitoring dashboards that evaluate patient data against the encoded threshold objects. In further example, the compressed data format (e.g., node-based data structure) associated with each patient/care recipient provides reduced storage requirements for complex data, integrates executable logic executable by downstream systems, and eliminates data formatting and mapping errors from which conventional implementation suffers.

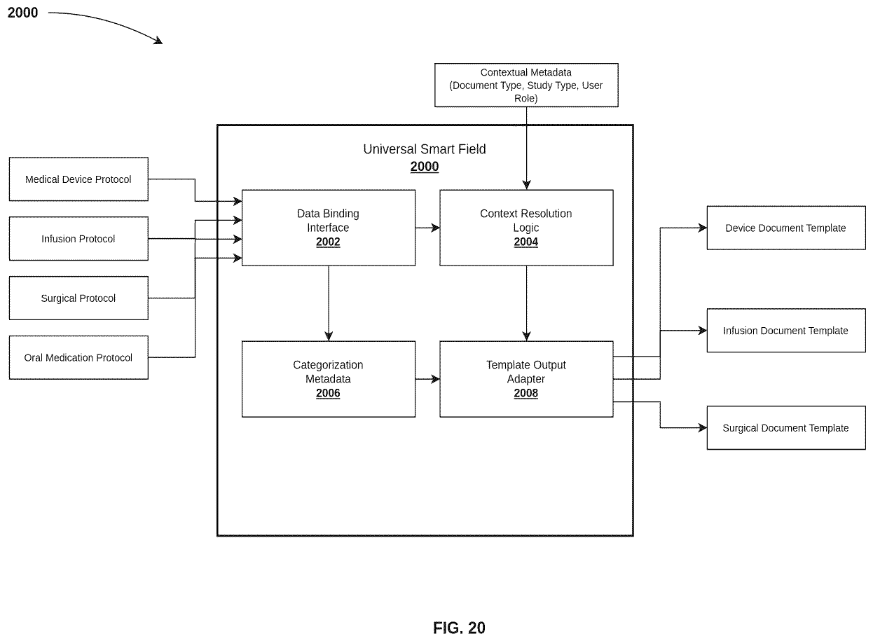

According to various embodiments, the system implements a universal smart field 2000 that serves as an intermediary layer for resolving data from the structured data format across different document types and user contexts. Referring to FIG. 20, the universal smart field 2000 provides a flexible architecture that can hydrate any template while being robust enough to fill conditional logic or conditional smart forms on the output side based on context metadata awareness of the user type. The universal smart field 2000 enables a single smart field to be recycled across different study types, including medical device protocols, infusion protocols, and surgical protocols, providing accurate information based on categorization and context of the requesting user.

According to one embodiment, the universal smart field 2000 includes a data binding interface 2002 that receives input data from multiple protocol types. As shown in FIG. 20, the data binding interface 2002 accepts input from Medical Device Protocol, Infusion Protocol, Surgical Protocol, and Oral Medication Protocol sources. The data binding interface 2002 serves as an entry point that aggregates diverse protocol inputs into the universal smart field 2000 regardless of the originating protocol type. According to one example, the data binding interface 2002 can receive intervention data from a medical device study specifying device model and implantation parameters, and the same data binding interface 2002 can receive intervention data from an infusion study specifying drug concentration and infusion duration. The data binding interface 2002 normalizes these diverse inputs for processing by subsequent components within the universal smart field 2000.

According to some embodiments, the universal smart field 2000 includes a context resolution logic module 2004 that receives contextual metadata and determines the appropriate data representation based on these contextual factors. The context resolution logic 2004 receives contextual metadata such as document type, study type, and user role from the requesting system or user. The context resolution logic 2004 evaluates the contextual metadata to determine how the data bound through the data binding interface 2002 should be represented for the specific requesting context. According to one example, when the context resolution logic 2004 receives contextual metadata indicating that the requesting user is a surgeon preparing a surgical protocol document, the context resolution logic 2004 determines that intervention data should be represented with surgical procedure specifications and device dimensions. When the context resolution logic 2004 receives contextual metadata indicating that the requesting user is a patient receiving treatment instructions, the context resolution logic 2004 determines that the same intervention data should be represented with simplified dosing schedules and administration timing.

With continued reference to FIG. 20, the universal smart field 2000 includes a categorization metadata component 2006 that stores classification information about the data type and study type. The categorization metadata 2006 enables protocol-agnostic categorization by maintaining classification information that allows the universal smart field 2000 to process data from any protocol type through a common categorization scheme. The categorization metadata 2006 stores information indicating whether bound data represents a medical device intervention, an infusion intervention, a surgical intervention, or an oral medication intervention. The categorization metadata 2006 enables the universal smart field 2000 to apply appropriate processing rules based on the category of the bound data regardless of the specific protocol from which the data originated. According to one example, the categorization metadata 2006 can classify an intervention as a device-based intervention, enabling the context resolution logic 2004 to apply device-specific resolution rules when generating output for device document templates.

According to another embodiment, the universal smart field 2000 includes a template output adapter 2008 that formats the resolved data for specific downstream templates. As shown in FIG. 20, the template output adapter 2008 produces appropriately formatted outputs to Device Document Templates, Infusion Document Templates, and Surgical Document Templates. The template output adapter 2008 receives resolved data from the context resolution logic 2004 and the categorization metadata 2006, and formats the resolved data according to the requirements of the target template type. The template output adapter 2008 enables the universal smart field 2000 to act as a wide funnel accepting diverse protocol inputs through the data binding interface 2002 while producing context-appropriate outputs for each document template type.

According to some embodiments, the data binding interface 2002 feeds both the context resolution logic 2004 and the categorization metadata 2006, while both modules feed into the template output adapter 2008. This architecture enables the universal smart field 2000 to simultaneously consider the contextual factors evaluated by the context resolution logic 2004 and the classification information stored in the categorization metadata 2006 when generating formatted output through the template output adapter 2008. According to one example, when a user requests an intervention field for a surgical protocol document, the data binding interface 2002 receives the intervention data, the context resolution logic 2004 determines that surgical procedure details are appropriate based on the document type metadata, the categorization metadata 2006 confirms the intervention is categorized as a surgical intervention, and the template output adapter 2008 formats the resolved data with surgical specifications for the Surgical Document Template.

According to further embodiments, the universal smart field 2000 architecture supports the execution of the plurality of rules that include mapping identified procedures to associated clinical trial data as described previously. The context resolution logic 2004 applies rules to map procedure data bound through the data binding interface 2002 to the appropriate clinical trial data representation based on the contextual metadata. The categorization metadata 2006 stores the classification information that enables the rules to identify which procedure category applies to the bound data. The template output adapter 2008 then maps the identified procedures to the associated clinical trial data fields required by the target template. According to one example, when the universal smart field 2000 processes a laboratory procedure, the context resolution logic 2004 applies rules to determine whether the laboratory procedure should be mapped to specimen collection instructions for a site coordinator or to result interpretation guidelines for a medical monitor, based on the user role specified in the contextual metadata.

FIG. 26 illustrates example data, and for example Table 4 is an example data object. This classification information is consumed differently by each downstream artifact. For example, EDC form generation: uses cdiscDomain: “LB” to assign the correct submission domain; uses loincCode to set the variable name; uses ruleLinks to generate edit checks for ANC grading; Patient calendar: uses schedule[ ] to place the lab at the correct visits; uses preDoseRequired: true to add a sequencing constraint (“before study drug”); Treatment checklist: uses ruleLinks to generate the ANC threshold monitoring instructions adjacent to the drug administration step; Billing grid: uses type: “laboratory”+panelType to look up the CPT code (85025 for CBC with differential); ICF: uses type: “laboratory”+visit count from schedule[ ] to populate the blood draw frequency and total volume section of the informed consent. In each case, the context resolution logic reads the classification information from the same locked procedure node and selects the appropriate rendering, with no AI inference involved.

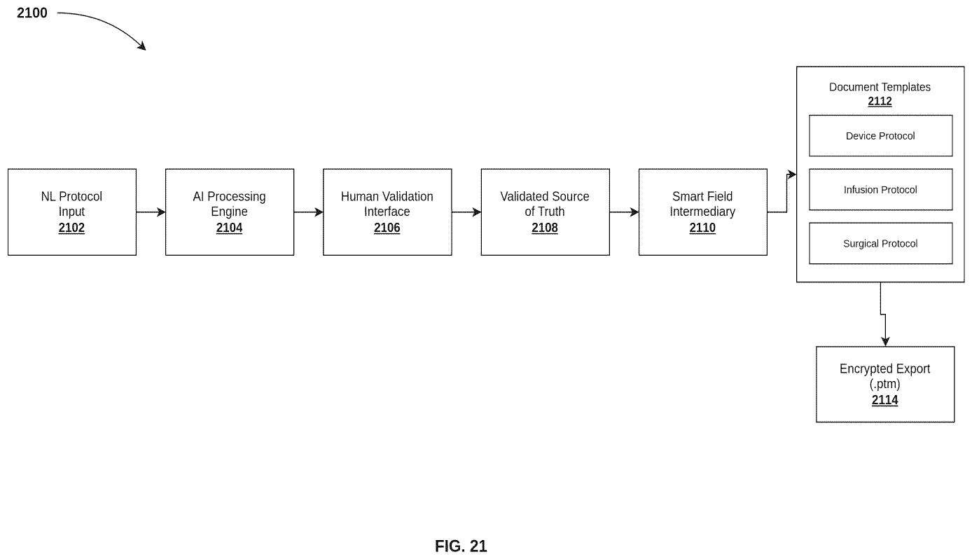

According to various embodiments, the system implements a clinical trial protocol processing platform 2100 that provides an end-to-end architecture for transforming natural language protocol text into validated structured data and generating operational documents through deterministic processing. Referring to FIG. 21, the platform 2100 includes a plurality of interconnected components that collectively implement the two-tier architecture separating AI-permitted processing from AI-prohibited processing as described previously. The platform 2100 receives natural language protocol input, processes the input through AI extraction with human validation, stores validated data as an authoritative reference, and generates operational documents through a smart field intermediary layer with encrypted export capabilities.

According to one embodiment, the platform 2100 includes an NL protocol input module 2102 that serves as the entry point for receiving natural language clinical trial protocol text. The NL protocol input 2102 accepts unstructured or multi-format clinical trial data, including conventional clinical trial protocol documents containing detailed execution information, dosing instructions, eligibility criteria, procedure schedules, and safety monitoring requirements. The NL protocol input 2102 feeds the received natural language protocol text into subsequent processing components within the platform 2100. According to one example, the NL protocol input 2102 can receive a clinical trial protocol document spanning hundreds of pages containing natural language descriptions of treatment arms, visit schedules, laboratory assessments, and adverse event reporting requirements.

According to some embodiments, the platform 2100 includes an AI processing engine 2104 that performs natural language processing and conditional logic extraction on the protocol text received through the NL protocol input 2102. The AI processing engine 2104 instantiates a machine learning model trained on clinical protocol data to identify procedure-based information and associated clinical trial data in response to input of unstructured or multi-format clinical trial data. The AI processing engine 2104 analyzes the natural language protocol text to extract data elements, identify procedures, recognize conditional logic, including dose thresholds and treatment rules, parse scheduling information, and perform vocabulary normalization. The AI processing engine 2104 outputs structured data to subsequent validation components within the platform 2100. According to one example, the AI processing engine 2104 can extract conditional statements such as “if blood level exceeds 5.0, stop treatment” and map these statements to structured logical operators in a machine-readable format as described previously with reference to the method 1900.

According to another embodiment, the platform 2100 includes a human validation interface 2106 that receives the structured data output from the AI processing engine 2104 and presents the structured data to a human reviewer for confirmation. The human validation interface 2106 enables a qualified human reviewer to validate the extracted content against the source protocol document, confirming accuracy and eliminating AI hallucination risk. The human validation interface 2106 implements the validation gate described previously with reference to the method 1800, preventing AI hallucinations from propagating downstream into the AI-prohibited zone of the platform 2100. According to one example, the human validation interface 2106 can display extracted procedure schedules, dosing parameters, and conditional logic alongside the source natural language text, enabling a clinical research coordinator to verify that the AI processing engine 2104 has accurately extracted the protocol information.

With continued reference to FIG. 21, the platform 2100 includes a validated source of truth 2108 that stores validated data from the human validation interface 2106 and serves as the authoritative hardcoded reference for all downstream systems. The validated source of truth 2108 stores the structured protocol data object in a locked state after human validation approval. The validated source of truth 2108 serves as the architectural boundary between the AI-permitted zone and the AI-prohibited zone within the platform 2100. Everything downstream of the validated source of truth 2108 is hardcoded, ensuring that all dependent systems reference verified data without any AI inference, neural network processing, or probabilistic computation. According to one example, once the cptm format data object is stored in the validated source of truth 2108, the cptm format data object cannot be modified without creating a new version that requires validation through the human validation interface 2106.

According to some embodiments, the platform 2100 includes a smart field intermediary 2110 that connects to the validated source of truth 2108 and serves as a universal, reusable intermediary layer for resolving data across different document types and user contexts. The smart field intermediary 2110 implements the universal smart field 2000 architecture described previously, including the data binding interface 2002, the context resolution logic 2004, the categorization metadata 2006, and the template output adapter 2008. The smart field intermediary 2110 receives requests from document templates and resolves data from the validated source of truth 2108 based on contextual metadata, including document type, study type, and user role. The smart field intermediary 2110 enables a single smart field to be recycled across different protocol types while providing context-appropriate data representations for each requesting template.

According to further embodiments, the platform 2100 includes document templates 2112 that receive resolved data from the smart field intermediary 2110 to generate operational documents. As shown in FIG. 21, the document templates 2112 include Device Protocol templates, Infusion Protocol templates, and Surgical Protocol templates. The document templates 2112 invoke smart fields within the smart field intermediary 2110 to populate template fields with data from the validated source of truth 2108. The document templates 2112 generate operational tools through deterministic template instantiation and hardcoded field mapping against the validated structured protocol data object. According to one example, the document templates 2112 can generate treatment checklists, grids with CPT/HCPCS code mappings, patient visit calendars with window calculations, informed consent documents with protocol-specific language inserted into institutional templates, EDC configurations and CRF form builds including field names, validation rules, edit checks, and skip logic, dose modification tables, regulatory submission templates, pharmacy dispensation schedules, and eligibility criteria mapped to EMR/EHR structured query fields for automated patient pre-screening.

According to another embodiment, the platform 2100 includes an encrypted export module 2114 that processes generated documents for secure distribution using a proprietary encrypted file format. The encrypted export 2114 applies encryption to document content generated through the document templates 2112 and converts the encrypted document into a proprietary.cptm file format. The encrypted export 2114 provides encryption in transit through a transit security layer that delivers encrypted documents to authorized recipients. The system exports data in a fully encrypted format with encryption in transit, using the proprietary.cptm extension that is accessible only to authorized recipients. According to one example, the encrypted export 2114 can process a completed treatment checklist generated from the document templates 2112, apply encryption using the encryption algorithm, convert the encrypted document to the .cptm format, and transmit the encrypted .cptm output through the transit security layer to an authorized clinical trial site while blocking unauthorized access attempts from accessing the encrypted content.

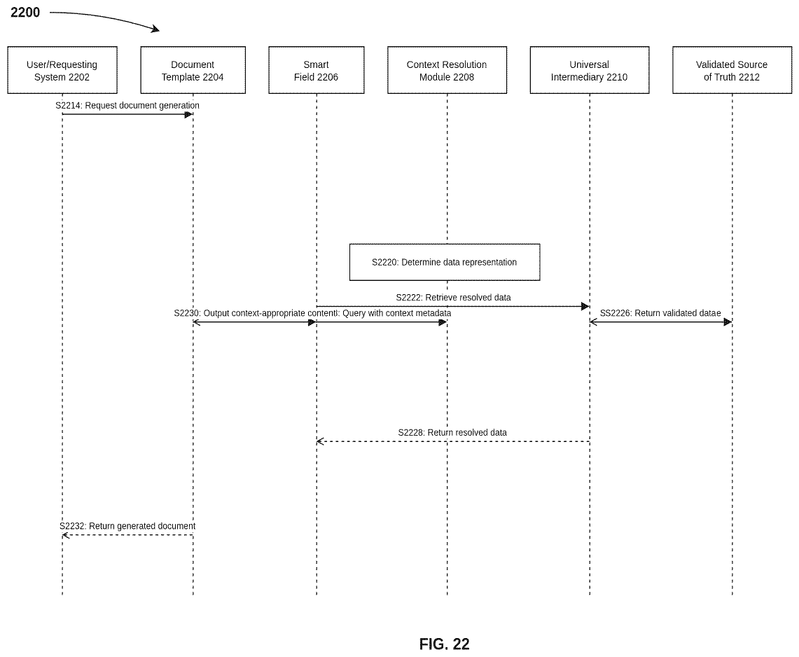

According to various embodiments, the system implements a process 2200 for context-aware resolution of smart fields across different document types and user contexts. Referring to FIG. 22, the process 2200 is illustrated as a sequence diagram showing interactions between six participants that collectively enable the smart field intermediary to resolve data from the validated source of truth 2108 based on contextual metadata. The process 2200 demonstrates how a single smart field can be recycled across different protocol types and user contexts while providing accurate, context-appropriate data representations for each requesting template.

According to one embodiment, the process 2200 involves a user/requesting system 2202, a document template 2204, a smart field 2206, a context resolution module 2208, a universal intermediary 2210, and the validated source of truth 2212. The user/requesting system 2202 initiates the process 2200 by requesting document generation with context metadata. The document template 2204 represents one of the document templates 2112 described previously that invokes smart fields to populate template fields. The smart field 2206 implements the universal smart field 2000 architecture, including the data binding interface 2002, the context resolution logic 2004, the categorization metadata 2006, and the template output adapter 2008. The context resolution module 2208 determines the appropriate data representation based on contextual factors received from the smart field 2206. The universal intermediary 2210 serves as the intermediary layer that retrieves resolved data from the validated source of truth 2212. The validated source of truth 2212 stores the validated structured protocol data object in a locked state as described previously.

According to some embodiments, the process 2200 begins at a step S2214 where the user/requesting system 2202 requests document generation with context metadata including document type, study type, and user role. At step S2214, the user/requesting system 2202 provides contextual metadata that enables subsequent components to determine the appropriate data representation for the requested document. The context metadata can include document type indicators specifying whether the requested document is a treatment checklist, a grid, a patient calendar, an informed consent form draft, an EDC template, or a pharmacy dispensation schedule. The context metadata can include study type indicators specifying whether the study involves a medical device protocol, an infusion protocol, a surgical protocol, or an oral medication protocol. The context metadata can include user role indicators specifying whether the requesting user is a site coordinator, a medical monitor, a patient, or a regulatory specialist. According to one example, the step S2214 can include a request from a site coordinator for a treatment checklist document for a medical device study, where the context metadata specifies document type as treatment checklist, study type as medical device, and user role as site coordinator.

According to another embodiment, the process 2200 proceeds to a step S2216 where the document template 2204 invokes the smart field 2206 such as an intervention field. At step S2216, the document template 2204 encounters a smart field placeholder within the template structure and invokes the smart field 2206 to resolve the data for that placeholder. The smart field 2206 invoked At step S2216 can represent any of the 14 defined service types in the service type taxonomy including administration, assessment, ECG, vital signs, laboratory, imaging, biospecimen, informed consent, questionnaire, procedure, concomitant medication, safety monitoring, randomization, and eligibility evaluation. According to one example, the document template 2204 can invoke an intervention smart field that resolves to different data representations depending on whether the intervention is a medical device, an infusion, a surgical procedure, or an oral medication.

With continued reference to FIG. 22, the process 2200 continues to a step S2218 where the smart field 2206 queries the context resolution module 2208 with the contextual metadata received from the user/requesting system 2202. At step S2218, the smart field 2206 passes the document type, study type, and user role metadata to the context resolution module 2208 for evaluation. The context resolution module 2208 receives the contextual metadata and prepares to determine the appropriate data representation based on the combination of contextual factors. According to one example, the smart field 2206 can query the context resolution module 2208 with metadata indicating that the requesting context is a surgical protocol document for a surgeon user, enabling the context resolution module 2208 to determine that intervention data should include surgical procedure specifications and device dimensions.

According to some embodiments, the process 2200 proceeds to a step S2220 where the context resolution module 2208 determines the appropriate data representation based on the context. At step S2220, the context resolution module 2208 evaluates the contextual metadata received At step S2218 and applies resolution rules to determine how data from the validated source of truth 2212 should be represented for the specific requesting context. The context resolution module 2208 considers the document type to determine which data fields are relevant for the requested document. The context resolution module 2208 considers the study type to determine which protocol-specific parameters apply. The context resolution module 2208 considers the user role to determine the appropriate level of detail and format for the data representation. According to one example, when the context resolution module 2208 receives metadata indicating a patient user requesting treatment instructions, the context resolution module 2208 determines that intervention data should be represented with simplified dosing schedules and administration timing rather than detailed clinical specifications.

According to further embodiments, the process 2200 continues to a step S2222 where the smart field 2206 retrieves resolved data from the universal intermediary 2210. At step S2222, the smart field 2206 requests data from the universal intermediary 2210 based on the data representation determination made by the context resolution module 2208 At step S2220. The universal intermediary 2210 serves as the intermediary layer that connects the smart field 2206 to the validated source of truth 2212. The universal intermediary 2210 receives the data retrieval request from the smart field 2206 and prepares to access the validated source of truth 2212 to obtain the requested data. According to one example, the smart field 2206 can request intervention data from the universal intermediary 2210 with instructions to retrieve device specifications for a medical device intervention based on the context resolution determination.

According to another embodiment, the process 2200 proceeds to a step S2224 where the universal intermediary 2210 accesses the validated source of truth 2212. At step S2224, the universal intermediary 2210 retrieves data from the validated source of truth 2212 that stores the validated structured protocol data object in a locked state. The validated source of truth 2212 contains the cptm format data object including procedure-based associations linking procedure-based information to associated clinical trial data. The data retrieved At step S2224 can include clinical procedure objects that serve as the primary organizing entity simultaneously carrying schedule linkage, multi-vocabulary terminology alignment including LOINC, CDISC, NIH CDE, and SNOMED identifiers, protocolRules references, cross-reference index, and downstream generation anchoring. A single service object retrieved from the validated source of truth 2212 simultaneously carries vocabulary identifiers from multiple independent clinical standards including LOINC, CDISC domain, NIH CDE, and SNOMED alongside protocol-specific operational parameters. According to one example, the universal intermediary 2210 can retrieve a laboratory service object from the validated source of truth 2212 that carries a LOINC code identifying the specific laboratory test, a CDISC domain tag for SDTM mapping, an NIH CDE reference for cross-study harmonization, and protocol-specific parameters including timing windows and threshold values.

With continued reference to FIG. 22, the process 2200 includes return messages flowing back through the participant chain. At a step S2226, validated data returns from the validated source of truth 2212 to the universal intermediary 2210. The validated data returned At step S2226 includes the structured protocol data retrieved At step S2224 with all associated vocabulary identifiers and protocol-specific parameters. At a step S2228, resolved data returns from the universal intermediary 2210 to the smart field 2206. The resolved data returned At step S2228 includes the validated data formatted according to the data representation determination made by the context resolution module 2208. At a step S2230, context-appropriate content is output from the smart field 2206 to the document template 2204. The context-appropriate content output At step S2230 includes the resolved data formatted for insertion into the specific template field that invoked the smart field 2206 At step S2216. At a step S2232, the generated document is returned from the document template 2204 to the user/requesting system 2202. The generated document returned At step S2232 includes the completed template with all smart field placeholders populated with context-appropriate content derived from the validated source of truth 2212.

According to some embodiments, the process 2200 enables the system to generate operational documents that maintain consistency with the validated source of truth 2212 while providing context-appropriate data representations for different document types, study types, and user roles. The process 2200 ensures that all generated documents derive data exclusively from the validated source of truth 2212 through deterministic resolution without any AI inference at the document generation step. According to one example, the same intervention smart field can be invoked by different document templates 2112 for different user contexts, and the process 2200 ensures that each invocation retrieves the same validated data from the validated source of truth 2212 while formatting the data appropriately for the specific requesting context based on the contextual metadata provided At step S2214.

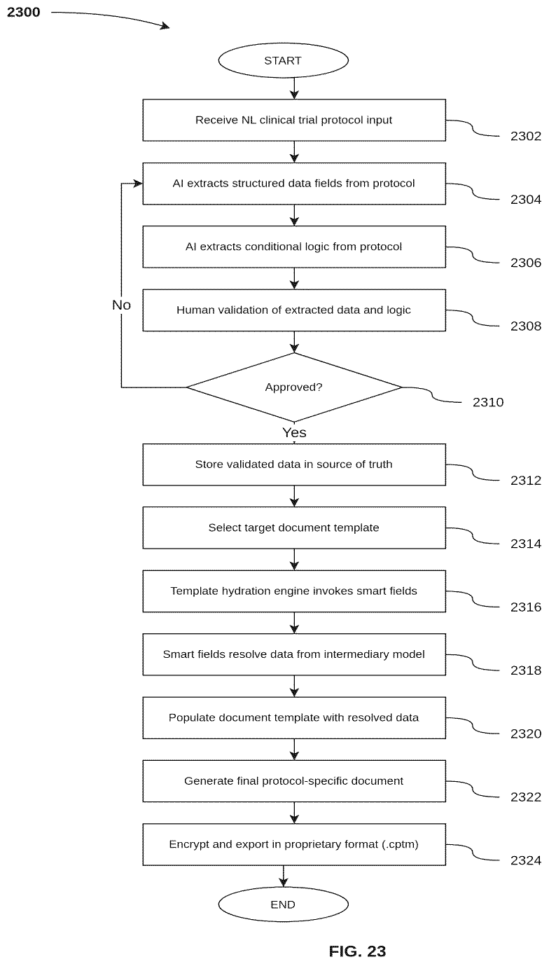

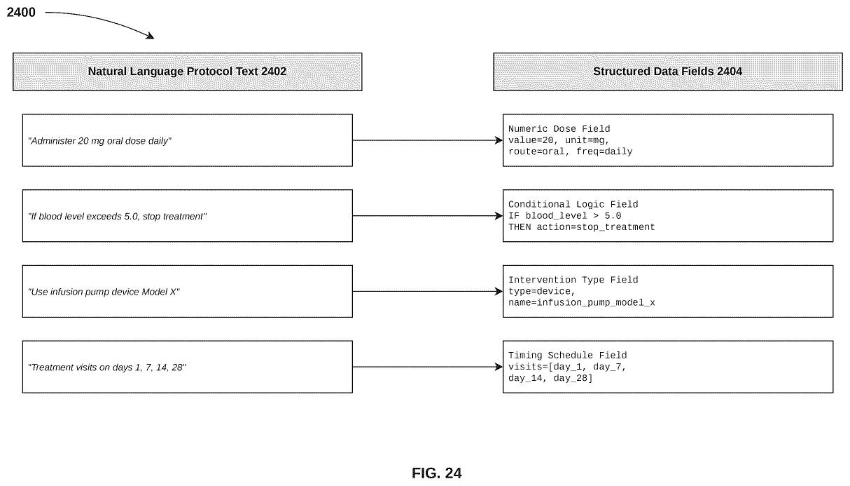

According to various embodiments, the system implements a method 2300 for end-to-end generation of clinical trial documents from source protocol text. Referring to FIG. 23, the method 2300 provides a comprehensive workflow that integrates AI extraction, human validation, smart field resolution, and encrypted export to transform natural language clinical trial protocol input into protocol-specific operational documents. The method 2300 combines the AI-permitted processing zone, the validation gate, the deterministic generation zone, and the encrypted export capabilities described previously into a unified end-to-end process flow.

According to one embodiment, the method 2300 begins with a step 2302 of receiving natural language clinical trial protocol input. At step 2302, the system accepts unstructured or multi-format clinical trial data including conventional clinical trial protocol documents containing detailed execution information. The natural language clinical trial protocol input received At step 2302 can include dosing instructions, eligibility criteria, procedure schedules, safety monitoring requirements, concomitant therapy restrictions, discontinuation criteria, follow-up rules, and reproductive safety requirements expressed in natural language form.

According to one example, the step 2302 can receive a clinical trial protocol document containing natural language descriptions of concomitant therapy rule arrays that encode medication class restrictions with medication class identifiers such as ATC codes, status classifications including allowed, prohibited, restricted, and requires\_washout designations, washout period durations, condition arrays specifying when restrictions apply, and exception arrays specifying when restrictions do not apply.

According to some embodiments, the method 2300 proceeds to a step 2304 where AI extracts structured data fields from the protocol. At step 2304, the AI processing engine 2104 instantiates a machine learning model trained on clinical protocol data to identify procedure-based information and associated clinical trial data from the natural language input received At step 2302. The step 2304 extracts data elements including procedure definitions, visit schedules, dosing parameters, eligibility criteria, and intervention specifications. The system implements a three-layer decomposition of interventional entities At step 2304, extracting definition layer data specifying what the intervention is, logistics layer data specifying how the intervention is managed and moved, and task layer data specifying what actually happens at a visit, with explicit referential links between layers. According to one example, the step 2304 can extract structured data fields for a drug intervention including definition layer fields for drug identity, formulation, and dosing specifications, logistics layer fields for investigational product handling, storage requirements, and shipping parameters, and task layer fields for scheduled administration activities at specific visits.

According to another embodiment, the method 2300 continues to a step 2306 where AI extracts conditional logic from the protocol. At step 2306, the AI processing engine 2104 identifies and extracts conditional statements including dose thresholds, treatment rules, safety criteria, and branching conditions from the natural language protocol text. The step 2306 extracts discontinuation criteria encoded as evaluatable condition trees with criterion type enumerations including investigator\_decision, protocol\_specified, sponsor\_directed, and patient\_withdrawal classifications, action required arrays specifying procedural steps upon discontinuation, and follow-up plan references defining post-discontinuation visit requirements. The step 2306 also extracts follow-up rules that specify post-event clinical obligations as structured objects containing trigger event references, follow-up type classifications, schedule window arrays, required service arrays, and termination condition trees specifying when follow-up ends. According to one example, the step 2306 can extract reproductive safety requirements encoded as structured objects containing affected sex fields, contraception requirement arrays with method type, duration, and start timing, pregnancy test schedules specifying test type, frequency, and timing relative to dosing, action-on-positive condition-action pairs specifying protocol-mandated responses to positive pregnancy tests, and partner requirement specifications applicable to partners of enrolled subjects.

With continued reference to FIG. 23, the method 2300 proceeds to a step 2308 of human validation of the extracted data and logic. At step 2308, the human validation interface 2106 presents the structured data fields extracted At step 2304 and the conditional logic extracted At step 2306 to a qualified human reviewer for verification against the source protocol document. The step 2308 implements the validation gate that prevents AI hallucinations from propagating downstream into the deterministic generation zone. The human reviewer At step 2308 can include a clinical research coordinator, clinical operations specialist, or medical monitor who confirms that the AI-extracted data accurately represents the source natural language protocol text.

According to some embodiments, the method 2300 includes a decision 2310 that determines whether the extraction is approved. At the decision 2310, the human validator evaluates the accuracy and completeness of the AI-extracted structured data and conditional logic. If the extraction is not approved at the decision 2310, the method 2300 loops back to the step 2304 for correction, enabling iterative refinement of the AI-extracted data until the human validator confirms accuracy. The loop between the decision 2310 and the step 2304 ensures that only validated data proceeds to subsequent steps in the method 2300.

According to another embodiment, when the extraction is approved at the decision 2310, the method 2300 continues to a step 2312 of storing validated data in the source of truth. At step 2312, the storage component stores the validated structured protocol data object in the validated source of truth 2108 in a locked state. The step 2312 establishes the architectural boundary between the AI-permitted zone and the AI-prohibited zone, ensuring that all subsequent processing in the method 2300 operates on validated data without any AI inference. The validated data stored At step 2312 includes the structured data fields, the conditional logic, the three-layer decomposition of interventional entities, the concomitant therapy rule arrays, the discontinuation criteria condition trees, the follow-up rule specifications, and the reproductive safety requirement objects extracted and validated through the preceding steps.

According to further embodiments, the method 2300 proceeds to a step 2314 of selecting a target document template. At step 2314, the system receives a selection indicating which document template from the document templates 2112 should be generated. The target document template selected At step 2314 can include treatment checklists, grids, patient calendars, informed consent form drafts, EDC templates, pharmacy dispensation schedules, regulatory submission templates, or eligibility criteria mapped to EMR/EHR structured query fields. According to one example, the step 2314 can receive a selection for a treatment checklist template that will be populated with procedure schedules, dosing instructions, and safety monitoring requirements from the validated source of truth 2108.

According to some embodiments, the method 2300 continues to a step 2316 where a template hydration engine invokes smart fields. At step 2316, the document template selected At step 2314 encounters smart field placeholders within the template structure and invokes the smart field intermediary 2110 to resolve data for each placeholder. The template hydration engine At step 2316 processes the selected template and identifies each smart field that requires data from the validated source of truth 2108. The smart fields invoked At step 2316 implement the universal smart field 2000 architecture including the data binding interface 2002, the context resolution logic 2004, the categorization metadata 2006, and the template output adapter 2008.

According to another embodiment, the method 2300 proceeds to a step 2318 where smart fields resolve data from the intermediary model. At step 2318, each smart field invoked At step 2316 queries the context resolution module 2208 with contextual metadata including document type, study type, and user role. The context resolution module 2208 determines the appropriate data representation based on the contextual factors, and the universal intermediary 2210 retrieves resolved data from the validated source of truth 2212. The step 2318 retrieves data including procedure-based associations, vocabulary identifiers from multiple clinical standards, protocolRules references, and protocol-specific operational parameters stored in the validated source of truth 2108. According to one example, the step 2318 can resolve an intervention smart field by retrieving definition layer data, logistics layer data, and task layer data with explicit referential links between layers from the validated source of truth 2108.

The context resolution module 2208 operates as a deterministic lookup function with the following signature: resolve (procedure_node, context_metadata)→endered_data_representation, where context metadata is a structured object containing: (A) document_type: the type of downstream artifact being generated (e.g., “patient_calendar”, “EDC_form”, “treatment_checklist”, “billing_grid”, “ICF”, “pharmacy_manual”); (B) study_type: The protocol phase and therapeutic area (e.g., “Phase_II_oncology”, “Phase_I_dose_escalation”); and (C) user_role: The intended consumer of the document (e.g., “patient”, “site_coordinator”, “data_manager”, “pharmacist”, “financial_coordinator”). The module executes a two-level lookup: (1) Document type×service type→selects the rendering template and the set of CPTM fields to extract (2) User role→selects the language register and level of technical detail for the rendered output. The resolution is a static rule table with no AI inference.

Example—the same CMP laboratory service node rendered for three different contexts: Context A: {document_type: “patient_calendar”, user_role: “patient”} The module selects the patient-facing rendering template for laboratory services. Output: “Blood draw (fasting required—no food or drink after midnight the night before). You will receive a call from the scheduling coordinator 48 hours in advance. Plan to arrive 30 minutes early. Estimated time at site: 45 minutes.” Fields consumed from the procedure node: fastingRequired, collection Timing, schedule[ ]. windowMinus/windowPlus, visits[ ].estimatedDuration. Context B: {document_type: “EDC_form”, user_role: “data_manager”} The module selects the EDC field configuration template for laboratory services. Output: CRF form with fields: LBTEST (“Alanine Aminotransferase”), LBSTRESU (“IU/L”), LBDTC (date/time of collection), LBBLFL (baseline flag), edit check: if LBSTRESN>3×LBNRHI then flag Grade 2 (action: monitor); if LBSTRESN>5×LBNRHI then flag Grade 3 (action: hold_treatment). Fields consumed from the procedure node: terminology.loincCode, terminology.cdiscDomain, tests[ ].name, tests[ ].units, ruleLinks[ ]→protocolRules.safety Thresholds[ ]. Context C: {document_type: “billing_grid”, user_role: “financial_coordinator”}

The module selects the line item template for laboratory services. Output: Line item: “Comprehensive Metabolic Panel|CPT 80053|Visit: C1D1|Arm: All Arms|Billable To: Sponsor|Classification: Research|Rate: [FMV lookup]” Fields consumed from the procedure node: type, fields.panelType, schedule[ ].visitId, schedule[ ].armId, fields.billableTo.value. In the examples, the same locked CPTM procedure node is the source. The context resolution module determines which fields to read and how to render them—entirely through the static lookup table, with no model inference

With continued reference to FIG. 23, the method 2300 continues to a step 2320 where the document template is populated with resolved data. At step 2320, the template output adapter 2008 formats the resolved data from the step 2318 for insertion into the template fields. The step 2320 populates each smart field placeholder in the selected template with context-appropriate content derived from the validated source of truth 2108 through deterministic field mapping without any AI inference. The populated template At step 2320 includes all procedure schedules, dosing parameters, conditional logic representations, safety thresholds, and other protocol-specific data required for the selected document type.

Example locked.cptm fields: interventions[0]: name: “MK-8527 11 mg Tablet”; genericName: “islatravir”; productType.value: “Drug”; treatmentRole.value: “Experimental”; formulation.value: “Tablet”; strength.value: 11, strength.units: “mg”; route.value: “Oral”; dose.value: 11, dose.units: “mg”; frequency: “Once weekly”; blindingStatus. value: “Double-blind”;
ipHandling[0]: storageRequirements: “15-30° C., protect from moisture”; accountabilityMethod: “Drug accountability log+pill count at each visit”; blindingMaintenance: “Over-encapsulated; identical appearance for active and placebo”; services[administration-001]; fields.firstDose WitnessedRqd: true; fields.firstDose VisitId: “v-cld1”; fields.complianceAssessment: “Pill count and dosing diary”; fields. fastingRequired: false; fields.preAdministrationChecks: “Confirm ANC >500/mm³; confirm labs within 72 h of visit”; fields.dispensingQuantity: “4-week supply”; fields.pillCountRequired: true; fields.return Verification: true; fields.administrationWindow.standard.value: 3; fields.administration Window.standard.units: “days” 1; fields.administrationWindow.standard.direction: “both”—can be updated using deterministic filed-path substitution as shown in Table V.

TABLE V

| Output Field | Mapping Rule | Resolved Value |
|---|---|---|
| Study Drug Name | interventions[0].name | “MO-324 11 mg Tablet” |
| Generic Name | interventions[0].genericName | “islatravir” |
| Dose | interventions[0].dose.value · “ ” + dose.units | “11 mg” |
| Formulation | interventions[0].formulation.value | “Tablet” |
| Route | interventions[0].route.value | “Oral” |
| Frequency | interventions[0].frequency | “Once weekly” |
| Blinding | interventions[0].blindingStatus.value | “Double-blind” |
| Fasting Required | services[adm].fields.fastingRequired → bool → | “No” |
| | “Yes”/“No” | |
| First Dose Witnessed | services[adm].fields.firstDoseWitnessedRqd → | “Yes - at Visit C1D1” |
| | bool | |
| Pre-dose Checks | services[adm].fields.preAdministrationChecks | “Confirm ANC >500/mm³; confirm |
| | | labs within 72 h of visit” |
| Administration | administrationWindow.standard.value · units · | “±3 days” |
| Window | direction | |
| Dispensing Quantity | services[adm].fields.dispensingQuantity | “4-week supply” |
| Pill Count Required | services[adm].fields.pillCountRequired → bool | “Yes” |
| Storage | ipHandling[0].storageRequirements | “15-30° C., protect from moisture” |
| Drug Accountability | ipHandling[0].accountabilityMethod | “Drug accountability log + pill count |
| | | at each visit” |
| Return Required | services[adm].fields.returnVerification → bool | “Yes - return all unused/empty |
| | | packages” |
| Compliance Tracking | services[adm].fields.complianceAssessment | “Pill count and dosing diary” |

The example table defines a direct field-path lookup. The template adapter at Step 2320 iterates through the template's content control identifiers, finds the corresponding CPTM field path in the hardcoded mapping table, reads the value from the locked CPTM object, and inserts it.

According to some embodiments, the method 2300 proceeds to a step 2322 where the final protocol-specific document is generated. At step 2322, the system produces the completed operational document with all smart field placeholders populated with validated data. The protocol-specific document generated At step 2322 maintains consistency with the validated source of truth 2108 and reflects the specific requirements of the clinical trial protocol received At step 2302. According to one example, the step 2322 can generate a treatment checklist that includes procedure schedules derived from the task layer data, dosing instructions derived from the definition layer data, and safety monitoring thresholds derived from the protocolRules section of the validated source of truth 2108.

According to another embodiment, the method 2300 concludes with a step 2324 where the document is encrypted and exported in the proprietary .cptm format for secure distribution. At step 2324, the encrypted export 2114 applies encryption to the document content generated At step 2322 using an encryption algorithm. The step 2324 converts the encrypted document into the proprietary .cptm file format and transmits the encrypted .cptm output through a transit security layer to authorized recipients. The step 2324 ensures that the generated protocol-specific document is fully encrypted and inaccessible without proper authorization both at rest and in transit. According to one example, the step 2324 can encrypt a generated pharmacy dispensation schedule, convert the encrypted document to the .cptm format, and distribute the encrypted .cptm output to authorized clinical trial sites while blocking unauthorized access attempts from accessing the encrypted content.

According to further embodiments, the system achieves data compression by transforming 200-400 page narrative documents into structured data objects orders of magnitude smaller through a cross-reference architecture where procedures link to associated data elements via reference identifiers rather than embedded copies. The generated document processed by the system derives from the compressed structured data format that links procedure-based information to associated clinical trial data through reference identifiers. The cross-reference architecture reduces the data footprint of the encrypted .cptm output compared to conventional approaches that embed complete copies of referenced data within each document. According to one example, a treatment checklist generated as the generated document references procedure definitions, dosing parameters, and safety thresholds through identifiers that resolve to shared definitions in the validated source of truth, rather than embedding complete copies of each referenced element within the treatment checklist document.

According to another embodiment, the system operates in conjunction with three independently incrementing version dimensions maintained by the system: a content version for substantive protocol changes, a schema version for underlying data model version, and an amendment version for regulatory amendment numbering. The generated document processed by the system carries version information indicating the content version, schema version, and amendment version of the source protocol data from which the document was generated. The three-dimensional versioning enables compliance evaluation against the specific protocol state in effect at the time of a clinical event. An encrypted .cptm output preserves the version information from the generated document, enabling the authorized recipient to identify which protocol version the document reflects. According to one example, the encrypted .cptm output can include version metadata indicating that the generated document was produced from content version 3, schema version 2.1, and amendment version 2 of the clinical trial protocol, enabling the authorized recipient to verify that the document reflects the current protocol state or identify when the document was generated from a superseded protocol version.

According to various embodiments, the system implements a mapping diagram 2500 that illustrates the transformation between natural language protocol elements 2502 and structured data fields 2504. Referring to FIG. 25, the mapping diagram 2500 demonstrates how diverse natural language protocol content is systematically converted into structured, machine-readable data fields suitable for consumption by the smart field intermediary 2110. The mapping diagram 2500 provides a visual representation of the transformation process that enables the system to accept unstructured or multi-format clinical trial data and output the compressed data format including respective procedure, procedure-based associations, and associated clinical trial data.

According to one embodiment, the natural language protocol elements 2502 include dosing instructions expressed in natural language form within clinical trial protocol documents. As shown in FIG. 25, a dosing instruction such as “Administer 20 mg oral dose daily” maps to a Numeric Dose Field within the structured data fields 2504. The Numeric Dose Field contains structured parameters, including value, unit, route, and frequency, that encode the dosing instruction in a machine-readable format. The mapping from the natural language dosing instruction to the Numeric Dose Field extracts the numeric value of 20, the unit of mg, the route of oral administration, and the frequency of daily dosing, storing each parameter in a corresponding structured field. According to one example, the AI processing engine 2104 parses the natural language dosing instruction to identify each parameter component and maps the identified components to the structured parameters within the Numeric Dose Field.

According to some embodiments, the natural language protocol elements 2502 include conditional statements that express threshold comparisons and resulting actions. As shown in FIG. 25, a conditional statement such as “If blood level exceeds 5.0, stop treatment” maps to a Conditional Logic Field within the structured data fields 2504. The Conditional Logic Field encodes the threshold comparison and resulting action as structured IF/THEN operators. The mapping from the natural language conditional statement to the Conditional Logic Field extracts the condition parameter of blood level, the comparison operator of exceeds, the threshold value of 5.0, and the action of stop treatment. The Conditional Logic Field stores these elements as structured logical operators that downstream systems can evaluate computationally against patient clinical data without manual interpretation. According to one example, the method 1900 for converting natural language conditional statements into executable machine-readable logic modules processes the conditional statement to generate the Conditional Logic Field with IF/THEN operators encoding the threshold comparison and action trigger.

According to another embodiment, the natural language protocol elements 2502 include device specifications that identify intervention types and device identifiers. As shown in FIG. 25, a device specification such as “Use infusion pump device Model X” maps to an Intervention Type Field within the structured data fields 2504. The Intervention Type Field categorizes the intervention type and stores the device name in structured format. The mapping from the natural language device specification to the Intervention Type Field extracts the intervention category of device, the device type of infusion pump, and the device identifier of Model X. The Intervention Type Field enables the universal smart field 2000 to resolve intervention data appropriately based on the categorization metadata 2006 that stores classification information indicating whether bound data represents a medical device intervention, an infusion intervention, a surgical intervention, or an oral medication intervention.

With continued reference to FIG. 25, the natural language protocol elements 2502 include scheduling instructions that specify visit timing within clinical trial protocols. A scheduling instruction such as “Treatment visits on days 1, 7, 14, 28” maps to a Timing Schedule Field within the structured data fields 2504. The Timing Schedule Field encodes the visit schedule as a structured array containing the individual day values. The mapping from the natural language scheduling instruction to the Timing Schedule Field extracts the visit type of treatment visits and the timing values of days 1, 7, 14, and 28, storing the timing values as an ordered array within the structured field. The Timing Schedule Field enables the system to generate patient calendars with window calculations and treatment checklists that reflect the scheduled visit timing derived from the natural language protocol text.

According to some embodiments, the mapping illustrated in the mapping diagram 2500 enables the smart field intermediary 2110 to consume diverse natural language protocol content through a standardized transformation process. The data binding interface 2002 of the universal smart field 2000 receives input data that has been transformed from the natural language protocol elements 2502 into the structured data fields 2504 through the mapping process. The context resolution logic 2004 can then determine the appropriate data representation based on contextual metadata because the underlying data has been normalized into structured fields with consistent parameter formats. According to one example, when the smart field intermediary 2110 receives a request for intervention data, the smart field intermediary 2110 can resolve the request against Intervention Type Fields that have been populated through the mapping process, enabling context-appropriate output regardless of whether the original natural language protocol text described a medical device, an infusion, a surgical procedure, or an oral medication.

According to further embodiments, the system implements a LOINC enrichment pipeline that maps laboratory test descriptions from the natural language protocol elements 2502 to standardized vocabulary identifiers within the structured data fields 2504. The LOINC enrichment pipeline comprises AI normalization that outputs a canonical name and a confidence signal from raw service names extracted from the natural language protocol text. The AI normalization step transforms free-text procedure descriptions into standardized intermediate representations suitable for vocabulary lookup. Following the AI normalization step, the LOINC enrichment pipeline performs deterministic database lookup against the LOINC vocabulary to identify candidate codes matching the normalized canonical name. The deterministic database lookup queries a local LOINC database and returns a candidate code along with a match confidence score.

According to another embodiment, the LOINC enrichment pipeline includes a confidence gate that writes vocabulary identifiers to the structured data fields 2504 only if the confidence score meets a predefined threshold. Below-threshold results are flagged for human review rather than being automatically written to the structured data format. The confidence gate prevents low-confidence vocabulary assignments from entering the validated source of truth 2108 without human verification. According to one example, when the AI normalization step produces a canonical name with a confidence signal below the threshold, the system flags the vocabulary assignment for review through the human validation interface 2106 rather than writing an uncertain LOINC code to the structured data fields 2504.

According to some embodiments, the LLM within the AI processing engine 2104 is architecturally prohibited from generating LOINC codes directly. The LLM produces only normalized names that feed the deterministic database queries within the LOINC enrichment pipeline. This architectural constraint prevents hallucinated vocabulary codes from entering clinical data systems by ensuring that all vocabulary code assignments result from deterministic lookup operations rather than LLM inference. The LLM cannot output a LOINC code as part of the AI extraction process; instead, the LLM outputs normalized canonical names that serve as input to the deterministic database lookup step. According to one example, when processing a laboratory procedure description from the natural language protocol elements 2502, the LLM outputs a normalized name such as “serum creatinine” along with disambiguation context, and the deterministic database lookup queries the LOINC vocabulary to identify the corresponding LOINC code for serum creatinine measurement, preventing the LLM from generating a plausible-looking but incorrect LOINC code through hallucination.

According to further embodiments, the system maps eligibility evaluation service objects to external patient record standards for automated pre-screening. Eligibility evaluation service objects extracted from the natural language protocol elements 2502 and stored within the structured data fields 2504 are mapped via deterministic lookup tables to OMOP CDM and HL7 FHIR patient record fields. The deterministic lookup tables establish correspondences between eligibility criteria parameters in the structured data format and standardized patient record fields in the OMOP Common Data Model and HL7 FHIR specifications. The mapping generates structured database queries that can be executed against a health system's electronic medical record to identify potentially eligible patients. The structured database queries are generated through deterministic field mapping without LLM involvement at the point of use where the queries touch patient data. According to one example, an eligibility criterion specifying “age between 18 and 65 years” extracted from the natural language protocol elements 2502 is mapped via the deterministic lookup table to the corresponding OMOP CDM person table birth\_datetime field or HL7 FHIR Patient resource birthDate element, generating a structured query that can identify patients within the specified age range without any AI inference at the query generation or execution step.

According to various embodiments, the system implements a protocolRules section within the structured data format that provides machine-readable representation of protocol operational logic. The protocolRules section encodes clinical trial safety logic, monitoring criteria, medication restrictions, discontinuation conditions, follow-up obligations, and reproductive safety requirements as structured data objects that downstream systems can evaluate computationally without manual interpretation of narrative protocol text.

According to one embodiment, the protocolRules section includes safety threshold objects that encode graded severity monitoring criteria for clinical parameters. Each safety threshold object contains a parameter identifier conforming to an external vocabulary standard such as a LOINC code that identifies the specific clinical measurement. Each safety threshold object contains a comparator field specifying the comparison operation such as greater than, less than, equals, or between. Each safety threshold object contains a value field with a numeric threshold and a unit field specifying the measurement unit. Each safety threshold object contains an action trigger field specifying the response to be taken when the threshold is exceeded, where the action trigger can include hold\_treatment, notify\_PI, dosel_reduce, or discontinue designations. Each safety threshold object contains a severity grade field with a numeric grade from 1 to 4 per CTCAE or protocol-specific grading scales. According to one example, an absolute neutrophil count parameter can be associated with multiple safety threshold objects creating a graded severity ladder: a Grade 2 threshold object specifying ANC less than 1.5×10⁹/L with action trigger of monitor, a Grade 3 threshold object specifying ANC less than 1.0×10⁹/L with action trigger of hold\_treatment, and a Grade 4 threshold object specifying ANC less than 0.5×10⁹/L with action trigger of discontinue.

According to some embodiments, the protocolRules section includes concomitant therapy rule arrays that encode medication class restrictions with classification designations and temporal parameters. Each concomitant therapy rule object contains a medication class field identifying the medication or therapy class by ATC code or class name. Each concomitant therapy rule object contains a status field with an enumerated value specifying the restriction classification, where the status can include allowed, prohibited, restricted, or requires\_washout designations. Each concomitant therapy rule object contains a washout period field with a duration object specifying the required washout time before or after the restricted medication, where the duration object includes a value and a unit. Each concomitant therapy rule object contains a conditions array specifying when the restriction applies based on clinical circumstances. Each concomitant therapy rule object contains an exceptions array specifying conditions under which the restriction does not apply. The concomitant therapy rule arrays enable downstream systems to evaluate medication compatibility computationally against the encoded restrictions without manual cross-referencing of narrative protocol text.

According to another embodiment, the protocolRules section includes discontinuation criteria encoded as evaluatable condition trees. Each discontinuation criterion is represented as a Boolean logic tree data structure with nodes containing operator fields for AND, OR, and NOT operations. The nodes contain parameter reference fields identifying clinical measurements by LOINC code or other vocabulary identifiers. The nodes contain comparator fields specifying comparison operations and threshold fields with numeric values and units. The nodes can include child node arrays enabling arbitrary nesting depth to represent complex multi-criteria discontinuation conditions. Each discontinuation criterion object contains a criterion type field with an enumeration specifying the discontinuation category, where the criterion type can include investigator\_decision, protocol\_specified, sponsor\_directed, or patient\_withdrawal classifications. Each discontinuation criterion object contains an action required array specifying the procedural steps that must be completed upon discontinuation. Each discontinuation criterion object contains a follow-up plan reference linking to a follow-up rule object that defines post-discontinuation visit requirements.

According to further embodiments, the protocolRules section includes follow-up rule objects that specify post-event clinical obligations as structured data. Each follow-up rule object contains a trigger event field referencing a clinical event type that initiates the follow-up obligation, where the trigger event can include serious adverse event “SAE,” treatment\_discontinuation, or study\_completion designations. Each follow-up rule object contains a follow-up type field specifying the category of follow-up, where the follow-up type can include safety\_follow\_up, survival\_follow\_up, or long\_term\_follow\_up classifications. Each follow-up rule object contains a schedule windows array with visit window objects specifying timing relative to the trigger event. Each follow-up rule object contains a required services array with identifiers specifying which clinical procedures must be performed at each follow-up visit. Each follow-up rule object contains a termination condition field with a condition tree specifying when follow-up ends. According to one example, a follow-up rule object can specify that safety follow-up is triggered by treatment discontinuation, with schedule windows at 7 days, 14 days, and 30 days after the trigger event, required services including vital signs and laboratory assessments, and a termination condition specifying that follow-up ends when all adverse events have resolved to Grade 1 or baseline.

According to some embodiments, the protocolRules section includes reproductive safety requirement objects that encode sex-specific contraception mandates, pregnancy testing schedules, and action-on-positive logic. Each reproductive safety requirement object contains an affected sex field specifying whether the requirement applies to male subjects, female subjects, or both. Each reproductive safety requirement object contains a contraception requirements array with contraception method objects specifying method type, duration, and start timing relative to treatment. Each reproductive safety requirement object contains a pregnancy test schedule object specifying test type as serum or urine hCG, frequency of testing, and timing relative to dosing. Each reproductive safety requirement object contains an action-on-positive field with a condition-action pair specifying the protocol-mandated response to a positive pregnancy test. Each reproductive safety requirement object contains a partner requirements field specifying contraception or testing requirements applicable to partners of enrolled subjects.

According to another embodiment, the system implements a per-patient instance of the structured data format that hydrates each protocol-defined procedure node with patient-specific data. The per-patient instance simultaneously holds expected data from the locked structured protocol data object, actual recorded event data from the patient's clinical encounters, and computed deviation fields evaluated against the protocolRules logic trees. The expected data includes protocol-defined schedule windows, parameters, and rule thresholds from the validated source of truth. The actual data includes the patient's real recorded event specifying what happened, when the event occurred, and what was measured. The deviation field represents a computed comparison between expected and actual values evaluated against the protocolRules logic trees. The per-patient instance enables individual patient journey visualization in protocol terms, cross-patient comparison across enrollees in the same study, automated deviation detection as a first-class operation, population-level identification of which procedures are systematically generating deviations, and regulatory deviation reporting with structured auditable evidence.

According to some embodiments, the system generates clinical trial study budgets by extracting procedure service records from the structured data format. Each procedure service record contains procedure type, visit frequency, arm assignment, and handling logistics parameters. The system maps each procedure service record to a cost basis through deterministic rate tables that associate procedure types with fair market value rates. The system generates a structured budget object with line items referencing the originating procedure node by identifier. The referential linkage between budget line items and procedure nodes enables protocol amendments that modify a procedure node to propagate to the budget line items referencing that procedure. According to one example, when a protocol amendment changes a PK blood draw from 4 timepoints to 6 timepoints at a specific visit, the budget line item referencing that procedure node automatically reflects the updated visit frequency without manual budget reconstruction.

According to further embodiments, the system implements a two-tier protocol object architecture with a canonical protocol layer and a site instance layer. The canonical protocol layer holds sponsor-defined parameters including procedure definitions, schedule windows, and rule thresholds. The canonical protocol layer remains immutable after lock and auditable against the sponsor's original protocol. The site instance layer for each participating site holds site-local overrides including local lab reference ranges, local IRB identifiers, local vendor contacts, and local form numbering. The site instance layer references canonical nodes without modifying canonical field values. The canonical layer and each site instance layer are independently versioned. Site-specific operational documents are generated by combining the canonical and site-instance layers through deterministic template instantiation. The two-tier architecture enables the canonical layer to remain independently auditable while accommodating site-local configuration requirements.

According to another embodiment, the system generates Interactive Response Technology configurations from structured objects within the validated source of truth. The system extracts randomization scheme parameters including randomization method, block size, and allocation ratio from structured randomization service objects. The system extracts stratification factors specifying the clinical characteristics used to stratify randomization. The system extracts arm definitions specifying the treatment arms and their associated intervention parameters. The system extracts drug supply visit schedules specifying when investigational product is dispensed to subjects. The system extracts dispensing rules specifying the quantity and packaging of investigational product per dispensation. The system generates a machine-readable IRT configuration file by deterministic mapping of the extracted parameters to an IRT system schema without AI inference at the configuration generation step. The deterministic generation of IRT configurations from structured protocol objects eliminates the manual configuration process that conventionally requires specialist consultants to read protocol documents and manually configure IRT systems.

According to various embodiments, the system derives site capability profiles by aggregating validated structured protocol data objects from prior studies conducted at each clinical trial site. The aggregation process extracts procedure types, service categories, therapeutic areas, study phases, and complexity metrics from the validated structured protocol data objects associated with studies previously conducted at a site. The site capability profiles provide empirical capability data derived from validated protocol data objects representing actual conducted studies rather than self-reported site questionnaires or external data sources. According to one embodiment, the system aggregates procedure type coverage from the validated structured protocol data objects associated with each site. The procedure types extracted during aggregation correspond to the 14 defined service types in the service type taxonomy including administration, assessment, ECG, vital signs, laboratory, imaging, biospecimen, informed consent, questionnaire, procedure, concomitant medication, safety monitoring, randomization, and eligibility evaluation. The aggregation process counts the number of studies at each site that included each procedure type, generating procedure type coverage vectors that indicate which procedure categories the site has demonstrated experience executing. According to one example, the aggregation process can determine that a site has conducted 15 studies including laboratory services, 12 studies including imaging services, and 3 studies including biospecimen services, generating a procedure type coverage vector reflecting the site's demonstrated experience across these service categories.

According to some embodiments, the system extracts service categories from the validated structured protocol data objects to populate the site capability profiles. The service categories provide finer granularity than procedure types by distinguishing between different clinical activities within each procedure type. The aggregation process identifies the specific service categories represented in each validated structured protocol data object and accumulates service category counts across all studies associated with each site. According to one example, within the laboratory procedure type, the aggregation process can distinguish between hematology services, chemistry services, urinalysis services, and specialized biomarker services, generating service category coverage data indicating which specific laboratory service categories the site has demonstrated experience performing.

According to another embodiment, the system extracts therapeutic area coverage from the validated structured protocol data objects associated with each site. The therapeutic area extraction identifies the disease categories and indication types represented in each validated structured protocol data object. The aggregation process accumulates therapeutic area counts across all studies associated with each site, generating therapeutic area coverage vectors that indicate which disease categories the site has demonstrated experience treating within clinical trial contexts. According to one example, the aggregation process can determine that a site has conducted 8 oncology studies, 5 cardiovascular studies, and 2 infectious disease studies, generating a therapeutic area coverage vector reflecting the site's demonstrated experience across these therapeutic categories.

According to further embodiments, the system extracts study phase information from the validated structured protocol data objects to populate the site capability profiles. The study phase extraction identifies whether each validated structured protocol data object represents a Phase I, Phase II, Phase III, or Phase IV clinical trial. The aggregation process accumulates study phase counts across all studies associated with each site, generating study phase coverage vectors that indicate which trial phases the site has demonstrated experience conducting. According to one example, the aggregation process can determine that a site has conducted 2 Phase I studies, 6 Phase II studies, and 4 Phase III studies, generating a study phase coverage vector reflecting the site's demonstrated experience across these trial phases.

According to some embodiments, the system extracts complexity metrics from the validated structured protocol data objects to populate the site capability profiles. The complexity metrics include protocolRules logic tree density, PK timepoint counts, and biospecimen type diversity. The protocolRules logic tree density measures the number of nodes within the Boolean logic tree data structures encoding AESI definitions, safety threshold objects, discontinuation criteria condition trees, and other conditional logic within the protocolRules section of each validated structured protocol data object. The PK timepoint counts measure the number of pharmacokinetic sampling timepoints specified within each validated structured protocol data object. The biospecimen type diversity measures the number of distinct biospecimen types collected within each validated structured protocol data object. The aggregation process accumulates complexity metric values across all studies associated with each site, generating complexity metric profiles that indicate the operational complexity levels the site has demonstrated experience handling.

According to another embodiment, biospecimen service objects within the validated structured protocol data objects represent scheduled collections where specimen collection is the primary clinical act. The biospecimen service objects carry service type schemas, visit linkage, and handling logistics parameters for scheduled specimen collection activities. Tissue from procedures is carried as fields on the originating procedure service object rather than as independent biospecimen service objects. Both biospecimen service objects and tissue fields on procedure service objects feed a unified sample handling logistics section at the root level of the structured data format. The aggregation process extracts biospecimen type diversity by counting the distinct biospecimen types represented across both biospecimen service objects and tissue fields on procedure service objects within each validated structured protocol data object associated with a site.

According to some embodiments, the system performs automated site feasibility matching by comparing a new protocol's procedure requirements against site capability profiles. The automated site feasibility matching receives a new validated structured protocol data object representing a clinical trial for which site selection is required. The automated site feasibility matching extracts the procedure requirements from the new validated structured protocol data object, including procedure types, service categories, therapeutic area, study phase, and complexity metrics. The automated site feasibility matching compares the extracted procedure requirements against each candidate site's capability profile through deterministic field comparison without AI inference at the matching step.

According to further embodiments, the automated site feasibility matching generates quantitative feasibility scores for each candidate site. The quantitative feasibility scores reflect the degree of match between the new protocol's procedure requirements and each site's demonstrated capability profile. The feasibility score calculation compares each procedure type required by the new protocol against the procedure type coverage vector in the site capability profile. The feasibility score calculation compares each service category required by the new protocol against the service category coverage data in the site capability profile. The feasibility score calculation compares the therapeutic area of the new protocol against the therapeutic area coverage vector in the site capability profile. The feasibility score calculation compares the study phase of the new protocol against the study phase coverage vector in the site capability profile. The feasibility score calculation compares the complexity metrics of the new protocol against the complexity metric profiles in the site capability profile. The quantitative feasibility score aggregates the comparison results across all requirement dimensions to produce a single numeric score indicating overall site feasibility for the new protocol.

According to another embodiment, the automated site feasibility matching generates capability gap reports identifying procedures the new protocol requires that the site has no demonstrated history with. The capability gap reports compare each procedure type, service category, and complexity metric required by the new protocol against the corresponding coverage data in the site capability profile. When the comparison identifies a procedure requirement for which the site capability profile contains no demonstrated experience, the capability gap report records the identified gap. The capability gap reports enable sponsors to identify which specific capabilities a site lacks relative to the new protocol requirements, supporting informed site selection decisions and targeted site training or support planning. According to some embodiments, the automated site feasibility matching generates site comparison rankings across enrolled sites in a network. The site comparison rankings order candidate sites by quantitative feasibility score, enabling sponsors to identify which sites in the network have the strongest demonstrated capability match for the new protocol. The site comparison rankings can be filtered by geographic region, therapeutic area experience, or other selection criteria to support site selection workflows. The site comparison rankings operate on structured data elements derived from validated protocol data objects that are not available in external databases or self-reported questionnaires.

According to further embodiments, the system generates EHR clinical note scaffolds with pre-populated fields for required procedures at specified visits. The EHR clinical note scaffolds extract the procedures, assessments, and measurements required at each visit from the validated structured protocol data object. The EHR clinical note scaffolds include pre-populated fields for each required procedure with expected normal ranges derived from protocol-defined threshold objects within the protocolRules section. The EHR clinical note scaffolds include referential links back to the originating protocol procedure nodes, enabling each pre-populated field to carry a machine-readable reference to the protocol procedure that required the clinical activity. According to one example, an EHR clinical note scaffold for a treatment visit can include pre-populated fields for vital signs with expected normal ranges derived from safety threshold objects, laboratory assessments with expected normal ranges derived from safety threshold objects, and procedure completion indicators, where each field carries a referential link to the corresponding service object within the validated structured protocol data object.

According to another embodiment, the system derives trial master file document taxonomy by mapping protocol activities to TMF document categories through deterministic lookup. The trial master file document taxonomy derivation extracts the set of regulatory, ethical, clinical, and operational activities from the validated structured protocol data object. The derivation process maps each extracted activity to TMF document categories through a deterministic lookup table that associates activity types with corresponding TMF Reference Model document categories. The derivation process generates a study-specific document index with required document types derived from the protocol activities. The study-specific document index includes expected submission timing for each required document type based on the protocol schedule and regulatory requirements. The trial master file document taxonomy is generated prior to collection of any study documents, enabling proactive document management planning based on the specific activities defined in the validated structured protocol data object. According to one example, when the validated structured protocol data object includes informed consent service objects, the deterministic lookup maps the informed consent activities to TMF document categories for consent form templates, IRB approval documentation, and signed consent form storage, generating document index entries with expected submission timing aligned to the enrollment schedule defined in the protocol.

### IMPLEMENTATION EXAMPLES

The following implementation examples provides description of functionality of various embodiments of the system, method, processes, and/or functionality that can be implemented alone or in conjunction with other examples and embodiments herein, and each combination is explicitly described by inclusion in this section.

### Constrained AI Data

According to various embodiments, the system implements a state machine with a plurality of states: uploaded→ai processing→ai review→human review→pending_signoff→approved→failed. The system also implements segregated data layers or operational zones in which AI can be used to capture, ingest, and/or process data, and constrained layers or zone in which AI is not permitted to prevent infiltration of unvalidated data This separation provide data integrity and assurance unavailable in conventional solutions. In one example, the AI-permitted zone spans ai processing and ai_review. The human validation gate is human_review through pending_signoff. The transition to “approved” (locked state) is an example of the architectural boundary. Once a cptm object reaches “approved,” it enters the AI-prohibited zone. Examples of what technically prevents AI from infiltrating the AI-restricted layer: generation pipeline (everything after a CPTM is locked/approved) is implemented as a completely separate code path with no dependency on any AI service: the template rendering functionality reads from the validated cptm data object and performs deterministic field substitution via specific function calls (e.g., renderWithReplacements( )). This function does string interpolation against CPTM fields. In various examples, it imports no AI libraries, makes no API calls to any LLM endpoint, and contains no probabilistic logic. In some examples, the system can be configured to confirm checksum or other validation data produced for and/or stored with locked data objects. In further embodiment, the system implements a smart field intermediary layer configured to resolve fields from the locked CPTM through a context resolution module that uses deterministic lookup, and for example, not inference. Resolution is based on document type, study type, and user role metadata, all evaluated through conditional logic (e.g., procedural coding).

In further embodiments, the system enforces edit permissions through role-based access control. The save function writes to a versioned endpoint. The locked state is enforced at both the application layer (UI prevents mutation of approved CPTMs) and the API layer (backend rejects write operations on locked records). In some examples, modification of locked data is permitted via propagation functions discussed herein, however, the system can enforce/prohibit modifications that are not validated as discussed with respect to achieving the locked status. In some embodiments, there is no code path, import, or API call that would allow any LLM or neural network to contribute to the output of Tier 2. The separation is not a policy or convention; but rather a structural property of the codebase, that has no corollary in conventional systems. The segregation (e.g., Tier 2 modules) physically prevents calls to AI services, and likewise such dependencies do not exist in their import chain.

In some examples, once a data generation job or function reaches “approved” status (e.g., via the signOff endpoint), a snapshot (e.g., of the cptm) is stored as a JSONB column. The application layer checks job status before permitting any mutation. The pushToStudy endpoint transfers the approved CPTM to the study record. At that point, the study's CPTM is the validated, locked artifact from which all documents are generated. The version history (listVersions, getVersion, compare Versions in the same service file) maintains an append-only audit trail. In further example, restoring a previous version creates a new version entry; it does not overwrite history.

In some embodiments, the “lock” is achieved through: (1) a database-level status field (e.g., on the ConversionJob record) set to “approved” via an authenticated API endpoint requiring superadmin or assigned-reviewer authorization; (2) application-layer enforcement where all mutation endpoints check job status before permitting changes; (3) version snapshots stored as immutable JSONB records with actor identity, timestamp, change type, and full CPTM snapshot; (4) the pushToStudy endpoint that transfers the approved snapshot to the study's CPTM record, creating the production artifact from which all Tier 2 generation operates.

Executable Code Example Integrated into Data Object

In various embodiments, executable code can be integrated/generated as part of the data object creation. Examples of executable code included in the CPTM data object include any or more or any combination of: (a) Boolean logic trees for safety evaluation. AESI definitions encoded as nested AND/OR/NOT operator nodes with clinical parameter references (LOINC codes), comparators (greater_than, less_than, between), and threshold values with units. Example: Hy's Law criteria encoded as an OR node containing a threshold node (ALT >5×ULN) and an AND node containing two child thresholds (ALT >3×ULN AND total bilirubin >2×ULN). Downstream safety monitoring systems evaluate incoming patient lab values against these trees without re-reading protocol text; (b) Graded severity threshold ladders. Safety threshold objects encoding escalating action triggers keyed to clinical parameters. Example: Absolute Neutrophil Count carrying three threshold objects (Grade 2: ANC <1.5×10\hat{ }9/L, action “monitor”; Grade 3: ANC <1.0, action “hold_treatment”; Grade 4: ANC <0.5, action “discontinue_and_notify”). Consumed by EDC edit-check configuration and safety dashboard generation; (c) Conditional scheduling rules. Follow-up rule objects encoding post-event visit scheduling logic with trigger event references, visit window arrays (days from trigger with plus/minus tolerance), required service arrays, and termination condition trees. Downstream calendar generation modules execute these to produce patient-specific follow-up schedules; (d) Concomitant therapy evaluation arrays. Medication class objects carrying status classifications (allowed/prohibited/restricted/requires_washout), washout period durations, conditional exception logic, and ATC code references. Consumed by pharmacy systems and concomitant medication review tools; (e) Discontinuation condition trees. Evaluatable criterion objects encoding stopping conditions as Boolean trees (same structure as AESI trees) with criterion type classifications, required action arrays (hold_treatment, notify_sponsor, obtain_consult), and follow-up plan references. Downstream systems execute these against patient data to flag protocol-specified stopping conditions; (f) Eligibility evaluation logic. Inclusion/exclusion criteria encoded as structured parameter-comparator-threshold objects that downstream systems translate to executable database queries against EMR/EHR systems (OMOP CDM field paths, FHIR resource types) for automated patient pre-screening; (g) Cell-level conditional rendering logic. The CPTM data structure includes per-cell option objects (stored at cellOptions in initialState.js) that encode display conditions, NA flags, required-initials flags, and custom annotations. These are consumed by the template rendering engine to produce context-specific operational documents. Each example is “executable” in that downstream systems programmatically evaluate them against data inputs, producing operational outputs without human interpretation of narrative text. They are structured logic objects that function as executable specifications.

### AI Extraction Architecture Implementation

According to one embodiment, the AI-assisted extraction module receives an unstructured clinical trial protocol document and produces a structured protocol data object by applying a comprehensive system prompt that encodes the entire schema definition, field-level constraints, enumeration values, service type taxonomy, and structural rules. The system prompt functions as a constraint on the large language model's output space, limiting outputs to valid structured formats. According to some embodiments, the service type taxonomy comprises fourteen defined types: administration, assessment, ECG, vital signs, laboratory, imaging, biospecimen, informed consent, questionnaire, procedure, concomitant medication, safety monitoring, randomization, and eligibility evaluation. According to another embodiment, the large language model outputs a structured JSON object conforming to the schema, wherein the large language model is confined exclusively to extraction and classification roles within a first tier of a hallucination firewall architecture. The large language model performs entity extraction identifying clinical procedures, drug names, lab panels, and visit schedules, classification assigning each extracted entity to one of the fourteen service types; rule identification parsing safety thresholds, adverse event definitions, concomitant medication rules, and discontinuation criteria into machine-readable logic trees; schedule parsing extracting visit windows, timing constraints, and conditional scheduling rules; and vocabulary normalization producing canonical names that feed deterministic lookup pipelines for clinical terminology standards. According to one example, confidence scoring on extracted elements routes low-confidence extractions to human review queues while high-confidence extractions proceed to validation.

According to various embodiments, every element within the structured protocol data object carries a source mapping comprising a page number, section reference, and excerpt text from the originating protocol document, enabling structural explainability and traceable provenance from any downstream artifact through the structured protocol data object node to the exact protocol language that defines it. According to another embodiment, the AI extraction module within the first tier can be upgraded independently without affecting downstream generation logic, and the deterministic generation modules for electronic data capture, consent documents, calendars, and budgets are independently scoped and independently replaceable, enabling partial platform adoption without requiring the full system. According to one example, for genuinely ambiguous protocol language where the source document does not provide sufficient clarity, the system surfaces the ambiguity explicitly to the human reviewer, forcing resolution at a single point and producing consistent operational interpretation across all participating clinical trial sites.

### Deterministic Generation Module Implementation

According to one embodiment, the deterministic generation module produces clinical and regulatory operational documents by template instantiation and rule application against the locked structured protocol data object, wherein the deterministic generation module contains no artificial intelligence inference components, no neural network processing components, and no probabilistic computation components. According to some embodiments, visit calendar generation uses arithmetic computation on schedule fields within the structured protocol data object, reading service objects with their schedule linkages including visit definitions and timing windows defined as days-from-randomization with plus/minus tolerances, and deterministically computing a patient-facing visit calendar with exact date windows, procedure lists per visit, and duration estimates. According to another embodiment, treatment checklist generation iterates through all service objects scheduled at each visit, reads type-specific fields including dose amounts, administration routes, pre-medication requirements, and timing constraints, and produces a sequential task checklist wherein conditional logic trees from protocol rules are rendered as decision points in the checklist. According to various embodiments, templates are stored as structured objects comprising HTML content, smart field placeholders resolving to data paths within the structured protocol data object, conditional rendering logic, and loop constructs for repeating elements. According to one example, templates are human-authored, version-controlled artifacts that are not embedded in code and not dynamically generated by artificial intelligence.

According to various embodiments, the deterministic generation module supports eligibility-to-EMR mapping wherein eligibility evaluation service objects are mapped via deterministic lookup tables to OMOP CDM tables including condition_occurrence, measurement, drug_exposure, procedure_occurrence, observation, person, and visit occurrence, and to HL7 FHIR R4 resources including Condition, Observation, MedicationRequest, MedicationStatement, Procedure, Patient, AllergyIntolerance, and DiagnosticReport. According to another embodiment, the template library is extensible such that new templates can be authored for different document types, institutional formats, or regulatory jurisdictions, and template versions are recorded alongside structured protocol data object versions for complete audit trail maintenance.

According to another embodiment, the eligibility-to-EMR mapping architecture is extensible to additional target systems including institutional-specific EHR APIs and PCORnet CDM as additional lookup table entries without modifying the core mapping pipeline. According to various embodiments, the deterministic generation module produces byte-identical outputs given the same locked structured protocol data object input, and this determinism property is critical for regulatory compliance verification wherein a sponsor or regulator can verify that clinical documents used at a site were deterministically derived from the approved protocol data.

### Referential Integrity and Dependency Graph Implementation

According to one embodiment, the structured protocol data object implements referential integrity through unique identifiers serving as foreign keys across an object graph encoded in JSON format. According to some embodiments, when any node within the structured protocol data object is modified during AI review or human review phases, the system traces all referential links from that node to downstream artifacts and dependent nodes. According to another embodiment, the referential integrity propagation operates analogously to foreign key cascading in relational databases but operates on a JSON document graph rather than relational tables. According to one example, modifying a laboratory service's vocabulary code propagates to the electronic data capture field configuration for that laboratory test, the safety threshold objects that reference that parameter, the consent language referencing that test, and the grid line (e.g., operational resource requirement) for that procedure. According to various embodiments, the system produces a structured diff showing which artifacts changed and which node modification caused each change, enabling auditable change tracking across the entire artifact set.

According to another embodiment, the scaffold displays dual normal ranges for each clinical parameter comprising protocol-specific thresholds with associated action triggers alongside standard clinical reference ranges, alerting the treating clinician when a protocol-specific threshold is more conservative than the standard clinical threshold. According to one example, a scaffold section for a laboratory assessment displays both the standard clinical reference range for the analyte and the protocol-mandated monitoring threshold with its action trigger such as protocol threshold ANC less than 1.0 times 10 to the ninth per liter triggers treatment hold per the applicable protocol section.

According to various embodiments, multi-parameter procedures such as vital signs assessments generate scaffold sections with sub-entries for each parameter including blood pressure, heart rate, temperature, respiratory rate, and oxygen saturation, with each sub-entry carrying its own expected range, its own LOINC code, and its own data entry field, grouping parameters under the parent procedure heading while maintaining parameter-level structure needed for protocol rules evaluation and electronic data capture data flow.

### Human Validation Gate Implementation

According to one embodiment, the human validation gate presents a side-by-side comparison of extracted data against source protocol text, enabling qualified human reviewers to validate extracted content against the source document. According to some embodiments, the human validation interface displays confidence indicators highlighting low-confidence extractions that require particular attention during review. According to another embodiment, the human validation gate performs structural completeness checks verifying that all required schema sections are populated and cross-reference validation verifying that all referential links resolve to valid target nodes. According to various embodiments, the human validation interface accepts element-level feedback with error classification including missing information, wrong source, hallucinated content, and misinterpreted content categories. According to one example, upon approval by the human validator, the structured protocol data object transitions to a locked state that is irreversible such that the locked structured protocol data object cannot be modified without creating a new version requiring validation.

According to various embodiments, when a locked structured protocol data object contains an error discovered after downstream artifacts have been generated, the system implements an error correction workflow wherein the system copies the locked structured protocol data object, applies the correction in a new draft version, routes the draft through the human validation gate, and upon approval locks the new version and regenerates all downstream artifacts from the new locked version. The erroneous version remains in the audit trail with an error identification record indicating who found the error, when it was found, and what the error was. Downstream artifacts generated from the old version are flagged as superseded, and the system produces a structured comparison showing changes between versions. According to one example, this workflow is analogous to how protocol amendments work in the regulatory context wherein the approved protocol is not edited in place but rather a numbered amendment is issued, and the structured protocol data object architecture enforces this discipline at the data structure level. According to another embodiment, elements below a configurable confidence threshold can be required to receive explicit individual human review before the structured protocol data object can transition to the locked state, rather than permitting batch approval of the entire object, ensuring that low-confidence extractions receive focused attention during the validation process.

### Audit and Regulatory Compliance Implementation

According to one embodiment, the system maintains an append-only audit log capturing version snapshots of the structured protocol data object, reviewer identity, timestamps, change types, and before/after diffs for each modification. According to some embodiments, change types recorded in the audit log include AI-generated changes from the extraction module, AI-review changes from confidence-gated review, human-edit changes from manual corrections, and signoff events from validation approval. According to various embodiments, the deterministic generation architecture in the second tier provides reproducible proof wherein any downstream artifact can be regenerated from the locked structured protocol data object to demonstrate that the artifact was derived through deterministic processes without artificial intelligence inference. According to one example, regulatory auditors can verify artifact provenance by regenerating documents from the locked source and comparing against distributed versions.

According to another embodiment, electronic signatures are tied to individual user accounts for compliance with 21 CFR Part 11 and EU Annex 11 regulatory requirements, and the system maintains complete version history with an immutable audit trail enabling reconstruction of any historical state and providing a traceable chain from source protocol document to locked structured protocol data object to generated document.

### Pipeline Processing Implementation Examples

According to one embodiment, the confidence-gated pipeline for assigning clinical vocabulary identifiers uses a confidence threshold of 0.85, determined empirically to achieve less than a two percent false-positive rate on vocabulary code assignments. According to some embodiments, the AI normalization step outputs disambiguation context including specimen type, method, panel membership, and clinical context alongside the canonical name and confidence signal. According to another embodiment, the system supports local coding for novel biomarkers without equivalents in standard vocabulary databases, enabling protocol-specific identifiers to be assigned when standard codes are unavailable. According to various embodiments, the system performs vocabulary version reconciliation on a biannual release cadence, updating the local vocabulary database when new versions are released and flagging previously assigned codes that may require review under the updated vocabulary. According to one example, the pipeline achieves ninety-two to ninety-five percent accuracy on above-threshold assignments as measured against expert-validated reference sets.

According to another embodiment, the human review workflow for below-threshold results presents a review queue containing the raw protocol text describing the test, the large language model's normalized name and disambiguation context, the top candidate vocabulary codes with match scores and long common names, and options to accept a candidate, reject all candidates and assign manually, or flag for specialist review. Reviewer decisions are captured as structured feedback including the selected code, the reviewer's confidence assessment, and any corrections to the normalized name or disambiguation context, and this feedback feeds a continuous improvement cycle wherein patterns of normalization errors inform prompt engineering updates. According to one example, the vocabulary version reconciliation process flags deprecated codes for review, maps code merges to surviving codes, and surfaces new codes matching previously unmatched tests as potential assignments, with the reconciliation logged and reviewed before structured protocol data objects are updated.

According to one example, a representative randomization service object comprises service identifier svc_rand_001, service type randomization, method stratified block, ratio with treatment arm value 2 and placebo value 1, block sizes of 3 and 6, stratification factors including prior therapy with categories naive and experienced sourced from eligibility assessment and region with categories north America, europe, and asia pacific sourced from site metadata, unblinding rules with emergency unblinding permitted requiring medical monitor approval and documentation of SAE report and unblinding justification and study-level unblinding triggered by DMC recommendation at interim analysis, and IP handling with kit types including KIT_A containing treatment 100 mg at room temperature and KIT_P containing matching placebo at room temperature, dispensing rules triggered by randomization confirmation with sequential kit number sequence, and resupply trigger at 2 kits remaining via automatic depot shipment, with visit reference V1 randomization and source reference page 42 section 5.1.

### Three-Layer Decomposition Implementation

According to one embodiment, interventional entities within the structured protocol data object are decomposed into definition sub-objects, logistics sub-objects, and task sub-objects with explicit referential links between layers. According to one example, a pembrolizumab intervention comprises a definition layer encoding drug identity and weight-based dosing parameters, a logistics layer encoding cold chain storage requirements, preparation procedures, and shipment handling, and a task layer encoding an IV infusion service object with premedication requirements and monitoring parameters. According to some embodiments, referential links between layers use intervention identifiers, intervention references, and logistics references that enable a single definition object to be referenced by multiple logistics and task objects without duplication of definition data. According to another embodiment, combination therapy representation uses multiple definition objects that can share logistics objects or maintain independent logistics configurations depending on handling requirements. According to various embodiments, the three-layer decomposition enables independent versioning of definition, logistics, and task layers, supporting scenarios where logistics requirements change without affecting the intervention definition.

According to various embodiments, the patient journey visualization includes a protocol rules evaluation track as a parallel timeline showing when protocolRules criteria were evaluated against actual patient data, which criteria were triggered, and what actions resulted from each triggered criterion, enabling clinical staff to see the relationship between recorded clinical values and protocol-mandated responses. According to another embodiment, component-level granularity in the patient-hydrated structured protocol data object enables analysis identifying that specific procedure components such as a 4-hour post-dose PK timepoint are systematically missed across the patient population, distinguishing protocol design problems from site compliance problems and informing protocol amendment decisions.

According to one example, the logistics layer is optional for service types where logistics are not applicable, such as questionnaires, assessments, and eligibility evaluations, and service types without logistics simply omit the logistics reference without breaking the three-layer paradigm.

### Site Capability Profiling Implementation

According to one embodiment, the site capability profiling module implements a configurable weighting model with required, preferred, and differentiating tiers for capability dimensions. According to some embodiments, the system generates low-confidence profiles for sites with limited study history on the platform, flagging capability assessments as preliminary when based on fewer than a threshold number of completed studies. According to another embodiment, the system supports bootstrap through manual capability declaration, distinguishing between demonstrated capabilities derived from validated protocol data objects and declared capabilities entered through site self-reporting According to various embodiments, the system generates structured capability gap reports with dimension-by-dimension comparison between protocol requirements and site capabilities, gap classification indicating severity of each identified gap, and suggested remediation actions for addressing capability gaps. According to one example, recency weighting adjusts capability scores based on time since last demonstrated experience, accounting for staff turnover and evolving site capabilities. According to some examples, the feasibility scoring operates through entirely deterministic field comparison with no machine learning inference in the feasibility matching step.

According to another embodiment, the complexity metrics computed from structured protocol data objects include procedure complexity comprising PK timepoint density, biospecimen collection type diversity, imaging modality mix, and assessment instrument complexity; protocol rule complexity comprising logic tree depth and node count, safety threshold parameter count, and concomitant medication rule count; and operational complexity comprising treatment arm count, visit type count, visit window tightness, and amendment frequency. According to various embodiments, the capability gap report is provided in a structured data format enabling programmatic filtering and sorting by sponsors, and principal investigator-specific capability views are optionally available alongside the site-level primary profile unit.

Patient-Hydrated CPTM (pCPTM) Implementation

According to one embodiment, the patient-hydrated instance of the structured protocol data object comprises delta fields with a deviation type enumeration including timing deviation, value deviation, omission, addition, and substitution categories. According to some embodiments, each delta field includes magnitude indicating the size of deviation, direction indicating whether the deviation exceeded or fell below expected values, and severity classification categorizing deviations as minor, major, or critical. According to another embodiment, the patient-hydrated instance supports per-component status arrays for partial completion scenarios where some but not all elements of a multi-part procedure are completed. According to various embodiments, population-level deviation detection operates with configurable thresholds, generating alerts when deviation rates exceed fifteen percent or rule trigger rates exceed ten percent across the patient population. According to one example, amendment signal generation produces structured recommendations when population-level deviation patterns suggest protocol modifications may be warranted. According to some examples, integration paths for patient data include electronic data capture mapping, HL7 FHIR ingestion, and API-based capture from clinical trial management systems. According to another example, patient journey visualization renders as a color-coded timeline showing expected versus actual procedure completion across the study schedule.

According to another embodiment, the deviation classification uses a three-tier severity model wherein minor deviations are outside the expected window but within tolerance with no rule trigger, major deviations violated a protocol requirement or triggered a threshold or were omitted entirely, and critical deviations impact patient safety or data integrity. According to various embodiments, the automated classification is computed from delta fields and applicable protocol rule objects, and a human reviewer can confirm or override the automated classification. According to one example, the closed-loop data flow operates wherein the structured protocol data object generates a scaffold, the scaffold generates an EHR template, the clinician completes the note, data is extracted via EHR API, matched to service nodes via embedded identifiers, the patient-hydrated instance is populated, and deviation evaluation runs automatically.

### Three-Dimensional Versioning Implementation

According to one embodiment, the version identifier format comprises three components: content version, schema version, and amendment version, formatted as content.schema amendment. According to some embodiments, content version increments on data modification within the structured protocol data object, schema version increments on platform schema updates affecting the data model, and amendment version increments on formal sponsor amendments to the protocol. According to another embodiment, the system stores full JSONB snapshots for each version, enabling retrieval of the complete structured protocol data object state at any historical version. According to various embodiments, temporal matching for compliance evaluation identifies the specific protocol version in effect at the time of a clinical event, enabling deviation assessment against the protocol state that governed the event rather than the current protocol state. According to one example, country-specific amendment numbering is supported via site instance jurisdiction mapping, enabling different amendment version sequences for different regulatory jurisdictions participating in a global study.

### EHR Clinical Note Scaffold Implementation

According to one embodiment, the EHR clinical note scaffold generation targets HL7 FHIR DocumentReference, HL7 CDA, and structured HTML output formats for integration with electronic health record systems. According to some embodiments, the scaffold is organized by visit with sections for each procedure containing expected parameters, normal ranges derived from both protocol rules threshold objects and standard clinical reference ranges, structured data entry fields, and protocol reference links. According to another embodiment, metadata blocks within the scaffold include the service identifier from the structured protocol data object, the version identifier, and a deep link URL enabling navigation from the clinical note back to the source protocol procedure definition. According to various embodiments, bidirectional traceability enables patient-hydrated instance population from completed clinical notes, supporting automated capture of actual procedure data from EHR documentation. According to one example, free-text narrative fields within the scaffold accommodate clinical observations not subject to automated evaluation, preserving clinician documentation flexibility while maintaining structured data capture for protocol-defined measurements.

According to various embodiments, the target clinician workflow presents the pre-populated scaffold in the EHR before the patient visit, and for sites without direct EHR template integration, a parallel document mode generates a standalone document that the clinician completes and uploads. According to one example, the complete closed loop operates wherein the structured protocol data object generates the scaffold, the scaffold generates the EHR template, the clinician completes the note, data is extracted via EHR API, matched to service nodes via embedded identifiers, the patient-hydrated instance is populated, and deviation evaluation runs automatically against the protocol rules.

eTMF Taxonomy Implementation

According to one embodiment, the trial master file taxonomy generation implements a two-level mapping engine wherein Level 1 maps element types from the structured protocol data object to TMF zones via a static lookup table and Level 2 applies study-specific configuration refinement based on study metadata. According to some embodiments, the system categorizes TMF documents into three categories: always required documents present in every study index regardless of protocol content, protocol-derived documents included based on analysis of the structured protocol data object, and optional documents included based on study-specific configuration by the user. According to another embodiment, timing derivation uses the visit schedule and study milestones from the structured protocol data object, computing expected submission timing for each document type relative to protocol-defined events. According to various embodiments, regulatory overlay tables for country-specific requirements are activated via site instance jurisdiction, enabling jurisdiction-specific TMF requirements to supplement the base TMF Reference Model. According to one example, export formats support Veeva Vault TMF via Vault Loader-compatible CSV, MasterControl, and Montrium eTMF systems through system-specific import schema adapters.

### IRT Configuration Implementation

According to one embodiment, the randomization service object schema within the structured protocol data object comprises method specifying the randomization algorithm, ratio specifying arm allocation ratios, block sizes specifying permuted block parameters, stratification factors specifying stratification variables and levels, unblinding rules specifying conditions for emergency unblinding, and IP handling sections specifying drug supply management parameters. According to some embodiments, kit type definitions, dispensing rules, resupply logic, and temperature excursion handling parameters are extracted from IP handling service objects to configure drug supply management within the IRT system. According to another embodiment, the system generates a vendor-neutral configuration format with vendor-specific format adapters for translation to specific IRT system schemas. According to various embodiments, the system supports complex randomization schemes including response-adaptive randomization, Bayesian adaptive designs, and platform trials with nested randomization objects representing sub-study randomization within master protocol structures. According to one example, the generated IRT configuration file is produced through deterministic mapping of extracted parameters to the IRT system schema without artificial intelligence inference at the configuration generation step.

According to various embodiments, the unblinding rules section of the randomization service object encodes conditions under which individual patient unblinding is permitted, approval requirements specifying who must authorize unblinding, documentation requirements including SAE reports and unblinding justification forms, and process steps including call sequences and post-unblinding reporting obligations. According to another embodiment, study-level unblinding is encoded separately with trigger events including DMC recommendations, scope specifying which arms and endpoints, and IRT access controls specifying who has unblinding access under what conditions. According to one example, drug supply logistics are captured in the IP handling section including depot definitions, site-level inventory management computed from enrollment projections and visit frequency, expiration date tracking, and temperature monitoring requirements, and amendment propagation recomputes per-site inventory targets when dosing schedules change.

### Protocol Rules Implementation Examples

According to one embodiment, the adverse event of special interest logic trees support arbitrary nesting depth as recursive data structures, and circular references are structurally impossible because each node is a value object rather than a reference to another node, making the tree a directed acyclic graph by construction. According to some embodiments, the system detects conflicting rules when multiple protocol rule objects reference the same clinical parameter with overlapping conditions, and the system flags conflicts during human review wherein conflict resolution is a clinical judgment that belongs in the human validation gate rather than in automated logic. According to another embodiment, each safety threshold object carries a CTCAE version field identifying the grading standard, and protocol-specific grading that deviates from CTCAE is handled through override fields carrying both the standard CTCAE grade and the protocol-specific override with a flag indicating deviation from the standard, enabling dual-coding for both standard CTCAE-based safety reporting and protocol-specific action triggers. According to one example, in practice, clinical protocol adverse event of special interest definitions rarely exceed three to four levels of nesting, but the schema imposes no hard limit on depth. According to another embodiment, a dosage-dependent concomitant therapy rule for acetaminophen carries status restricted with a condition object specifying a daily dose parameter with comparator greater than and threshold of two grams, encoding acetaminophen allowed up to two grams per day and prohibited above that threshold.

According to various embodiments, dosage-dependent concomitant therapy rules are represented as condition objects with parameter fields specifying dosage thresholds, and timing-dependent interactions use temporal condition objects with washout period fields and timing relationship fields specifying whether restrictions apply to concurrent use, sequential use within a window, or cumulative exposure over a period. According to another embodiment, the discontinuation criteria array is ordered by clinical priority with each criterion carrying a priority field, and when multiple criteria are simultaneously satisfied, the system applies the highest-priority criterion's action sequence while logging all satisfied criteria for regulatory reporting purposes. According to one example, jurisdiction-specific reproductive safety requirements are encoded through a jurisdiction overrides array on reproductive safety requirement objects with ISO 3166-1 country codes, activated through the site instance layer to enable a single protocol to encode all jurisdictional variations in machine-readable form.

According to one example, a representative protocolRules section populated with clinical data comprises: an aesiDefinitions array containing a Hy's Law Criteria object with a logic tree having an OR operator at the root, a first child with an AND operator containing ALT greater than 3 times ULN (LOINC 1742-6) and total bilirubin greater than 2 times ULN (LOINC 1975-2), and a second child specifying ALT greater than 5 times ULN, with source reference to page 87 section 8.3.2; a safetyThresholds array containing an Absolute Neutrophil Count object (LOINC 26499-4) with Grade 2 at less than 1.5 times 10 to the ninth per liter with action monitor, Grade 3 at less than 1.0 with action hold treatment, and Grade 4 at less than 0.5 with action discontinue and notify, referencing CTCAE version 5.0 and source page 92 section 8.4.1; a concomitantTherapyRules array containing a Strong CYP3A4 Inhibitors object with ATC prefix J02AC, status prohibited, washout period 14 days, and an exception for life-threatening indication requiring medical monitor approval, with source page 68 section 6.5.2; a discontinuationCriteria array containing a Hepatotoxicity Discontinuation object of type protocol specified with a condition tree encoding ALT greater than 10 times ULN OR sustained ALT greater than 5 times ULN for 14 days, with actions hold treatment, notify sponsor within 24 hours, and obtain hepatology consult, and a follow-up plan reference to safety follow-up hepatotoxicity; a followUpRules array containing a safety follow-up hepatotoxicity object triggered by hepatotoxicity discontinuation with schedule windows at 7 days requiring hepatic panel and physical exam and at 30 days requiring hepatic panel, with termination condition of ALT less than or equal to 1 times ULN stable for 30 days; and a reproductiveSafetyRequirements array containing an object for females of childbearing potential requiring highly effective contraception starting 28 days before first dose through 90 days after last dose, serum hCG testing every 28 days within 72 hours before dosing, and action on positive of immediate discontinuation with notification to PI, sponsor safety, and IRB within 24 hours.

### Procedure-Based Paradigm Implementation Examples

According to one embodiment, variable timing windows are encoded per-visit within each service object's schedule linkage array, enabling different timing tolerances for different visits of the same procedure. According to some embodiments, conditional procedures are represented through a condition logic field on service objects using the same Boolean logic tree structure as protocol rules, with a distinction between scheduled-conditional procedures that appear on the schedule but execute only when conditions are met and event-triggered procedures that do not appear on the schedule but are triggered by clinical events. According to another embodiment, multi-standard representation enables panel-level and component-level representations to be co-resident on the same service object, with downstream modules selecting the appropriate representation for the target system.

According to one example, a comprehensive metabolic panel at Visit 3 of a Phase II oncology trial carries schedule linkage with visit references to V3, V5, V8, and EOT with target day 29 and window minus 3 and window plus 3, terminology alignment with LOINC code 24323-8, CDISC domain LB, SNOMED code 14768001, and NIH CDE identifier CDE2943570, safety rule references to threshold ALT hepatotoxicity, threshold creatinine renal, and AESI Hy's Law, and source reference to page 45 section 7.2.1, from which the system deterministically generates five downstream artifacts including EDC field configuration, patient visit calendar line, val. line item with CPT 80053, consent form language, and safety monitoring trigger configurations.

According to various embodiments, procedures with variable frequency such as weekly for the first eight weeks then every four weeks are represented as conditional scheduling logic on the service object with the visit reference array including all applicable visits and scheduling conditions encoding the frequency change rules. According to another embodiment, procedures spanning multiple calendar days such as 24-hour urine collection or 72-hour Holter ECG monitoring are represented with duration fields on the service object, and the calendar generator accounts for these durations when computing visit timing and avoiding scheduling conflicts. According to one example, each deterministic generation module reads the vocabulary identifier relevant to its target system wherein the EDC module reads the CDISC domain mapping, another module reads the CPT or HCPCS code, the EHR integration reads the LOINC code, and the regulatory submission module reads the NIH CDE identifier.

### Multi-Standard Cross-Reference Implementation Examples

According to one embodiment, each vocabulary entry within a service object carries mapping confidence and mapping note fields for handling inexact cross-vocabulary mappings where a one-to-one correspondence does not exist between standards. According to some embodiments, cross-vocabulary consistency validation operates at a structural level verifying that mapped codes belong to the same clinical concept category and at a semantic level using UMLS Metathesaurus mappings to verify that codes represent the same clinical meaning. According to another embodiment, the vocabulary structure is extensible as a key-value architecture enabling addition of new standards such as ICD-10 or RxNorm without affecting existing vocabularies or downstream generation modules.

According to one example, a C-Reactive Protein service object carries service identifier svc_lab_crp_001, service type laboratory, name C-Reactive Protein, and vocabulary with LOINC code 1988-5, CDISC domain LB, CDISC test code CRP, SNOMED code 55235003, NIH CDE identifier CDE2943826, and CPT code 86140, with visit references V1, V3, V5, V8, and EOT, wherein each downstream module reads the relevant code such that the EDC module uses the CDISC domain and test code, another module uses the CPT code, the EHR integration uses the LOINC code, the regulatory submission module uses the NIH CDE identifier, and clinical documentation uses the SNOMED code.

According to various embodiments, inconsistencies between vocabulary standards are flagged during the validation phase and are not automatically resolved, and the system surfaces conflicts with relevant vocabulary definitions and known cross-references for human review. According to another embodiment, adding a new vocabulary standard is a schema extension rather than an architectural change because vocabularies are properties of the procedure object rather than separate domain-specific data structures.

According to one example, specific TMF mapping associations include informed consent service objects mapping to TMF Zone 8 for IRB and ethics committee approvals, laboratory service objects mapping to TMF Zone 4 for central lab agreements and TMF Zone 5 for lab normal range documentation, randomization service objects mapping to TMF Zone 4 for randomization scheme documentation, and eligibility evaluation service objects mapping to TMF Zone 5 for screening and enrollment documentation.

According to another embodiment, non-protocol-derived documents including financial agreements, confidentiality agreements, and regulatory correspondence are included as standing requirements in every study index regardless of protocol content, ensuring the TMF taxonomy captures both protocol-derived and universally required documentation.

According to one example, a central imaging worked example demonstrates that an imaging service object with modality CT, reading method central independent review, imaging vendor requirement true, and RECIST version 1.1 maps through the TMF derivation engine to generate requirements for Imaging Charter, Independent Review Charter, Reader Training documentation, Central Imaging Lab Agreement, Imaging Transfer Specifications, Image Quality Review Procedures, and RECIST 1.1 Training Documentation, with each derived artifact appearing in the study-specific TMF index with the originating CPTM element, expected timing computed from the visit schedule, and responsible party assignment.

### Biospecimen Dual-Identity Implementation Examples

According to one embodiment, the unified handling section reconciles specimens from both scheduled collection and procedure byproduct paths by identifying when specimens from different sources share handling requirements and consolidating logistics configurations, reducing duplication in operational documents. According to some embodiments, when a protocol amendment elevates a byproduct to a scheduled collection, the tissue fields on the originating procedure are preserved for historical versions and a new biospecimen service object is created in the amended version with its own schedule linkage, while the handling configuration can remain the same to maintain logistics continuity. According to another embodiment, biospecimen service objects carry the full service object field set including schedule linkage, collection specifications, vocabulary alignment, protocol rules references, and handling logistics references, while tissue byproduct fields carry a subset including specimen type, fixative, quantity requirements, and handling reference without independent schedule linkage or vocabulary codes.

According to one example, a PK blood draw as a scheduled collection carries service identifier svc_bio_pk_v3_001, service type biospecimen, name PK Blood Draw 4 hr Post-Dose, collection type blood, volume 5 mL, tube type K2 EDTA, processing instructions including centrifuge at 3000 rpm for 10 minutes at 4 degrees Celsius with 2 aliquots, handling reference pkSampleHandling, visit references V3, and timing within visit 4 hours post-dose with tolerance 15 minutes. According to another example, a core needle biopsy as a procedure with tissue byproduct carries service identifier svc_proc_biopsy_001, service type procedure, name Core Needle Biopsy Liver, SNOMED code 44578007, CPT code 47000, and tissue byproduct fields including specimen type tissue, tissue type liver core, fixative 10 percent neutral buffered formalin, cores minimum 3 target 5, and handling reference tissueSampleHandling, with visit references V1 screening.

According to various embodiments, biospecimen service objects appear as independent rows on the Schedule of Activities because specimen collection is the clinical activity, while tissue byproducts do not appear as independent rows because the procedure is the clinical activity and the specimen is a consequence. According to another embodiment, the unified handling section defines end-to-end specimen logistics including collection requirements, processing steps, storage conditions, shipping specifications, central lab routing, and chain-of-custody documentation requirements.

### Canonical/Site-Instance Implementation Examples

According to one embodiment, the field separation principle provides that if data comes from the protocol it is canonical and if data comes from the site it is site-instance, and no site-instance field can override a canonical protocol rule. According to some embodiments, the conflict prevention architecture enforces at the schema level that canonical fields are not writable from the site instance layer, and genuine conflicts must be resolved through the sponsor via protocol deviation request or formal amendment rather than through site-level override. According to another embodiment, site-specific document generation operates by Tier 2 generation modules receiving both the canonical structured protocol data object and the site instance layer, resolving protocol-level content from the canonical layer and site-specific content from the site instance layer to produce documents that are site-specific but canonically consistent.

According to various embodiments, the canonical layer contains all service objects, visit definitions, protocol rules, intervention definitions, schedule logic, and study metadata including phase, indication, sponsor identity, and IND or CTA numbers. According to another embodiment, the site instance layer contains local lab reference ranges, local IRB or ethics committee identifiers and approval dates, local investigator and staff assignments, local form numbering conventions, site-specific contact information, local regulatory identifiers, local vendor contacts, and site-specific procedure notes.

According to one embodiment, site instances carry their own version sequence independent of the canonical structured protocol data object version, and when the canonical version changes on amendment, all site instances are flagged for reconciliation review, but the site instance does not automatically update. According to some embodiments, the canonical layer is immutable after lock, and site instances reference the canonical layer by version identifier rather than mutable pointer, ensuring that a site instance created against a specific canonical version always references that version. According to another embodiment, the canonical audit trail is independent of site instances, and no site-level action can modify or corrupt the canonical audit trail, enabling sponsor-level auditability while preserving site-level autonomy.

According to one example, a representative version metadata record comprises version identifier 2.3.1, content version 2, schema version 3, amendment version 1, a complete structured protocol data object snapshot, change type human edit, actor identifier, timestamp, change description indicating updated ANC safety threshold from 1.0 to 0.75 per protocol clarification letter, amendment metadata including amendment number 1, sponsor date, effective date, and regulatory reference, parent version identifier 2.3.0, and a diff object containing modified arrays listing changed node paths, added arrays listing new nodes, and removed arrays listing deleted nodes.

### JSON Encoding Example

Example (abridged, showing representative objects for types (e.g., six)):

{ “protocolRules”: { “aesiDefinitions”: [{ “name”: “Hy's Law Criteria”, “logicTree”: {
“operator”: “OR”, “children”: [ { “operator”: “AND”, “children”: [ {
“parameterRef”: “LOINC:1742-6”, “comparator”: “greater_than”, “threshold”: { “value”: 3,
“unit”: “xULN” } }, { “parameterRef”: “LOINC:1975-2”, “comparator”: “greater_than”,
“threshold”: { “value”: 2, “unit”: “xULN” } } ]}, { “parameterRef”: “LOINC:1742-6”,
“comparator”: “greater_than”, “threshold”: { “value”: 5, “unit”: “xULN” } } ] },
“sourceRef”: { “page”: 87, “section”: “8.3.2” } }], “safetyThresholds”: [{ “parameterId”:
“LOINC:26499-4”, “parameterName”: “Absolute Neutrophil Count”, “grades”: [ {
“grade”: 2, “comparator”: “less_than”, “value”: 1.5, “unit”: “10\hat{ }9/L”, “action”: “monitor” }, {
“grade”: 3, “comparator”: “less_than”, “value”: 1.0, “unit”: “10\hat{ }9/L”, “action”: “hold_treatment”
}, { “grade”: 4, “comparator”: “less_than”, “value”: 0.5, “unit”: “10\hat{ }9/L”, “action”:
“discontinue_and_notify” } ], “ctcaeVersion”: “5.0”, “sourceRef”: { “page”: 92, “section”:
“8.4.1” } }], “concomitantTherapyRules”: [{ “medicationClass”: “Strong CYP3A4
Inhibitors”, “atcPrefix”: “J02AC”, “status”: “prohibited”, “washoutPeriod”: { “value”: 14,
“unit”: “days” }, “exceptions”: [{ “condition”: “life_threatening_indication”, “status”:
“restricted”, “requiresApproval”: “medical_monitor” }], “sourceRef”: { “page”: 68, “section”:
“6.5.2” } }], “discontinuationCriteria”: [{ “name”: “Hepatotoxicity Discontinuation”,
“criterionType”: “protocol_specified”, “conditionTree”: { “operator”: “OR”, “children”:
[ { “parameterRef”: “LOINC:1742-6”, “comparator”: “greater_than”, “threshold”: { “value”:
10, “unit”: “xULN” } }, { “operator”: “AND”, “children”: [ { “parameterRef”:
“LOINC:1742-6”, “comparator”: “greater_than”, “threshold”: { “value”: 5, “unit”: “xULN” } },
{ “duration”: { “value”: 14, “unit”: “days” }, “condition”: “sustained” } ]} ] },
“actionRequired”: [“hold_treatment”, “notify_sponsor_within_24h”,
“obtain_hepatology_consult”], “followUpPlan”: { “ref”: “safety_follow_up_hep” } }],
“followUpRules”: [{ “id”: “safety_follow_up_hep”, “triggerEvent”:
“hepatotoxicity_discontinuation”, “followUpType”: “safety_follow_up”,
“scheduleWindows”: [ { “daysFromTrigger”: 7, “toleranceDays”: 2, “requiredServices”:
[“lab_hepatic_panel”, “assessment_physical_exam”] }, { “daysFromTrigger”: 30,
“toleranceDays”: 5, “requiredServices”: [“lab_hepatic_panel”] } ], “terminationCondition”:
{ “operator”: “AND”, “children”: [ { “parameterRef”: “LOINC:1742-6”, “comparator”:
“less_than_or_equal”, “threshold”: { “value”: 1, “unit”: “xULN” } }, { “duration”: { “value”:
30, “unit”: “days” }, “condition”: “stable” } ]} }], “reproductiveSafetyRequirements”: [{
“affectedSex”: “female”, “fertilityStatus”: “of_childbearing_potential”,
“contraceptionRequirements”: [ { “methodType”: “highly_effective”, “startTiming”:
“28_days_before_first_dose”, “duration”: “90_days_after_last_dose” } ],
“pregnancyTestSchedule”: { “testType”: “serum_hCG”, “frequency”: “every_28_days”,
“timingRelativeToDosing”: “within_72h_before” }, “actionOnPositive”: { “action”:
“immediate_discontinuation”, “notification”: [“PI”, “sponsor_safety”, “IRB_within_24h”] } }]

### Example Embodiments

A system for data management, the system comprising: at least one processor operatively connected to a memory, the at least one processor when executing configured to: accept input of unstructured or multi-format clinical trial data; process the unstructured or multi-format data into a compressed structured data format, wherein the compressed data format is organized on procedure-based associations linking procedure-based information to associated clinical trial data; wherein the operations executed to process the unstructured or multi-format data include: instantiate a machine learning model configured to analyze clinical protocol data to identify procedure-based information and associated clinical trial data in response to input of unstructured or multi-format clinical trial data; and output the compressed data format, including respective procedure, procedure-based associations, and associated clinical trial data.

The system of any embodiment, wherein the at least one processor is configured to lock data objects to prevent modification responsive to validation (e.g., thereby prevent introduction of unvalidated AI-based data or unvalidated AI-based information), wherein the at least one processing is configured to enable modification of locked data objects via propagation functions, wherein the propagation functions are configured to require validation before execution (e.g., validation required before propagation of any modification on locked data, updates optionally trigger updating of validation information, etc.), wherein the operations executed to process the unstructured or multi-format data include executing a plurality of rules to identify procedure-based information in unstructured or a plurality of non-homogeneous data sources (e.g., including unstructured text, unformatted text, or natural language data sources, etc.), wherein the executing the plurality of rules includes mapping identified procedures to associated clinical trial data, wherein the compressed structured data format comprises a procedure-based paradigm in which a clinical procedure object serves as a primary organizing entity, and includes at least a node storing executable code for downstream systems, wherein each clinical procedure object simultaneously comprises schedule linkage to one or more visit definitions with protocol-defined timing windows, vocabulary identifiers from a plurality of independent clinical terminology standards, references to applicable protocol rule objects, and a cross-reference index to an originating protocol document section, or wherein the vocabulary identifiers comprise identifiers from at least two of LOINC, CDISC, NIH CDE, and SNOMED co-resident on the clinical procedure object.

The system of any embodiment, wherein the clinical procedure object conforms to one of a plurality of defined service types comprising administration, assessment, ECG, vital signs, laboratory, imaging, biospecimen, informed consent, questionnaire, procedure, concomitant medication, safety monitoring, randomization, and eligibility evaluation.

The system of any embodiment, wherein the at least one processor is further configured to automatically generate a plurality of downstream operational artifacts from the compressed structured data format, wherein the plurality of downstream operational artifacts comprises at least one of treatment checklists, grids, patient calendars, informed consent form drafts, EDC templates, and pharmacy dispensation schedules, wherein modification of a procedure object in the compressed structured data format propagates to all derived artifacts referencing the procedure object, or wherein the machine learning model comprises a large language model configured to extract structured data fields from the unstructured or multi-format clinical trial data and to extract conditional logic including dose thresholds, treatment stopping criteria, and branching conditions.

A method for data management, the method comprising: accepting, by at least one processor, input of unstructured or multi-format clinical trial data; processing the unstructured or multi-format data into a compressed structured data format, wherein the compressed data format is organized on procedure-based associations linking procedure-based information to associated clinical trial data; instantiating a machine learning model configured to analyze clinical protocol data to identify procedure-based information and associated clinical trial data; and outputting the compressed data format including respective procedure, procedure-based associations, and associated clinical trial data.

The method of any embodiment, comprising locking data objects to prevent modification responsive to validation (e.g., thereby prevent introduction of unvalidated AI-based data or unvalidated AI-based information), or further comprising enabling modification of locked data objects via propagation functions, wherein the propagation functions are configured to require validation before execution (e.g., validation required before propagation of any modification on locked data, updates optionally trigger updating of validation information, etc.).

The method of any embodiment, further comprising executing a plurality of rules to identify procedure-based information in unstructured or a plurality of non-homogeneous data sources, wherein executing the plurality of rules includes mapping identified procedures to associated clinical trial data, further comprising automatically generating a plurality of downstream operational artifacts from the compressed structured data format, wherein the plurality of downstream operational artifacts comprises at least one of treatment checklists, grids, patient calendars, informed consent form drafts, EDC templates, and pharmacy dispensation schedules, further comprising distributing the compressed structured data format to a plurality of clinical trial sites via an application programming interface, or wherein the compressed structured data format implements a cross-reference architecture in which procedures link to associated data elements through reference identifiers rather than embedded copies, and includes at least a node storing executable code for downstream systems.

A non-transitory computer-readable storage medium storing processor-executable instructions that, when executed by at least one processor, cause the at least one processor to: accept input of unstructured or multi-format clinical trial data; process the unstructured or multi-format data into a compressed structured data format organized on procedure-based associations linking procedure-based information to associated clinical trial data; instantiate a machine learning model configured to analyze clinical protocol data to identify procedure-based information and associated clinical trial data; and output the compressed data format including respective procedure, procedure-based associations, and associated clinical trial data.

The medium of any embodiment, wherein the instructions cause the at least one processor to lock data objects to prevent modification responsive to validation (e.g., thereby prevent introduction of unvalidated AI-based data or unvalidated AI-based information), wherein the instructions cause the at least one processor to enable modification of locked data objects via propagation functions, wherein the propagation functions are configured to require validation before execution (e.g., validation required before propagation of any modification on locked data, updates optionally trigger updating of validation information, etc.).

The medium of any embodiment, wherein the processor-executable instructions further cause the at least one processor to execute a plurality of rules to identify procedure-based information and map identified procedures to associated clinical trial data, wherein the processor-executable instructions further cause the at least one processor to automatically generate a plurality of downstream operational artifacts from the compressed structured data format comprising at least one of treatment checklists, grids, patient calendars, informed consent form drafts, EDC templates, and pharmacy dispensation schedules.

A system for representing clinical trial operational safety logic, the system comprising: at least one processor operatively connected to a memory, the at least one processor when executing configured to: receive clinical trial protocol data containing safety rules expressed in natural language; generate a structured protocol data object comprising a protocol rules (e.g., protocolRules) section, wherein the protocol rules (e.g., protocolRules) section includes: at least one of: adverse event logic tree objects encoding Boolean evaluation criteria with nested operator nodes, clinical parameter references, comparators, threshold values, and child node arrays; safety threshold objects encoding graded severity monitoring criteria with clinical parameter identifiers conforming to an external vocabulary standard, action triggers, or severity grades; and concomitant therapy rule arrays encoding medication class restrictions with status classifications including at least one of: allowed, prohibited, and restricted designations, washout period durations, or conditional exception logic; and store the structured protocol data object in a machine-readable format enabling computational evaluation of the safety rules against patient clinical data.

The system of any embodiment, wherein the structured data object is accessed, and a care recipient is treated based on, at least in part, the parameters encoded in the structured data object (e.g., based on, at least in part, executable logic/rules etc.). The system of any embodiment, wherein the protocol rules section further includes discontinuation criteria objects encoding evaluatable condition trees with criterion type classifications, required action arrays, and follow-up plan references or the criterion type classifications include at least one of decision status (e.g., investigator_decision), protocol designation (e.g., protocol_specified), sponsor metadata (e.g., sponsor_directed), or patient metadata (e.g., patient_withdrawal) designations.

The system of any embodiment, wherein the protocol rules (e.g., protocolRules) section further includes discontinuation criteria objects encoding evaluatable condition trees with criterion type classifications, required action arrays, and follow-up plan references. The system of any embodiment, wherein the criterion type classifications include investigator_decision, protocol_specified, sponsor_directed, and patient_withdrawal designations. The system of any embodiment, wherein the protocol rules (e.g., protocolRules) section further includes follow-up rule objects encoding post-event clinical scheduling obligations with trigger event references, visit window arrays, required service arrays, and termination condition trees. The system of any embodiment, wherein the trigger event references include SAE, treatment_discontinuation, and study_completion designations. The system of any embodiment, wherein the protocol rules (e.g., protocolRules) section further includes reproductive safety requirement objects encoding sex-specific contraception mandates, pregnancy testing schedules, action-on-positive logic, and partner requirement specifications. The system of any embodiment, wherein the adverse event logic tree objects encode Hy's Law criteria as a Boolean logic tree with an OR operator at a root node, wherein a first child node specifies ALT greater than five times an upper limit of normal, and a second child node contains an AND operator with child nodes specifying ALT greater than three times the upper limit of normal and total bilirubin greater than two times the upper limit of normal. The system of any embodiment, wherein the safety threshold objects create a graded severity ladder for a clinical parameter, wherein multiple safety threshold objects for the clinical parameter encode different severity grades with corresponding action triggers.

The system of any embodiment, wherein the action triggers include hold_treatment, notify_PI, dose_reduce, and discontinue designations. The system of any embodiment, wherein the concomitant therapy rule arrays include medication class identifiers conforming to ATC codes, and wherein the status classifications further include a requires_washout designation. The system of any embodiment, wherein the external vocabulary standard comprises LOINC codes identifying clinical measurements.

A method for representing clinical trial operational safety logic, the method comprising: receiving, by at least one processor, clinical trial protocol data containing safety rules expressed in natural language; generating, by the at least one processor, a structured protocol data object comprising a protocol rules (e.g., protocolRules) section, wherein the protocol rules (e.g., protocolRules) section includes: adverse event logic tree objects encoding Boolean evaluation criteria with nested operator nodes, clinical parameter references, comparators, threshold values, and child node arrays; safety threshold objects encoding graded severity monitoring criteria with clinical parameter identifiers, action triggers, and severity grades; and concomitant therapy rule arrays encoding medication class restrictions with status classifications, washout period durations, and conditional exception logic; and storing the structured protocol data object in a machine-readable format enabling computational evaluation of the safety rules.

The method of any embodiment, wherein the protocol rules (e.g., protocolRules) section further includes discontinuation criteria objects encoding evaluatable condition trees with criterion type classifications, required action arrays, and follow-up plan references. The method of any embodiment, wherein the criterion type classifications include investigator_decision, protocol_specified, sponsor_directed, and patient_withdrawal designations. The method of any embodiment, wherein the protocol rules (e.g., protocolRules) section further includes follow-up rule objects encoding post-event clinical scheduling obligations with trigger event references, visit window arrays, required service arrays, and termination condition trees. The method of any embodiment, wherein the protocol rules (e.g., protocolRules) section further includes reproductive safety requirement objects encoding sex-specific contraception mandates, pregnancy testing schedules, action-on-positive logic, and partner requirement specifications. The method of any embodiment, wherein the safety threshold objects create a graded severity ladder for a clinical parameter, wherein multiple safety threshold objects for the clinical parameter encode different severity grades with corresponding action triggers including hold_treatment, notify_PI, dose_reduce, and discontinue designations.

A non-transitory computer-readable storage medium storing processor-executable instructions that, when executed by at least one processor, cause the at least one processor to: receive clinical trial protocol data containing safety rules expressed in natural language; generate a structured protocol data object comprising a protocol rules (e.g., protocolRules) section including adverse event logic tree objects, safety threshold objects, and concomitant therapy rule arrays in machine-readable format; and store the structured protocol data object enabling computational evaluation of the safety rules against patient clinical data.

The medium of any embodiment, wherein the protocol rules (e.g., protocolRules) section further includes discontinuation criteria objects encoding evaluatable condition trees with criterion type classifications including investigator_decision, protocol_specified, sponsor_directed, and patient_withdrawal designations, required action arrays, and follow-up plan references. The medium of any embodiment, wherein the protocol rules (e.g., protocolRules) section further includes follow-up rule objects encoding post-event clinical scheduling obligations with trigger event references including SAE, treatment_discontinuation, and study_completion designations, visit window arrays, required service arrays, and termination condition trees.

A system for organizing clinical trial protocol data, the system comprising: at least one processor operatively connected to a memory, the at least one processor when executing configured to: generate a structured protocol data object wherein a clinical procedure object serves as a primary organizing entity, each clinical procedure object simultaneously comprising: schedule linkage to one or more visit definitions with protocol-defined timing windows; vocabulary identifiers from a plurality of independent clinical terminology standards co-resident on the clinical procedure object, the plurality of independent clinical terminology standards including at least two of LOINC, CDISC, NIH CDE, and SNOMED; references to applicable protocol rule objects encoding safety, monitoring, and compliance logic; a cross-reference index to an originating protocol document section; and a deterministic anchoring point from which a plurality of downstream operational artifacts are generated; wherein modification of the clinical procedure object propagates to all derived artifacts referencing the clinical procedure object.

The system of any embodiment, wherein the clinical procedure object conforms to one of a plurality of defined service types comprising administration, assessment, ECG, vital signs, laboratory, imaging, biospecimen, informed consent, questionnaire, procedure, concomitant medication, safety monitoring, randomization, and eligibility evaluation. The system of any embodiment, wherein each service type has a type-specific field schema extending a common base schema. The system of any embodiment, wherein the vocabulary identifiers comprise a LOINC code identifying a clinical measurement, a CDISC domain tag for SDTM mapping, an NIH CDE reference for cross-study harmonization, and a SNOMED procedure code. The system of any embodiment, wherein the protocol rule objects comprise adverse event logic tree objects encoding Boolean evaluation criteria.

The system of any embodiment, wherein the protocol rule objects comprise safety threshold objects encoding graded severity monitoring criteria with action triggers. The system of any embodiment, wherein the downstream operational artifacts comprise at least one of EDC configurations, CRF form builds, informed consent documents, treatment checklists, patient visit calendars, grids, and regulatory submission templates.

The system of any embodiment, wherein the cross-reference index enables traceability from the clinical procedure object to a source protocol section from which the clinical procedure object was derived. The system of any embodiment, wherein the structured protocol data object eliminates redundancy inherent in domain-based systems where contextual information is repeated across multiple domain records. The system of any embodiment, wherein the at least one processor is further configured to implement a three-layer decomposition comprising a definition layer encoding intervention identity, a logistics layer encoding handling and storage parameters, and a task layer encoding scheduled visit-level activities. The system of any embodiment, wherein a single intervention definition object in the definition layer is referenced by multiple logistics and task objects without duplication of definition data.

A method for organizing clinical trial protocol data, the method comprising:
generating, by at least one processor, a structured protocol data object wherein a clinical procedure object serves as a primary organizing entity, each clinical procedure object simultaneously comprising: schedule linkage to one or more visit definitions with protocol-defined timing windows; vocabulary identifiers from a plurality of independent clinical terminology standards co-resident on the clinical procedure object; references to applicable protocol rule objects encoding safety, monitoring, and compliance logic; a cross-reference index to an originating protocol document section; and a deterministic anchoring point from which a plurality of downstream operational artifacts are generated; propagating, by the at least one processor, a modification of the clinical procedure object to all derived artifacts referencing the clinical procedure object.

The method of any embodiment, wherein the clinical procedure object conforms to one of fourteen defined service types comprising administration, assessment, ECG, vital signs, laboratory, imaging, biospecimen, informed consent, questionnaire, procedure, concomitant medication, safety monitoring, randomization, and eligibility evaluation.

The method of any embodiment, wherein the vocabulary identifiers comprise identifiers from at least two of LOINC, CDISC, NIH CDE, and SNOMED. The method of any embodiment, further comprising generating the plurality of downstream operational artifacts through deterministic template instantiation against the structured protocol data object.

The method of any embodiment, wherein the protocol rule objects comprise concomitant therapy rule arrays encoding medication class restrictions with status classifications.

The method of any embodiment, further comprising decomposing interventional entities into definition sub-objects, logistics sub-objects, and task sub-objects with explicit referential links between layers.

A non-transitory computer-readable storage medium storing processor-executable instructions that, when executed by at least one processor, cause the at least one processor to: generate a structured protocol data object wherein a clinical procedure object serves as a primary organizing entity, each clinical procedure object simultaneously comprising schedule linkage, vocabulary identifiers from a plurality of independent clinical terminology standards, references to applicable protocol rule objects, a cross-reference index, and a deterministic anchoring point for downstream artifact generation; and propagate a modification of the clinical procedure object to all derived artifacts referencing the clinical procedure object.

The medium of any embodiment, wherein the clinical procedure object conforms to one of a plurality of defined service types, each service type having a type-specific field schema extending a common base schema.

The medium of any embodiment, wherein the vocabulary identifiers comprise a LOINC code, a CDISC domain tag, an NIH CDE reference, and a SNOMED procedure code co-resident on the clinical procedure object.

According to one embodiment, a system for organizing clinical trial protocol data comprises at least one processor operatively connected to a memory. The processor generates a structured protocol data object wherein a clinical procedure object serves as a primary organizing entity. Each clinical procedure object simultaneously comprises schedule linkage to one or more visit definitions with protocol-defined timing windows, vocabulary identifiers from a plurality of independent clinical terminology standards co-resident on the clinical procedure object, references to applicable protocol rule objects encoding safety, monitoring, and compliance logic, a cross-reference index to an originating protocol document section, and a deterministic anchoring point from which a plurality of downstream operational artifacts are generated. Modification of the clinical procedure object propagates to all derived artifacts referencing the clinical procedure object. The clinical procedure object conforms to one of fourteen defined service types comprising administration, assessment, ECG, vital signs, laboratory, imaging, biospecimen, informed consent, questionnaire, procedure, concomitant medication, safety monitoring, randomization, and eligibility evaluation. The vocabulary identifiers comprise identifiers from LOINC, CDISC, NIH CDE, and SNOMED co-resident on the same object.

A system for generating clinical trial operational documents from an unstructured protocol, the system comprising: at least one processor operatively connected to a memory, the at least one processor when executing configured to: instantiate an AI-assisted extraction module configured to receive an unstructured clinical trial protocol document and produce a structured protocol data object by applying natural language processing and entity extraction, wherein all artificial intelligence inference in the system occurs within the AI-assisted extraction module;
present the structured protocol data object to a human validation interface for review and, upon approval, transition the structured protocol data object to a locked state; and instantiate a deterministic generation module configured to produce a plurality of clinical and regulatory operational documents by template instantiation and rule application against the locked structured protocol data object, wherein the deterministic generation module contains no artificial intelligence inference components, no neural network processing components, and no probabilistic computation components.

The system of any embodiment, wherein the AI-assisted extraction module comprises a large language model configured to extract structured data fields from the unstructured clinical trial protocol document. The system of any embodiment, wherein the AI-assisted extraction module is configured to extract conditional logic from the unstructured clinical trial protocol document including dose thresholds, treatment stopping criteria, and branching conditions. The system of any embodiment, wherein the locked state is irreversible such that the locked structured protocol data object cannot be modified without creating a new version requiring validation. The system of any embodiment, wherein the plurality of clinical and regulatory operational documents comprises at least one of EDC configurations, CRF form builds, informed consent documents, treatment checklists, patient visit calendars, grids, and regulatory submission templates. The system of any embodiment, wherein the deterministic generation module generates eligibility criteria mapped to EMR/EHR structured query fields. The system of any embodiment, wherein eligibility evaluation service objects are mapped via deterministic lookup tables to OMOP CDM and HL7 FHIR patient record fields. The system of any embodiment, wherein the deterministic lookup tables generate structured database queries executable against an electronic medical record to identify potentially eligible patients without artificial intelligence inference at a point of query execution.

The system of any embodiment, wherein the locked structured protocol data object serves as an architectural boundary between an AI-permitted zone and an AI-prohibited zone. The system of any embodiment, wherein every output of the deterministic generation module is reproducibly derivable from the locked structured protocol data object.

The system of any embodiment, further comprising a confidence-gated pipeline for assigning clinical vocabulary identifiers, wherein an AI normalization step outputs a canonical name and a confidence signal, a deterministic database lookup returns a candidate code and match confidence score, and a confidence gate writes the vocabulary identifier only when the confidence score meets a predefined threshold.

A method for generating clinical trial operational documents from an unstructured protocol, the method comprising: receiving, by at least one processor, an unstructured clinical trial protocol document; extracting, by an AI-assisted extraction module, structured data from the unstructured clinical trial protocol document to produce a structured protocol data object, wherein all artificial intelligence inference occurs within the AI-assisted extraction module;
presenting the structured protocol data object to a human validator for review; transitioning, upon approval by the human validator, the structured protocol data object to a locked state; and
generating, by a deterministic generation module, a plurality of clinical and regulatory operational documents by template instantiation against the locked structured protocol data object, wherein the deterministic generation module contains no artificial intelligence inference components.

The method of any embodiment, wherein extracting structured data comprises extracting conditional logic including dose adjustment rules, treatment stopping criteria, and branching conditions. The method of any embodiment, further comprising mapping eligibility evaluation service objects to OMOP CDM and HL7 FHIR patient record fields via deterministic lookup without artificial intelligence inference. The method of any embodiment, wherein the plurality of clinical and regulatory operational documents comprises EDC configurations with field names, validation rules, edit checks, and skip logic. The method of any embodiment, further comprising assigning clinical vocabulary identifiers through a confidence-gated pipeline wherein a large language model is architecturally prohibited from generating vocabulary codes directly. The method of any embodiment, wherein the large language model produces a normalized canonical name that feeds a deterministic database query against a vocabulary database.

A non-transitory computer-readable storage medium storing processor-executable instructions that, when executed by at least one processor, cause the at least one processor to:
receive an unstructured clinical trial protocol document; extract, via an AI-assisted extraction module, structured data from the unstructured clinical trial protocol document to produce a structured protocol data object; present the structured protocol data object for human validation and, upon approval, transition the structured protocol data object to a locked state; and
generate, via a deterministic generation module containing no artificial intelligence inference components, a plurality of clinical and regulatory operational documents by template instantiation against the locked structured protocol data object.

The medium of any embodiment, wherein the processor-executable instructions further cause the at least one processor to map eligibility evaluation service objects to OMOP CDM and HL7 FHIR patient record fields via deterministic lookup tables to generate automated pre-screening queries. The medium of any embodiment, wherein the locked structured protocol data object serves as an architectural boundary such that all artificial intelligence inference occurs prior to the locked state and no artificial intelligence inference occurs after the locked state.

According to one embodiment, a system for generating clinical trial operational documents from an unstructured protocol comprises at least one processor operatively connected to a memory. The processor instantiates an AI-assisted extraction module configured to receive an unstructured clinical trial protocol document and produce a structured protocol data object by applying natural language processing and entity extraction, wherein all artificial intelligence inference in the system occurs within the AI-assisted extraction module. The processor presents the structured protocol data object to a human validation interface for review and, upon approval, transitions the structured protocol data object to a locked state. The processor instantiates a deterministic generation module configured to produce a plurality of clinical and regulatory operational documents by template instantiation and rule application against the locked structured protocol data object, wherein the deterministic generation module contains no artificial intelligence inference components, no neural network processing components, and no probabilistic computation components. The locked structured protocol data object serves as an architectural boundary between an AI-permitted zone and an AI-prohibited zone, preventing AI hallucinations from propagating to clinical and regulatory outputs.

A system for automated clinical trial site feasibility assessment, the system comprising: at least one processor operatively connected to a memory, the at least one processor when executing configured to: receive a plurality of validated structured protocol data objects, each representing a clinical trial protocol digitized at procedure-level granularity, each associated with a specific clinical trial site; aggregate procedure-level data from the plurality of validated structured protocol data objects associated with each site to derive a site capability profile comprising procedure type coverage, therapeutic area coverage, protocol complexity indicators derived from structured rule objects within the protocol data, and operational throughput metrics;
receive a new structured protocol data object representing a clinical trial for which site selection is desired; compare requirements of the new structured protocol data object against each candidate site's derived capability profile at a procedure-node level; and produce a ranked feasibility score for each candidate site, wherein the feasibility comparison operates on structured data elements through deterministic field comparison without artificial intelligence inference.

The system of any embodiment, wherein the procedure type coverage comprises coverage across a plurality of defined service types including administration, assessment, ECG, vital signs, laboratory, imaging, biospecimen, informed consent, questionnaire, procedure, concomitant medication, safety monitoring, randomization, and eligibility evaluation. The system of any embodiment, wherein the protocol complexity indicators comprise protocol rules (e.g., protocolRules) logic tree density measuring a number of nodes within Boolean logic tree data structures encoding safety rules. The system of any embodiment, wherein the protocol complexity indicators comprise PK timepoint counts measuring a number of pharmacokinetic sampling timepoints. The system of any embodiment, wherein the protocol complexity indicators comprise biospecimen type diversity measuring a number of distinct biospecimen types collected.

The system of any embodiment, wherein the at least one processor is further configured to generate a capability gap report identifying procedures the new structured protocol data object requires that the site has no demonstrated history with. The system of any embodiment, wherein the site capability profile is derived from validated protocol data objects representing actual conducted studies without reliance on self-reported site questionnaires. The system of any embodiment, wherein the at least one processor is further configured to generate a site comparison ranking across a plurality of candidate sites ordered by feasibility score. The system of any embodiment, wherein the therapeutic area coverage comprises therapeutic area counts accumulated across all studies associated with each site.

The system of any embodiment, wherein the site capability profile further comprises study phase coverage indicating which trial phases the site has demonstrated experience conducting. The system of any embodiment, wherein the operational throughput metrics comprise at least one of enrollment rates, protocol deviation rates, and study completion rates computed from patient-level protocol data records.

A method for automated clinical trial site feasibility assessment, the method comprising: receiving, by at least one processor, a plurality of validated structured protocol data objects, each representing a clinical trial protocol digitized at procedure-level granularity, each associated with a specific clinical trial site; aggregating, by the at least one processor, procedure-level data from the plurality of validated structured protocol data objects associated with each site to derive a site capability profile comprising procedure type coverage, therapeutic area coverage, and protocol complexity indicators; receiving a new structured protocol data object representing a clinical trial for which site selection is desired; comparing requirements of the new structured protocol data object against each candidate site's derived capability profile at a procedure-node level through deterministic field comparison; and producing a ranked feasibility score for each candidate site without artificial intelligence inference at a matching step. The method of any embodiment, wherein the protocol complexity indicators comprise protocol rules (e.g., protocolRules) logic tree density, PK timepoint counts, and biospecimen type diversity. The method of any embodiment, further comprising generating a capability gap report identifying procedures required by the new structured protocol data object for which the site capability profile contains no demonstrated experience. The method of any embodiment, wherein the site capability profile is derived from validated protocol data objects without reliance on external databases or self-reported questionnaires. The method of any embodiment, further comprising generating a site comparison ranking ordering candidate sites by feasibility score to enable automated site selection support. The method of any embodiment, wherein aggregating procedure-level data comprises extracting service categories providing finer granularity than procedure types by distinguishing between different clinical activities within each procedure type.

A non-transitory computer-readable storage medium storing processor-executable instructions that, when executed by at least one processor, cause the at least one processor to: receive a plurality of validated structured protocol data objects associated with clinical trial sites; aggregate procedure-level data from the plurality of validated structured protocol data objects to derive a site capability profile for each site comprising procedure type coverage, therapeutic area coverage, and protocol complexity indicators; receive a new structured protocol data object representing a clinical trial for which site selection is desired; compare requirements of the new structured protocol data object against each site's capability profile through deterministic field comparison; and produce a ranked feasibility score for each candidate site without artificial intelligence inference.

The medium of any embodiment, wherein the protocol complexity indicators comprise protocol rules (e.g., protocolRules) logic tree density, PK timepoint counts, and biospecimen type diversity derived from structured rule objects within the validated structured protocol data objects. The medium of any embodiment, wherein the processor-executable instructions further cause the at least one processor to generate a capability gap report and a site comparison ranking based on the feasibility comparison. According to one embodiment, a system for automated clinical trial site feasibility assessment comprises at least one processor operatively connected to a memory. The processor receives a plurality of validated structured protocol data objects, each representing a clinical trial protocol digitized at procedure-level granularity, each associated with a specific clinical trial site. The processor aggregates procedure-level data from the plurality of validated structured protocol data objects associated with each site to derive a site capability profile comprising procedure type coverage, therapeutic area coverage, protocol complexity indicators derived from structured rule objects within the protocol data, and operational throughput metrics. The protocol complexity indicators comprise protocol rules (e.g., protocolRules) logic tree density, PK timepoint counts, and biospecimen type diversity. The processor receives a new structured protocol data object representing a clinical trial for which site selection is desired, compares requirements of the new structured protocol data object against each candidate site's derived capability profile at a procedure-node level, and produces a ranked feasibility score for each candidate site through deterministic field comparison without artificial intelligence inference.

According to one embodiment, a system for data management is provided. The system comprises: at least one processor operatively connected to a memory, the at least one processor when executing configured to: accept input of unstructured or multi-format source data (e.g., clinical trial data); process the unstructured or multi-format data into a compressed structured data format, wherein the compressed data format is organized on procedure-based associations linking procedure-based information to associated source data (e.g., clinical trial data); wherein the operations executed to process the unstructured or multi-format data include: instantiate a machine learning model configured to analyze protocol data (e.g., clinical protocol data) to identify procedure-based information and associated source data (e.g., clinical trial data) in response to input of unstructured or multi-format source data (e.g., clinical trial data); and output the compressed data format, including respective procedure, procedure-based associations, and associated source data (e.g., clinical trial data).

According to any embodiment, the operations executed to process the unstructured or multi-format data include execution of a plurality of rules to identify procedure-based information in unstructured or a plurality of non-homogeneous data sources. According to any embodiment, the execution of the plurality of rules includes mapping identified procedures to associated source data (e.g., clinical trial data). According to any embodiment, the compressed structured data format comprises a procedure-based paradigm in which a procedure object (e.g., clinical procedure object) serves as a primary organizing entity, and includes at least a node storing executable code for downstream systems. According to any embodiment, each procedure object (e.g., clinical procedure object) simultaneously comprises schedule linkage to one or more visit definitions with protocol-defined timing windows, vocabulary identifiers from a plurality of independent terminology standards (e.g., clinical terminology standards), references to applicable protocol rule objects, and a cross-reference index to an originating protocol document section. According to any embodiment, the vocabulary identifiers comprise identifiers from at least two of the plurality of independent terminology standards (e.g., LOINC, CDISC, NIH CDE, and SNOMED) co-resident on the procedure object (e.g., clinical procedure object).

According to any embodiment, wherein the procedure object (e.g., clinical procedure object) conforms to one of a plurality of defined service types comprising administration, assessment, ECG, vital signs, laboratory, imaging, biospecimen, informed consent, questionnaire, procedure, concomitant medication, safety monitoring, randomization, and eligibility evaluation. According to any embodiment, wherein the at least one processor is further configured to automatically generate a plurality of downstream operational artifacts from the compressed structured data format. According to any embodiment, wherein the plurality of downstream operational artifacts comprises at least one of execution checklists (e.g., treatment checklists), grid data (e.g., billing grids), calendars (e.g., patient calendars), template drafts (e.g., informed consent form), templates (e.g., EDC templates), or dispensation schedules (e.g., pharmacy dispensation schedules). According to any embodiment, wherein modification of a procedure object in the compressed structured data format propagates to all derived artifacts referencing the procedure object.

According to any embodiment, the machine learning model comprises a large language model configured to extract structured data fields from the unstructured or multi-format source data (e.g., clinical trial data) and to extract conditional logic, including dose thresholds, treatment stopping criteria, and branching conditions. According to one embodiment, a method for data management is provided. The method comprises: accepting, by at least one processor, input of unstructured or multi-format source data (e.g., clinical trial data); processing the unstructured or multi-format data into a compressed structured data format, wherein the compressed data format is organized on procedure-based associations linking procedure-based information to associated source data (e.g., clinical trial data); instantiating a machine learning model configured to analyze protocol data (e.g., clinical protocol data) to identify procedure-based information and associated source data (e.g., clinical trial data); and outputting the compressed data format including respective procedure, procedure-based associations, and associated source data (e.g., clinical trial data). According to any embodiment, the method further comprises executing a plurality of rules to identify procedure-based information in unstructured or a plurality of non-homogeneous data sources. According to any embodiment, executing the plurality of rules includes mapping identified procedures to associated source data (e.g., clinical trial data).

According to any embodiment, the method further comprising automatically generating a plurality of downstream operational artifacts from the compressed structured data format, wherein the plurality of downstream operational artifacts comprises at least one of execution checklists (e.g., treatment), grid data, calendars (e.g., patient), template drafts (e.g., informed consent form), templates (e.g., EDC templates), or dispensation schedules (e.g., pharmacy dispensation schedules). According to any embodiment, the method further comprises distributing the compressed structured data format to a plurality of clinical trial sites via an application programming interface. According to any embodiment, the compressed structured data format implements a cross-reference architecture in which procedures link to associated data elements through reference identifiers rather than embedded copies, and includes at least a node storing executable code for downstream systems.

According to one embodiment, a non-transitory computer-readable storage medium storing processor-executable instructions is provided. The instructions when executed by at least one processor, cause the at least one processor to: accept input of unstructured or multi-format source data (e.g., clinical trial data); process the unstructured or multi-format data into a compressed structured data format organized on procedure-based associations linking procedure-based information to associated source data (e.g., clinical trial data); instantiate a machine learning model configured to analyze protocol data (e.g., clinical protocol data) to identify procedure-based information and associated source data (e.g., clinical trial data); and output the compressed data format including respective procedure, procedure-based associations, and associated source data (e.g., clinical trial data).

According to any embodiment, the processor-executable instructions further cause the at least one processor to execute a plurality of rules to identify procedure-based information and map identified procedures to associated source data (e.g., clinical trial data). According to any embodiment, the processor-executable instructions further cause the at least one processor to automatically generate a plurality of downstream operational artifacts comprises at least one of execution checklists (e.g., treatment), grid data, calendars (e.g., patient), template drafts (e.g., informed consent form), templates (e.g., EDC templates), or dispensation schedules (e.g., pharmacy dispensation schedules).

A system for database management of protocol data comprises at least one processor operatively connected to a memory, the at least one processor when executing configured to generate a structured data object wherein a procedure object (e.g., clinical procedure object) operates as a primary organizing data entity, the structured data object comprising schedule linkage to one or more visit definitions with defined timing windows, vocabulary identifiers from a plurality of independent terminology standards (e.g., independent clinical terminology standards) co-resident on the procedure object, the plurality of independent terminology standards including at least two standards mapped to internal data, references to applicable protocol rule objects encoding safety, monitoring, and compliance logic, a cross-reference index to an originating source section, and a deterministic anchoring point in the structured data object from which a plurality of derived downstream executable elements are linked, wherein modification of the procedure object propagates to all derived elements referencing the procedure object.

The system of any embodiment, wherein the at least one processor is configured to receive protocol data (e.g., clinical trial protocol data) containing safety rules expressed in natural language, encode a protocol rules section into the structured data object, wherein the protocol rules section includes adverse event logic tree objects encoding Boolean evaluation criteria with at least one of nested operator nodes, parameter references (e.g., clinical parameter references), comparators, threshold values, or child node arrays, safety threshold objects encoding graded severity monitoring criteria with at least one of parameter identifiers (e.g., clinical parameter identifiers) conforming to an external vocabulary standard, action triggers, or severity grades, and concomitant rule arrays (e.g., therapy rule arrays) encoding class restrictions (e.g., medication class restrictions) with status classifications including at least one of allowed, prohibited, or restricted designations, washout period durations, or conditional exception logic, and store the structured protocol data object in a machine-readable format, enabling computational evaluation of the safety rules against patient clinical data, wherein the protocol rules section further includes discontinuation criteria objects encoding evaluable condition trees with criterion type classifications, required action arrays, and follow-up plan references or the criterion type classifications include at least one of decision status (e.g., investigator_decision), protocol designation (e.g., protocol_specified), sponsor metadata (e.g., sponsor_directed), or patient metadata (e.g., patient_withdrawal) designations, or wherein the protocol rules section further includes follow-up rule objects encoding post-event clinical scheduling obligations with trigger event references, visit window arrays, required service arrays, and termination condition trees or wherein the trigger event references include SAE or status (e.g., treatment_discontinuation, study_completion, etc.) designations.

The system of any embodiment, wherein the procedure object conforms to one of a plurality of defined service types comprising at least one of administration, assessment, ECG, vital signs, laboratory, imaging, biospecimen, informed consent, questionnaire, procedure, concomitant medication, safety monitoring, randomization, or eligibility evaluation, wherein each service type has a type-specific field schema extending a common base schema, wherein the vocabulary identifiers comprise at least one of a code (e.g., LOINC) identifying a measurement (e.g., clinical measurement), a domain tag for mapping (e.g., CDISC domain tag for SDTM mapping), a reference (e.g., NIH CDE reference) for cross-study harmonization, and a code designation (e.g., SNOMED procedure code), wherein the protocol rule objects comprise adverse event logic tree objects encoding Boolean evaluation criteria, or wherein the protocol rule objects comprise safety threshold objects encoding graded severity monitoring criteria with action triggers.

The system of any embodiment, wherein the derived downstream executable elements comprise at least one of EDC configurations, CRF form builds, informed consent documents, treatment checklists, patient visit calendars, grids, and regulatory submission templates, wherein the cross-reference index enables traceability from the procedure object to a source section from which the procedure object was derived, wherein the structured data object eliminates redundancy inherent in domain-based systems where contextual information is repeated across multiple domain records, wherein the at least one processor is further configured to implement a three-layer decomposition comprising a definition layer encoding intervention identity, a logistics layer encoding handling and storage parameters, and a task layer encoding scheduled visit-level activities when encoding the structured data object, or wherein a single intervention definition object in the definition layer is referenced by multiple logistics and task objects without duplication of definition data.

A method for organizing protocol data comprises generating, by at least one processor, a structured data object wherein a procedure object (e.g., clinical procedure object) operates as a primary organizing data entity, the structured data object comprising schedule linkage to one or more visit definitions with defined timing windows, vocabulary identifiers from a plurality of independent terminology standards (e.g., independent clinical terminology standards) co-resident on the procedure object, the plurality of independent clinical terminology standards including at least two standards mapped to internal data, references to applicable protocol rule objects encoding safety, monitoring, and compliance logic, a cross-reference index to an originating source section, and a deterministic anchoring point in the structured data object from which a plurality of derived downstream executable elements are linked, and propagating, by the at least one processor, a modification of the procedure object to all derived elements referencing the procedure object.

The method of any embodiment, wherein the procedure object conforms to one of fourteen defined service types comprising administration, assessment, ECG, vital signs, laboratory, imaging, biospecimen, informed consent, questionnaire, procedure, concomitant medication, safety monitoring, randomization, and eligibility evaluation, wherein the vocabulary identifiers comprise at least one of a code (e.g., LOINC) identifying a measurement (e.g., clinical measurement), a domain tag for mapping (e.g., CDISC domain tag for SDTM mapping), a reference (e.g., NIH CDE reference) for cross-study harmonization, and a code designation (e.g., SNOMED procedure code), further comprising generating the plurality of derived downstream executable elements through deterministic template instantiation against the structured data object, wherein the protocol rule objects comprise concomitant rule arrays (e.g., therapy rule arrays) encoding class restrictions (e.g., medication class restrictions) with status classifications, or further comprising decomposing interventional entities into definition sub-objects, logistics sub-objects, and task sub-objects with explicit referential links between layers.

A non-transitory computer-readable storage medium stores processor-executable instructions that, when executed by at least one processor, cause the at least one processor to generate a structured data object wherein a procedure object (e.g., clinical procedure object) operates as a primary organizing data entity, the structured data object comprising schedule linkage to one or more visit definitions with defined timing windows, vocabulary identifiers from a plurality of independent terminology standards (e.g., independent clinical terminology standards) co-resident on the procedure object, the plurality of independent clinical terminology standards including at least two standards mapped to internal data, references to applicable protocol rule objects encoding safety, monitoring, and compliance logic, a cross-reference index to an originating source section, and a deterministic anchoring point in the structured data object from which a plurality of derived downstream executable elements are linked, and propagate a modification of the procedure object to all derived elements referencing the procedure object.

The medium of any embodiment, or wherein the procedure object conforms to one of a plurality of defined service types, each service type having a type-specific field schema extending a common base schema, wherein vocabulary identifiers comprise at least one of a code (e.g., LOINC) identifying a measurement (e.g., clinical measurement), a domain tag for mapping (e.g., CDISC domain tag for SDTM mapping), a reference (e.g., NIH CDE reference) for cross-study harmonization, and a code designation (e.g., SNOMED procedure code).

The terms “program” or “software” or “app” are used herein in a generic sense to refer to any type of computer code or set of processor-executable instructions that can be employed to program a computer or other processor to implement various aspects of embodiments as discussed above. Additionally, it should be appreciated that according to one aspect, one or more computer programs that when executed perform methods of the disclosure provided herein need not reside on a single computer or processor, but may be distributed in a modular fashion among different computers or processors to implement various aspects of the disclosure provided herein. Processor-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in one or more non-transitory computer-readable storage media in any suitable form. For simplicity of illustration, data structures may be shown to have fields that are related through location in the data structure. Such relationships may likewise be achieved by assigning storage for the fields with locations in a non-transitory computer-readable medium that convey relationships between the fields. However, any suitable mechanism may be used to establish relationships among information in fields of a data structure, including through the use of pointers, tags or other mechanisms that establish relationships among data elements.

Also, various inventive concepts may be embodied as one or more processes, of which examples have been provided. The acts performed as part of each process may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, and/or ordinary meanings of the defined terms. As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements.

This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having the same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items. Having described several embodiments of the techniques described herein in detail, various modifications, and improvements will readily occur to those skilled in the art. Such modifications and improvements are intended to be within the spirit and scope of the disclosure. Accordingly, the foregoing description is by way of example only, and is not intended as limiting. The techniques are limited only as defined by the following claims and the equivalents thereto.

1. A system for database management of protocol data, the system comprising:
at least one processor operatively connected to a memory, the at least one processor when executing configured to:
generate a structured data object wherein a procedure object operates as a primary organizing data entity, the structured data object comprising:
schedule linkage to one or more visit definitions with defined timing windows;
vocabulary identifiers from a plurality of independent terminology standards co-resident on the procedure object, the plurality of independent terminology standards including at least two standards mapped to internal data;
references to applicable protocol rule objects encoding safety, monitoring, and compliance logic;
a cross-reference index to an originating source section; and
a deterministic anchoring point in the structured data object from which a plurality of derived downstream executable elements are linked; and
wherein modification of the procedure object propagates to all derived elements referencing the procedure object.

2. The system of claim 1, wherein the at least one processor is configured to:
receive protocol data containing safety rules expressed in natural language; encode a protocol rules section into the structured data object, wherein the protocol rules section includes:
adverse event logic tree objects encoding Boolean evaluation criteria with at least one of nested operator nodes, parameter references, comparators, threshold values, or child node arrays; safety threshold objects encoding graded severity monitoring criteria with at least one of parameter identifiers conforming to an external vocabulary standard, action triggers, or severity grades; and
concomitant rule arrays encoding class restrictions with status classifications including at least one of allowed, prohibited, or restricted designations, washout period durations, or conditional exception logic; and
store the structured protocol data object in a machine-readable format, enabling computational evaluation of the safety rules against patient clinical data.

3. The system of claim 2, wherein the protocol rules section further includes discontinuation criteria objects encoding evaluable condition trees with criterion type classifications, required action arrays, and follow-up plan references or the criterion type classifications include at least one of decision status, protocol designation, sponsor metadata, or patient metadata designations.

4. The system of claim 2, wherein the protocol rules section further includes follow-up rule objects encoding post-event clinical scheduling obligations with trigger event references, visit window arrays, required service arrays, and termination condition trees or wherein the trigger event references include SAE or status designations.

5. The system of claim 1, wherein the procedure object conforms to one of a plurality of defined service types comprising at least one of administration, assessment, ECG, vital signs, laboratory, imaging, biospecimen, informed consent, questionnaire, procedure, concomitant medication, safety monitoring, randomization, or eligibility evaluation.

6. The system of claim 5, wherein each service type has a type-specific field schema extending a common base schema.

7. The system of claim 1, wherein the vocabulary identifiers comprise at least one of a code identifying a measurement, a domain tag for mapping, a reference for cross-study harmonization, and a code designation.

8. The system of claim 1, wherein the protocol rule objects comprise adverse event logic tree objects encoding Boolean evaluation criteria.

9. The system of claim 1, wherein the protocol rule objects comprise safety threshold objects encoding graded severity monitoring criteria with action triggers.

10. The system of claim 1, wherein the derived downstream executable elements comprise at least one of EDC configurations, CRF form builds, informed consent documents, treatment checklists, patient visit calendars, grids, and regulatory submission templates.

11. The system of claim 1, wherein the cross-reference index enables traceability from the procedure object to a source section from which the procedure object was derived.

12. The system of claim 1, wherein the structured data object eliminates redundancy inherent in domain-based systems where contextual information is repeated across multiple domain records.

13. The system of claim 1, wherein the at least one processor is further configured to implement a three-layer decomposition comprising a definition layer encoding intervention identity, a logistics layer encoding handling and storage parameters, and a task layer encoding scheduled visit-level activities when encoding the structured data object.

14. The system of claim 13, wherein a single intervention definition object in the definition layer is referenced by multiple logistics and task objects without duplication of definition data.

15. A method for organizing protocol data, the method comprising:
generating, by at least one processor, a structured data object wherein a procedure object operates as a primary organizing data entity, the structured data object comprising:
schedule linkage to one or more visit definitions with defined timing windows;
vocabulary identifiers from a plurality of independent terminology standards co-resident on the procedure object, the plurality of independent terminology standards including at least two standards mapped to internal data;
references to applicable protocol rule objects encoding safety, monitoring, and compliance logic;
a cross-reference index to an originating source section; and
a deterministic anchoring point in the structured data object from which a plurality of derived downstream executable elements are linked; and
propagating, by the at least one processor, a modification of the procedure object to all derived elements referencing the procedure object.

16. The method of claim 15, wherein the procedure object conforms to one of fourteen defined service types comprising administration, assessment, ECG, vital signs, laboratory, imaging, biospecimen, informed consent, questionnaire, procedure, concomitant medication, safety monitoring, randomization, and eligibility evaluation.

17. The method of claim 15, wherein the vocabulary identifiers comprise at least one of a code identifying a measurement, a domain tag for mapping, a reference for cross-study harmonization, and a code designation.

18. The method of claim 15, further comprising generating the plurality of derived downstream executable elements through deterministic template instantiation against the structured data object.

19. The method of claim 15, wherein the protocol rule objects comprise concomitant rule arrays encoding class restrictions with status classifications.

20. The method of claim 15, further comprising decomposing interventional entities into definition sub-objects, logistics sub-objects, and task sub-objects with explicit referential links between layers.
